(19) United States
(12) Patent Application Publication
(10) Pub. No.: US 2014/0206888 A1
(43) Pub. Date: Jul. 24, 2014

(54) METHODS FOR PREPARATION OF THIOPHENE COMPOUNDS

(71) Applicant: Vertex Pharmaceuticals Incorporated, Boston, MA (US)

(72) Inventors: Valdas Jurkauskas, Cambridge, MA (US); Piero L. Ruggiero, Cambridge, MA (US); Stefanie Roeper, Medford, MA (US); David Willcox, Wellesley, MA (US); Michael Waldo, Grafton, MA (US); Dahrika Milfred Yap Guerette, Cambridge, MA (US); Billie J. Kline, Kingston, MA (US); Hoa Q. Luong, Newton, MA (US)

(73) Assignee: Vertex Pharmaceuticals Incorporated, Boston, MA (US)

(21) Appl. No.: 14/163,036

(22) Filed: Jan. 24, 2014

Related U.S. Application Data
(63) Continuation of application No. PCT/US2012/048270, filed on Jul. 26, 2012.
(60) Provisional application No. 61/511,648, filed on Jul. 26, 2011.
(60) Provisional application No. 61/511,647, filed on Jul. 26, 2011.
(60) Provisional application No. 61/545,751, filed on Oct. 11, 2011.
(60) Provisional application No. 61/512,079, filed on Jul. 27, 2011.
(60) Provisional application No. 61/511,644, filed on Jul. 26, 2011.
(60) Provisional application No. 61/623,144, filed on Apr. 12, 2012.

(51) Int. Cl. C07D 409/12; C07D 333/40

(52) U.S. Cl. CPC C07D 409/12; C07D 333/40
USPC 549/60; 549/69

(57) ABSTRACT

A method of preparing Compound (1):

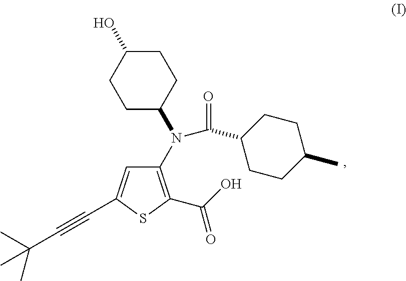

or a pharmaceutically acceptable salt thereof includes: a) reacting Compound (A) with 3,3-dimethylbut-1-yne in the presence of one or more palladium catalysts selected from the group consisting of Pd(PPh₃)₄ and Pd(PPh₃)₂Cl₂, and one or more copper catalysts selected from the group consisting of CuI, CuBr, and CuCl, to generate Compound (B); b) treating Compound (B) with an acid to generate Compound (C): c) reducing the cyclohexanone of Compound (C) to cyclohexanol to generate Compound (D); and d) reacting Compound (D) with a base to generate Compound (1), wherein Compounds (A), (B), (C), and (D) are each as depicted herein.

### RELATED APPLICATIONS

This application is a continuation of PCT Application No. PCT/US2012/048270, filed Jul. 26, 2012, which in turn claims priority to U.S. Provisional Application No. 61/511,648, filed Jul. 26, 2011; U.S. Provisional Application No. 61/511,647, filed Jul. 26, 2011; U.S. Provisional Application No. 61/512,079, filed Jul. 27, 2011; U.S. Provisional Application No. 61/511,644, filed Jul. 26, 2011; U.S. Provisional Application No. 61/545,751, filed Oct. 11, 2011; and U.S. Provisional Application No. 61/623,144, filed Apr. 12, 2012. The entire teachings of these applications are incorporated herein by reference.

### BACKGROUND OF THE INVENTION

Hepatitis C virus (HCV) is a positive-stranded RNA virus belonging to the Flaviviridae family and has closest relationship to the pestiviruses that include hog cholera virus and bovine viral diarrhea virus (BVDV). HCV is believed to replicate through the production of a complementary negative-strand RNA template. Due to the lack of efficient culture replication system for the virus, HCV particles were isolated from pooled human plasma and shown, by electron microscopy, to have a diameter of about 50-60 nm. The HCV genome is a single-stranded, positive-sense RNA of about 9,600 bp coding for a polyprotein of 3009-3030 amino-acids, which is cleaved co and post-translationally into mature viral proteins (core, E1, E2, p7, NS2, NS3, NS4A, NS4B, NS5A, NS5B). It is believed that the structural glycoproteins, E1 and E2, are embedded into a viral lipid envelope and form stable heterodimers. It is also believed that the structural core protein interacts with the viral RNA genome to form the nucleocapsid. The nonstructural proteins designated NS2 to NS5 include proteins with enzymatic functions involved in virus replication and protein processing including a polymerase, protease and helicase.

The main source of contamination with HCV is blood. The magnitude of the HCV infection as a health problem is illustrated by the prevalence among high-risk groups. For example, 60% to 90% of hemophiliacs and more than 80% of intravenous drug abusers in western countries are chronically infected with HCV. For intravenous drug abusers, the prevalence varies from about 28% to 70% depending on the population studied. The proportion of new HCV infections associated with post-transfusion has been markedly reduced lately due to advances in diagnostic tools used to screen blood donors.

Combination of pegylated interferon plus ribavirin is the treatment of choice for chronic HCV infection. This treatment does not provide sustained viral response (SVR) in a majority of patients infected with the most prevalent genotype (1a and 1b). Furthermore, significant side effects prevent compliance to the current regimen and may require dose reduction or discontinuation in some patients.

Until very recently, the standard of care (SOC) for the treatment of HCV infection comprised 48-week administration of a combination of pegylated interferon-α (subcutaneous weekly injection) and ribavirin (oral, twice daily). Therapy was poorly tolerated and ultimately successful in less than half of the treated patient population. Recently two new treatment regimens for HCV patients have been approved by the FDA that comprise a protease inhibitor (telaprevir or boceprevir) in combination of Peg-IFN/ribavirin. These treatments have demonstrated significantly higher cure rates (sustained viral response (SVR)) in clinical trials in comparison to the then SOC (Peg-IFN/RBV) and are expected to increase treatment success rates (SVR) for HCV patients. There is therefore a great need for the continued development of anti-viral agents for use in treating or preventing Flavivirus infections.

### SUMMARY OF THE INVENTION

The present invention generally relates to a method of preparing anti-viral agents, such as Compound (1) or pharmaceutically acceptable salt thereof.

In one embodiment, the present invention is directed to a method of preparing Compound (1) represented by the following structural formula:

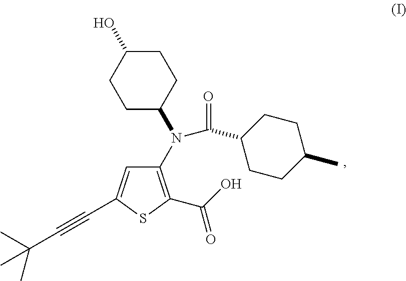

or a pharmaceutically acceptable salt thereof. The method comprises:

a) reacting Compound (A) with 3,3-dimethylbut-1-yne in the presence of one or more palladium catalysts selected from the group consisting of Pd(PPh₃)₄ and Pd(PPh₃)₂Cl₂, and one or more copper catalysts selected from the group consisting of CuI, CuBr, and CuCl, to generate Compound (B);

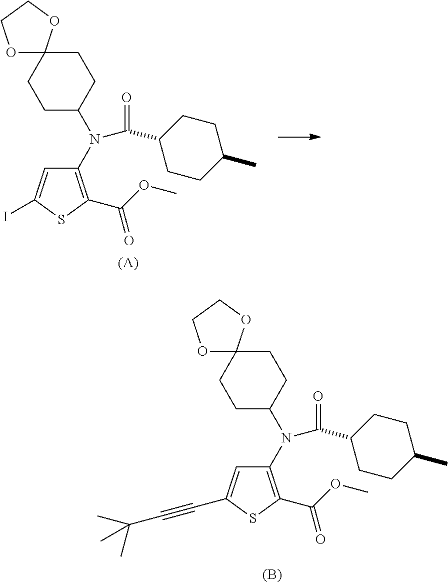

b) treating Compound (B) with an acid to generate Compound (C):

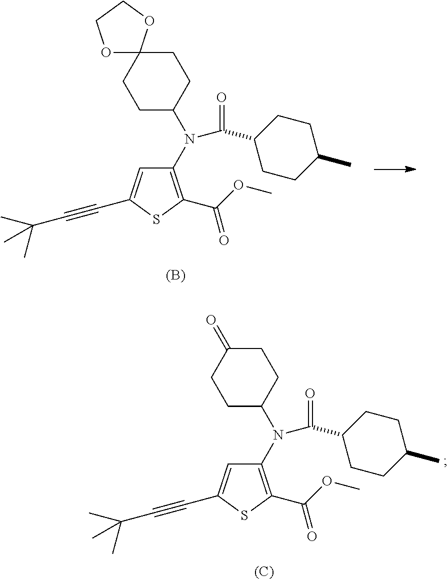

c) reducing the cyclohexanone of Compound (C) to cyclohexanol to generate Compound (D):

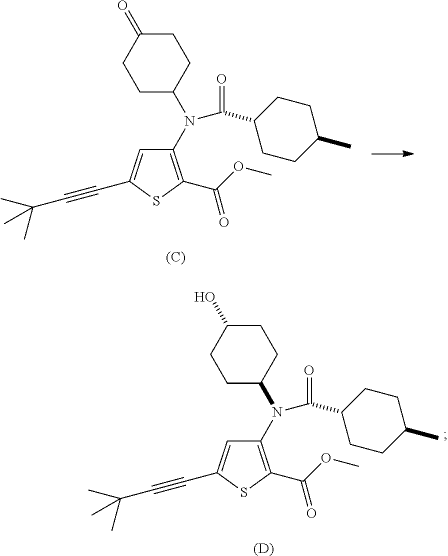

and

d) reacting Compound (D) with a base to generate Compound (1).

In another embodiment, the present invention is directed to a method of preparing Compound (1) or a pharmaceutically acceptable salt thereof. The method comprises:
a) reducing the cyclohexanone of Compound (C) to cyclohexanol in the presence of LiAlH(O^{t}Bu)₃ in an amount of 1.0 to 1.5 equivalents based on molar amount of Compound (C) at a temperature in a range of −70° C. to −35° C. to generate Compound (D):

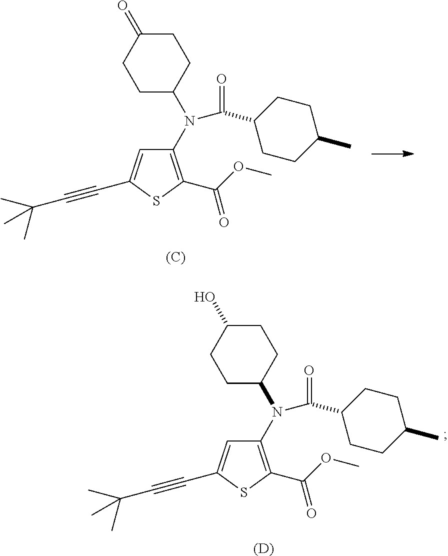

and
b) reacting Compound (D) with a base to generate Compound (1), wherein ^{t}Bu is t-butyl.

In yet another embodiment, the present invention is directed to a method of preparing Compound (1) or a pharmaceutically acceptable salt thereof. The method comprises:
a) reacting Compound (J) with Compound (K) to produce Compound (G) by combining them with NaBH(OAc)₃ and trichloroacetic acid, wherein Ac is acetyl:

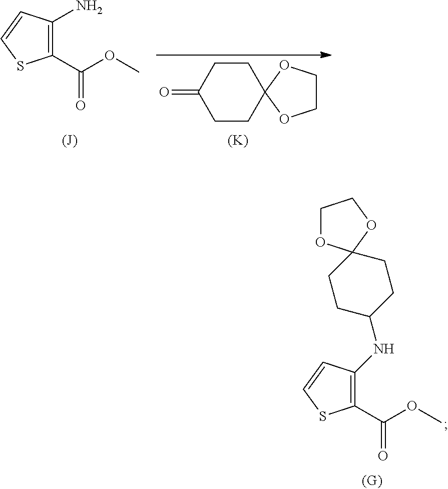

b) amidating Compound (G) with Compound (F) to produce Compound (E):

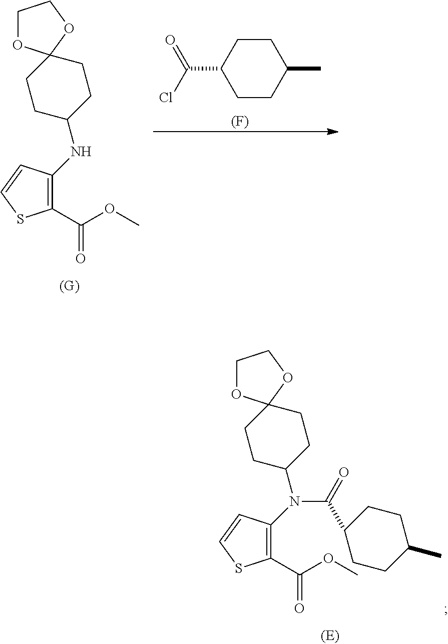

c) reacting Compound (E) with I₂ to produce Compound (A):

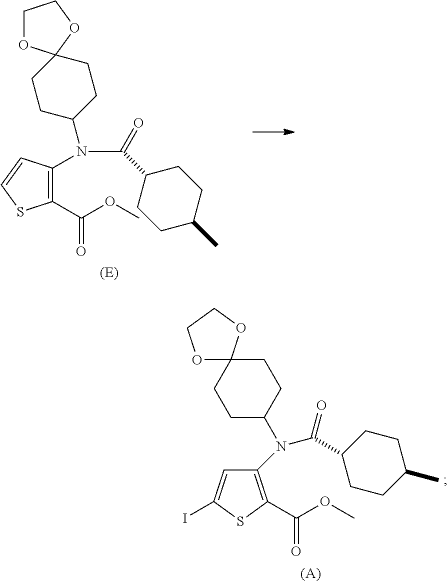

d) reacting Compound (A) with 3,3-dimethylbut-1-yne in the presence of one or more palladium catalysts selected from the group consisting of Pd(PPh₃)₄ and Pd(PPh₃)Cl₂, and one or more copper catalysts selected from the group consisting of CuI, CuBr, and CuCl, wherein the palladium catalyst is in an amount of 0.1 mol % to 0.5 mol % and the copper catalyst is in an amount of 1 mol % to 5 mol %, to generate Compound (B);

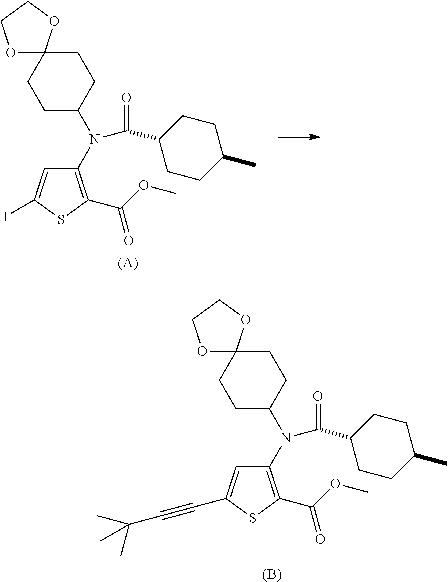

e) treating Compound (B) with an acid to generate Compound (C):

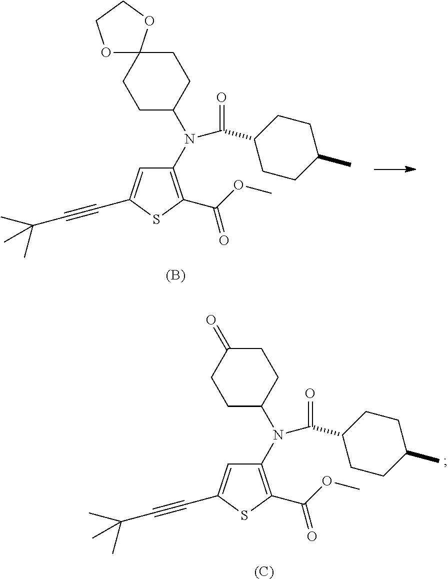

f) reducing the cyclohexanone of Compound (C) to cyclohexanol to generate Compound (D) by the use of LiAlH(O^{t}Bu)₃ in an amount of 1.0 to 1.5 equivalents based on molar amount of Compound (C) at a temperature in a range of −70° C. to −35° C., wherein ^{t}Bu is tert-butyl:

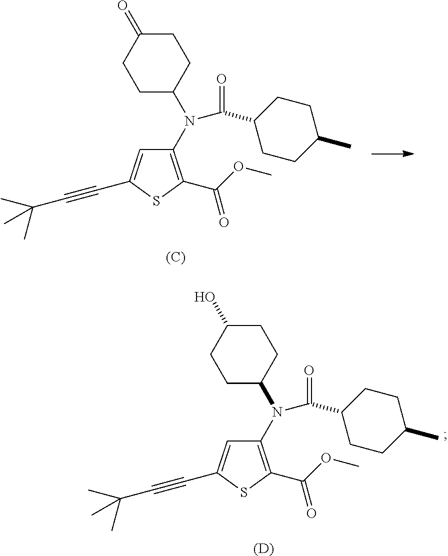

and

reacting Compound (D) with a base to generate Compound (1).

In yet another embodiment, the present invention is directed to a method of preparing Compound (B):

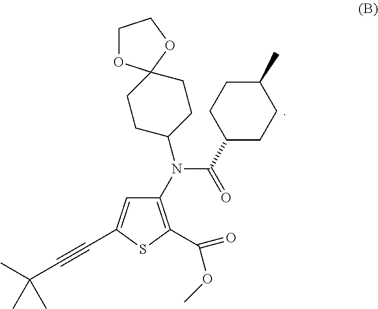

The method comprises reacting Compound (A) with 3,3-dimethylbut-1-yne in the presence of one or more palladium catalysts selected from the group consisting of Pd(PPh₃)₄ and Pd(PPh₃)₂Cl₂, and one or more copper catalysts selected from the group consisting of CuI, CuBr, and CuCl, to generate Compound (B):

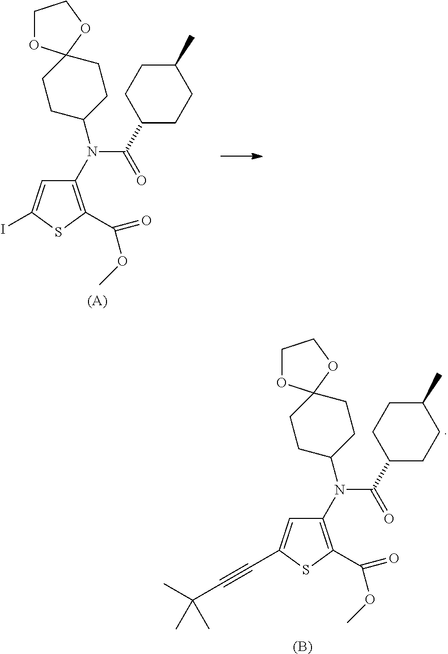

### DETAILED DESCRIPTION OF THE INVENTION

Compound (1) represented by the following structural formula:

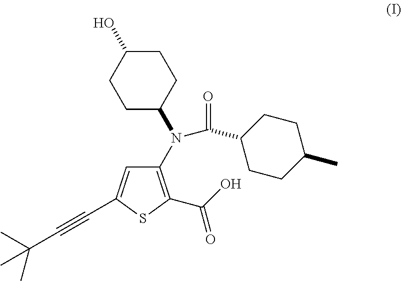

and pharmaceutically acceptable salts thereof are NS5B polymerase inhibitors, and also described in WO 2008/058393.

In one embodiment, Compound (1) can be prepared by employing Step 4 of general scheme 1: reacting Compound (A) with 3,3-dimethylbut-1-yne in the presence of one or more palladium catalysts selected from the group consisting of Pd(PPh₃)₄ and Pd(PPh₃)₂Cl₂, and one or more copper catalysts selected from the group consisting of CuI, CuBr, and CuCl, to generate Compound (B) under suitable conditions, for example in the presence of a base. Any suitable conditions known in the art can be employed for this step. In one specific embodiment, Step 4 is performed in the presence of Et₃N and/or ⁱPr₂NH. Typically, the palladium catalyst is present in an amount 0.1 mol % to 0.5 mol %, such as 0.15 mol % to 0.3 mol % (e.g., 0.2 mol %). Typically, the copper catalyst is present in an amount 1 mol % to 5 mol %, such as 2.5 mol % to 5 mol % or 2.5 mol % to 3.5 mol % (e.g., 3 mol %). Generally, the amount of 3,3-dimethylbut-1-yne is in a range of 1 to 1.5 equivalents to Compound (A), such as 1.1 to 1.3 equivalents to Compound (A).

Any suitable solvent system can be employed for the reaction of Compound (A) with 3,3-dimethylbut-1-yne. Suitable examples include 2-methyl tetrahydrofuran (2-Me THF), dimethylformamide (DMF), methylethyl ketone (MEK or 2-butanone), ethylacetate (EtOAc), methyl t-butyl ether (MtBE), dichloromethane (DCM), toluene, and a mixture thereof. In one specific embodiment, 2-methyl tetrahydrofuran (2-Me THF) or methyl t-butyl ether (MtBE) is employed. In another specific embodiment, the reaction of Compound (A) with 3,3-dimethylbut-1-yne is performed in the presence of Pd(PPh₃)₄ and CuI in 2-methyl tetrahydrofuran (2-Me THF) or methyl t-butyl ether (MtBE).

In another specific embodiment, the reaction of Compound (A) with 3,3-dimethylbut-1-yne is followed by washing the reaction mixture with an aqueous oxalic acid (e.g., 12.6 wt % aqueous oxalic acid and/or 6 wt % aqueous oxalic acid) at least twice. For example, the washing can be done by: i) adding a first washing of aqueous oxalic acid (e.g., 12.6 wt % aqueous oxalic acid) into the reaction mixture Compound (A) with 3,3-dimethylbut-1-yne while maintain the temperature of the mixture below 20° C.-25° C.; ii) stirring the resulting mixture of step i) at a temperature of 20° C.-25° C.; iii) adding a second washing of aqueous oxalic acid (e.g., 6 wt % aqueous oxalic acid) into the resulting mixture of step ii) while maintain the temperature of the mixture below 20° C.-25° C.; and then iv) subsequently stirring the resulting mixture of step iii) at a temperature of 20-25° C. The oxalic acid washing generally generates a biphasic mixture: organic and aqueous layers. Optionally, the desired organic layer is further treated with activated carbon. Without being bound to a particular theory, the aqueous oxalic acid washing and the treatment with activated carbon can reduce the level of residual palladium and copper substantially.

Step 4 employing the palladium and copper catalysts can be performed at a temperature in a range of 18° C. to 30° C. (e.g., 20° C. to 25° C.). Without being bound to a particular theory, performing the reaction at such a low temperature without heating can prevent any potential decomposition of the Pd and/or copper catalysts, and thus preventing generation of impurities associated with the catalyst decomposition. In one specific embodiment, Step 4 is performed at a temperature in a range of 20° C. to 30° C., such 20° C. to 25° C.

In some embodiments, the methods further comprise reacting Compound (B) with an acid to generate Compound (C), as depicted in Step 5 of general scheme 1. Examples of suitable acids include TFA (trifluoroacetic acid) (e.g., TFA (e.g., 3 eq) in MeOH (methanol), acetone, or MTBE (methyl t-butyl ether)), H₂SO₄ (e.g., H₂SO₄ (e.g., 3 eq) in acetone/H₂O), TCA (trichloroacetic acid) (e.g., TCA (e.g., 3 eq) in MeOH or MTBE), H₃PO₄ (e.g., H₃PO₄ (e.g., 3 eq) in MTBE), TMSCl (trimethylsilyl chloride) (e.g., TMSCl (e.g., 3 eq) in MTBE), Amberlyst 15 (e.g, Amberlyst 15 (e.g., 25 mg) in MTBE), HCl (e.g., HCl (e.g., 2 eq, 5 eq, 6.5 eq) in dioxane/acetone, dioxane/acetone/H₂O, or THF/H₂O), ZnCl₂ (e.g., ZnCl₂ in THF and/or H₂O), AlCl₃ (e.g., AlCl₃ in THF/H₂O), NH₄CO₂CF₃ (e.g., NH₄CO₂CF₃ in THF/H₂O), Ce(OTf)₃ (e.g., Ce(OTf)₃ (Tf: triflate) in MeNO₂/H₂O), CuCl₂ (e.g., CuCl₂ in acetonitrile)), FeCl₃ (e.g., FeCl₃ in DCM (dichloromethane)/acetone), tartaric acid (e.g., tartaric acid (e.g. 3 eq) in acetone), and AcOH (acetic acid) (e.g., AcOH (e.g., 3 eq) in acetone). Additional suitable examples include oxalic acid in MeOH, MIBK (methyl isobutyl ketone), 2-butanol (2-BuOH), or 2-butanone. In one specific embodiment, the acid is HCl, such as aqueous HCl. A typical concentration of aqueous HCl which can be employed in Step 5 is in a range of 1N to 6N, such as 1.6N to 3N (e.g., 2N). In one specific embodiment, the aqueous HCl is added to a solution of Compound (B) in acetone and/or 2-butanone maintained at a temperature in a range of 50° C. to 65° C., such as 50° C. to 60° C., or approximately 55° C. In another specific embodiment, the treatment of Compound (B) with HCl includes: i) adding a first aqueous HCl solution to a solution of Compound (B) in 2-butanone; ii) stirring the mixture for at least an hour; iii) adding a second aqueous HCl solution to the resulting mixture of step ii); and iv) stirring the resulting mixture of step iii) for at least an hour. Without being bound to a particular theory, this recharging of a second aqueous HCl solution once the reaction between Compound (B) and the first aqueous HCl solution reaches an equilibrium (e.g., around 96-98% conversion) brings the conversion of Compound (B) to Compound (C) over 99% (e.g., 99.5% conversion). Also, carrying Compound (B) as an impurity over to the next step can be minimized, which can improve the overall purity of Compound (1).

Optionally, if desired, the resulting product of Step 5 can be crystallized from a suitable solvent system. As used herein, the term “crystallization” or “crystallized” includes “recrystallization” or “recrystallized.” In one example, it is crystallized from a mixture of acetone, 2-butanone, and water (e.g., solution or suspension of Compound (C) in acetone, 2-butanone, and water).

The cyclohexanone of Compound (C) can further be reduced to cyclohexanol of Compound (D), as depicted in Step 6 of general scheme 1. Any suitable reducing agent known in the art can be employed for Step 6. Suitable examples include LiAlH(OⁱBu)₂(O^{t}Bu)₃, DiBAlH (diisobutylaluminum hydride), LiBH₄, NaBH₄, NaBH(OAc)₃, Bu₄NBH₄, ADH005 MeOH/KRED recycle mix A, KRED-130 MeOH/KRED recycle mix A, Al(OⁱPr)₃/ⁱPrOH, (ⁱBu)₂AlOⁱPr (^{t}Bu: tert-butyl; ⁱBu: iso-butyl; Me: methyl; Ac: acetyl; ⁱPr: isopropyl). One specific example is LiAlH(O^{t}Bu)₃ wherein ^{t}Bu is ter-butyl. Typically, the reduction is performed at a temperature in a range of −70° C. to −35° C., such as −70° C. to −40° C. or −50° C. to −40° C. In one specific embodiment, LiAlH(O^{t}Bu)₃ is added portion wise into a solution of Compound (C) (e.g., a solution of Compound (C) in THF and/or 2-MeTHF), for example over an hour or 2 hours. Step 6 may further include treating the reaction material resulted from the treatment of Compound (C) with a suitable reducing agent (e.g., LiAlH(O^{t}Bu)₃) with an acid, such as tartaric acid or oxalic acid, or a mixture thereof. In one specific embodiment, the acid is tartaric acid. In another specific embodiment, the acid is oxalic acid. Optionally, if desired, the resulting product of Step 6 can be crystallized (e.g., recrystallization) from a suitable solvent system. In one example, it is crystallized from a mixture of methanol and water (e.g., solution or suspension of Compound (D) in methanol and water).

Without being bound to a particular theory, Step 6 employing LiAlH(O^{t}Bu)₃ can generate over 95% of Compound (D) (e.g., over 97%) (as compared to its cis isomer) in solution prior to isolation. Further isolation of Compound (D) from the solution can generate over 99% of the desired Compound (D) (as compared to its cis isomer).

Compound (D) can be treated with a base to produce Compound (1). Step 7 of general scheme. Examples of suitable bases for Step 7 include NaOH, LiOH, Bu₄NOH, NaOMe, KOH, and KOH/Bu₄NBr, and a combination thereof, wherein Bu is n-butyl and Me is methyl. In one specific embodiment, the base includes NaOH, LiOH, Bu₄NOH, or NaOMe. In another specific embodiment, the base includes NaOH or Bu₄NOH. In a specific embodiment, a THF or Me-THF solution of compound (D) is treated with the base.

In some embodiments, the resulting product of Step 7 can be crystallized (including recrystallization) from a suitable solvent system. As used herein, the term “crystallization” includes recrystallization also. In a specific embodiment, the resulting product of Step 7 can be crystallized to form Form M of Compound (1). In another specific embodiment, it is crystallized from a solvent system that includes isopropanol, ethyl acetate, n-butyl acetate, methyl acetate, acetone, 2-butanone, or heptane, or a combination thereof to form From M of Compound (1). In another specific embodiment, the crystallization (or recrystallization) of Compound (1) to form Form M of Compound (1) is performed in isopropanol; ethyl acetate; n-butyl acetate; a mixture of n-butyl acetate and acetone; a mixture of n-butyl acetate and methyl acetate; acetone; butanone; a mixture of n-butyl acetate and heptane; a mixture of acetone and heptane; or a mixture of ethyl acetate and heptane. In yet another specific embodiment, the crystallization of Compound (1) to form Form M of Compound (1) is performed in ethyl acetate; n-butyl acetate; or a mixture of n-butyl acetate and acetone. In yet another specific embodiment, the crystallization of Compound (1) to form Form M of Compound (1) is performed in isopropanol at a temperature in a range of 10° C. to 47° C. In yet another specific embodiment, the crystallization of Compound (1) to form Form M of Compound (1) is performed in ethyl acetate is stirred at a temperature in a range of 45° C. to 47° C. In yet another specific embodiment, the crystallization of Compound (1) to form Form M of Compound (1) is performed in n-butyl acetate at a temperature in a range of 35° C. to 47° C. In yet another specific embodiment, the crystallization of Compound (1) to form Form M of Compound (1) is performed in a mixture of n-butyl acetate and acetone (e.g., 5 wt %-95 wt % n-butyl acetate and 5 wt %-95 wt % acetone, such as 90 wt % n-butyl acetate and 10 wt % acetone) at a temperature in a range of 30° C. to 47° C. In yet another specific embodiment, the crystallization of Compound (1) to form Form M of Compound (1) is performed in a mixture of n-butyl acetate and methyl acetate (5 wt %-95 wt % n-butyl acetate and 5 wt %-95 wt % methyl acetate, such as 50 wt % n-butyl acetate and 50 wt % methyl acetate) at a temperature in a range of 25° C. to 47° C. In yet another specific embodiment, the crystallization of Compound (1) to form Form M of Compound (1) is performed in acetone at a temperature in a range of 20° C. to 47° C. In yet another specific embodiment, the crystallization of Compound (1) to form Form M of Compound (1) is performed in butanone at a temperature in a range of 30° C. to 47° C. In yet another specific embodiment, the crystallization of Compound (1) to form Form M of Compound (1) is performed in a mixture of n-butyl acetate and heptane (e.g., 5 wt %-95 wt % n-butyl acetate and 5 wt %-95 wt % heptane, such as 50 wt % n-butyl acetate and 50 wt % heptane) at a temperature in a range of 25° C. to 47° C. In yet another specific embodiment, the crystallization of Compound (1) to form Form M of Compound (1) is performed in a mixture of acetone and heptane (e.g., 5 wt %-95 wt % acetone and 5 wt %-95 wt % heptane, such as 50 wt % acetone and 50 wt % heptane) at a temperature in a range of 25° C. to 47° C. In yet another specific embodiment, the crystallization of Compound (1) to form Form M of Compound (1) is performed in a mixture of ethyl acetate and heptane (e.g., 5 wt %-95 wt % ethylacetate and 5 wt %-95 wt % heptane, such as 50 wt % ethyl acetate and 50 wt % heptane) at a temperature in a range of 25° C. to 47° C.

Polymorph Form M of Compound (1) can be characterized by, e.g., its X-ray powder diffraction (XRPD) pattern, thermogravimetric analysis (TGA), differential scanning calorimetry (DSC), and/or solid state C¹³ nuclear magnetic spectroscopy (NMR) spectrum. In one specific embodiment, the polymorphic Form M is characterized as having an X-ray powder diffraction pattern (obtained at room temperature using Cu K alpha radiation) with the most intense characteristic peak expressed in 2-theta±0.2 at 19.6. In another specific embodiment, the polymorphic Form M is characterized as having an X-ray powder diffraction pattern (obtained at room temperature using Cu K alpha radiation) with characteristic peaks expressed in 2-theta±0.2 at the following positions: 19.6, 16.6, 18.1, 9.0, 22.2, and 11.4. In yet another embodiment, the polymorphic Form M is characterized as having an X-ray powder diffraction pattern (obtained at room temperature using Cu K alpha radiation) with characteristic peaks expressed in 2-theta±0.2 at the following positions with relative intensities in parentheses: 19.6 (100.0%), 16.6 (72.4%), 18.1 (59.8%), 9.0 (47.6%), 22.2 (39.9%), and 11.4 (36.6%). In another specific embodiment, the polymorphic Form M is characterized as having an endothermic peak in differential scanning calorimetry (DSC) at 230±2° C. In yet another embodiment, the polymorphic Form M is characterized as having peaks at 177.3, 134.3, 107.4, 56.5, 30.7, and 25.3 in a solid state C¹³ nuclear magnetic spectroscopy (NMR) spectrum.

In some embodiments, the methods of the present invention employ Steps 4-7 of general scheme 1 to prepare Compound (1). Optionally, the methods further include crystallization of Compound (C) from a mixture of acetone and water, or 2-butanone and water (e.g., solution or suspension of Compound (C) in a mixture of acetone and water) prior to Step 6. Optionally, the methods further include crystallization of Compound (D) from a mixture of methanol and water (e.g., solution or suspension of Compound (D) in a mixture of methanol and water) prior to Step 7. The methods optionally further employ crystallization of Compound (1) in ethylacetate (e.g., solution or suspension of Compound (1) in ethylacetate) or in a mixture of n-butylacetate and acetone (e.g., solution or suspension of Compound (1) in n-butylacetate 5 wt %-95 wt % acetone 5 wt %-95 wt %, such as 90 wt % n-butylacetate and 10 wt % acetone).

In some embodiments, the methods of the present invention employ Steps 3-7 of general scheme 1 to prepare Compound (1). As shown in general scheme 1, Compound (A) can be prepared by reacting Compound (E) with I₂ (Step 3). I₂ can be added into a solution of Compound (E) maintained at a temperature in a range of −80° C. to −40° C. (e.g., −78° C. to −40° C., or −50° C. to −40° C.). In a specific embodiment, the reaction of Compound (E) with I₂ is performed in the presence of a base, such as a mixture of ⁱPr₂NH and ⁿBuLi. Features of each of Steps 4-7 are as described above.

In some embodiments, the methods of the present invention employ Steps 2-7 of general scheme 1 to prepare Compound (1). As shown in general scheme 1, Compound (E) can be prepared by reacting Compound (G) with Compound (F) (either as isolated acid chloride (Step 2(b)) or in situ prepared acid chloride) (Step 2(a)). In one specific embodiment, Compound (F) is provided in situ by reacting Compound (H)

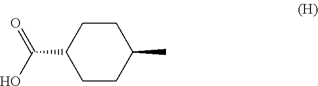

with SOCl₂. In another specific embodiment, Compound (F) is provided in an isolated form. Any suitable condition known in the art for an amidation of an amine with an acid chloride can be employed for Step 2. For example, the amindation can be performed in the presence of a base, such as pyridine. Features of each of Steps 3-7 are as described above.

In some embodiments, the methods of the present invention employ Steps 1-7 of general scheme 1 to prepare Compound (1). As shown in general scheme 1, Compound (G) can be prepared by reacting Compound (J) with Compound (K) (Step 1). Any suitable condition known in the art for an amination of a ketone can be employed for Step 1. For example, Compounds (J) and (K) can be combined with NaBH(OAc)₃ and trichloroacetic acid (where Ac is acetyl). In one specific embodiment, NaBH(OAc)₃ and trichloroacetic acid are combined with Compounds (J) and (K) in Toluene. In a more specific embodiment, trichloroacetic acid in toluene is added to a mixture of Compounds (J), (K), and NaBH(OAc)₃ in toluene. In another more specific embodiment, the mixture of Compounds (J), (K), NaBH(OAc)₃, and trichloroacetic acid in toluene is maintained at a temperature in a range of 20° C. to 25° C. Features of each of Steps 2-7 are as described above.

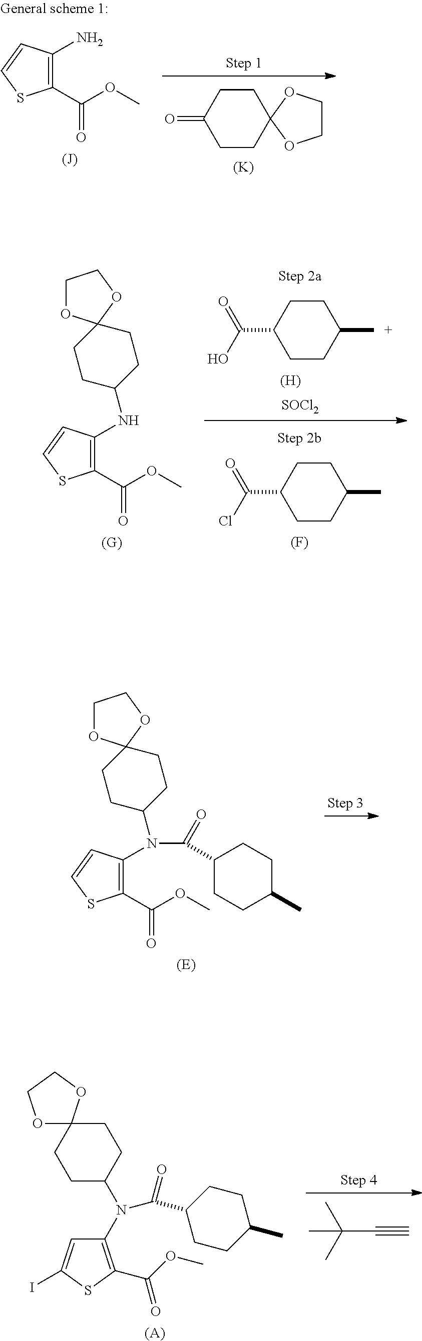

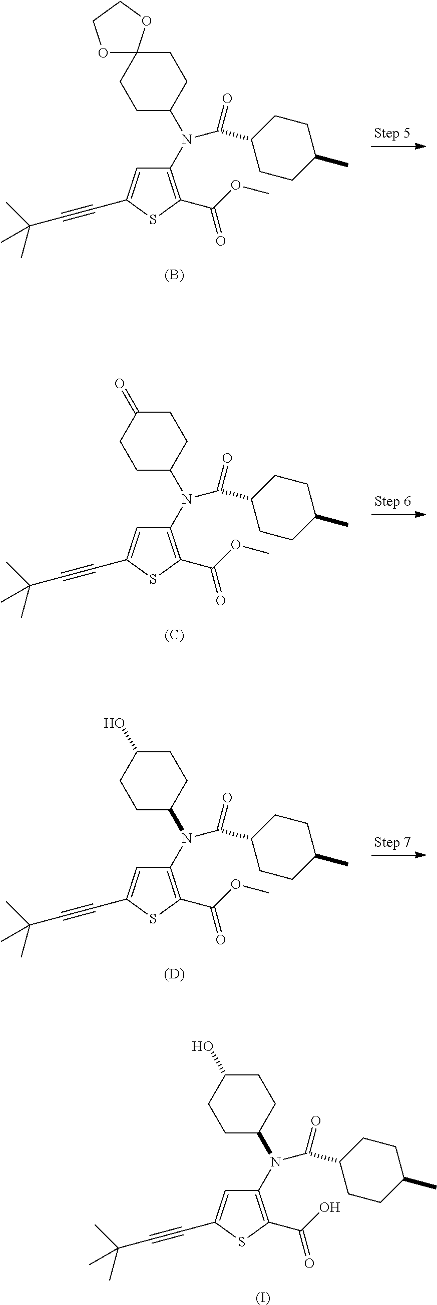

In yet another embodiment, a method of the present invention is directed to a method of preparing Compound (B):

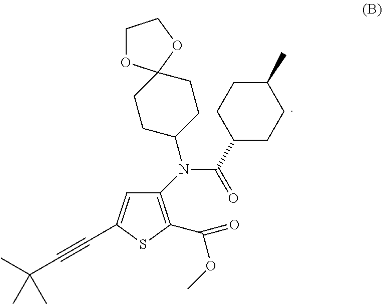

Compound (A) can be reacted with 3,3-dimethylbut-1-yne in the presence of one or more palladium catalysts selected from the group consisting of Pd(PPh₃)₄ and Pd(PPh₃)₂Cl₂, and one or more copper catalysts selected from the group consisting of CuI, CuBr, and CuCl, to generate Compound (B), as depicted in Step 4 of general scheme 1. Features of Step 4 are as described above.

Specific exemplary conditions suitable for each Steps 1-7 of general scheme 1, which each and independently can be employed in the methods of the invention, are described below in the Exemplification section.

The compounds described herein are defined herein by their chemical structures and/or chemical names. Where a compound is referred to by both a chemical structure and a chemical name, and the chemical structure and chemical name conflict, the chemical structure is determinative of the compound's identity.

It will be appreciated by those skilled in the art that in the processes of the present invention certain functional groups such as hydroxyl or amino groups in the starting reagents or intermediate compounds may need to be protected by protecting groups. Thus, the preparation of the compounds described above may involve, at various stages, the addition and removal of one or more protecting groups. The protection and deprotection of functional groups is described in “Protective Groups in Organic Chemistry.” edited by J. W. F. McOmie, Plenum Press (1973) and “Protective Groups in Organic Synthesis,” 3rd edition, T. W. Greene and P. G. M. Wuts, Wiley Interscience, and “Protecting Groups,” 3rd edition, P. J. Kocienski, Thieme (2005)

For purposes of this invention, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausolito: 1999, and “March's Advanced Organic Chemistry”, 5th Ed., Ed.: Smith, M. B. and March, J., John Wiley & Sons, New York: 2001, the entire contents of which are hereby incorporated by reference.

The term “protecting group” and “protective group” as used herein, are interchangeable and refer to an agent used to temporarily block one or more desired functional groups in a compound with multiple reactive sites. In certain embodiments, a protecting group has one or more, or specifically all, of the following characteristics: a) is added selectively to a functional group in good yield to give a protected substrate that is b) stable to reactions occurring at one or more of the other reactive sites; and c) is selectively removable in good yield by reagents that do not attack the regenerated, deprotected functional group. As would be understood by one skilled in the art, in some cases, the reagents do not attack other reactive groups in the compound. In other cases, the reagents may also react with other reactive groups in the compound. Examples of protecting groups are detailed in Greene, T. W., Wuts, P. G in “Protective Groups in Organic Synthesis”, Third Edition, John Wiley & Sons, New York: 1999 (and other editions of the book), the entire contents of which are hereby incorporated by reference. The term “nitrogen protecting group”, as used herein, refers to an agent used to temporarily block one or more desired nitrogen reactive sites in a multifunctional compound. Preferred nitrogen protecting groups also possess the characteristics exemplified for a protecting group above, and certain exemplary nitrogen protecting groups are also detailed in Chapter 7 in Greene, T. W., Wuts, P. G in “Protective Groups in Organic Synthesis”, Third Edition, John Wiley & Sons, New York: 1999, the entire contents of which are hereby incorporated by reference.

As used herein, the term “displaceable moiety” or “leaving group” refers to a group that is associated with an aliphatic or aromatic group as defined herein and is subject to being displaced by nucleophilic attack by a nucleophile.

It will be appreciated by those skilled in the art that the compounds described herein can exist in different polymorphic forms. As known in the art, polymorphism is an ability of a compound to crystallize as more than one distinct crystalline or “polymorphic” species. A polymorph is a solid crystalline phase of a compound with at least two different arrangements or polymorphic forms of that compound molecule in the solid state. Polymorphic forms of any given compound are defined by the same chemical formula or composition and are as distinct in chemical structure as crystalline structures of two different chemical compounds.

Specific examples of polymorphic forms of Compound (1) include Form A, Form M, Form H, and Form P, as shown in the Exemplification section below. Form M of Compound (1) can be prepared by stirring a mixture of Compound (1) and a solvent system that includes isopropanol, ethyl acetate, n-butyl acetate, methyl acetate, acetone, 2-butanone, or heptane, or a combination thereof, as described above for the crystallization (including recrystallization) of Compound (1) to form Form M of Compound (1). Form H of Compound (1) can be prepared by stirring a solution of Compound (1) at a temperature in a range of 48° C. to 70° C. or 50° C. to 70° C. In one specific embodiment, a mixture of Compound (1) and a solvent system that includes ethyl acetate is stirred at a temperature in a range of 48° C. to 70° C. for a period of time to form Form H. In another specific embodiment, a mixture of Compound (1) and a solvent that includes ethyl acetate is stirred at a temperature of 65±2° C. for a period of time to form Form H. Form P of Compound (1) can be prepared by heating a mixture of Compound (1) and a solvent system that includes a solvent selected from the group consisting of dichloromethane, and tetrahydrofuran (THF), and a mixture thereof at room temperature. In one specific embodiment, the mixture of Compound (1) and a solvent system that includes dicholoromethane is stirred at room temperature for a period of time to form Form P.

Other specific examples of polymorphic forms of Compound (1) include Form X and Form ZA, as shown in the Exemplification section below. Form X of Compound (1) can be prepared by de-solvating the EtOAc solvate G of Compound (1), for example, in vacuum at an elevated temperature in a range of 50° C. to 65° C. (e.g., 60° C.) to remove EtOAc. Form X is isostructural with the EtOAc solvate G (see the Exemplification section below). Form ZA of Compound (1) can be prepared by heating the n-BuOAc solvate A of Compound (1) to a temperature in a range of 140° C. to 150° C. (e.g., 145° C.) (see the Exemplification section below).

The methods of the invention can be employed for preparing co-crystals of Compound (1). The term “co-crystal” as used herein means a crystalline material comprised of two or more unique solids at room temperature, each containing distinctive physical characteristics, such as structure, melting point and heats of fusion, with the exception that, if specifically stated, the active pharmaceutical ingredient (API) may be a liquid at room temperature. The co-crystals typically comprise the API and a co-crystal former. The co-crystal former may be H-bonded directly to the API or may be H-bonded to an additional molecule which is bound to the API. Other modes of molecular recognition may also be present including, pi-stacking, guest-host complexation and van der Waals interactions. The additional molecule may be H— bonded to the API or bound ionically or covalently to the API. The additional molecule could also be a different API. Solvates of API compounds that do not further comprise a co-crystal forming compound are not co-crystals according to the present invention. The co-crystals may however, include one or more solvate molecules in the crystalline lattice. That is, solvates of co-crystals, or a co-crystal further comprising a solvent or compound that is a liquid at room temperature, is included in the present invention, but crystalline material comprised of only one solid and one or more liquids (at room temperature) are not included in the present invention, with the previously noted exception of specifically stated liquid APIs.

Specific examples of co-crystals comprising Compound (1) include co-crystals of Compound (1) and a co-crystal former selected from the group consisting of urea, nicotinamide, and isonicotinamide, as shown in the Exemplification below. Such co-crystals can be prepared by employing the step of stirring a mixture of Compound (1) and the co-crystal former (urea, nicotinamide, or isonicotinamide) in a suitable solvent at room temperature for a period of time to form the co-crystal. In a specific embodiment, Compound (1) and the co-crystal former are in a 1:1 molar ratio.

The compounds described herein can exist in free form, or, where appropriate, as salts. Those salts that are pharmaceutically acceptable are of particular interest since they are useful in administering the compounds described above for medical purposes. Salts that are not pharmaceutically acceptable are useful in manufacturing processes, for isolation and purification purposes, and in some instances, for use in separating stereoisomeric forms of the compounds of the invention or intermediates thereof.

As used herein, the term “pharmaceutically acceptable salt” refers to salts of a compound, which are, within the scope of sound medical judgment, suitable for use in humans and lower animals without undue side effects, such as, toxicity, irritation, allergic response and the like, and are commensurate with a reasonable benefit/risk ratio.

Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds described herein include those derived from suitable inorganic and organic acids and bases. These salts can be prepared in situ during the final isolation and purification of the compounds.

Where the compound described herein contains a basic group, or a sufficiently basic bioisostere, acid addition salts can be prepared by, for example, 1) reacting the purified compound in its free-base form with a suitable organic or inorganic acid; and 2) isolating the salt thus formed. In practice, acid addition salts might be a more convenient form for use and use of the salt amounts to use of the free basic form.

Examples of pharmaceutically acceptable, non-toxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, glycolate, gluconate, glycolate, hemisulfate, heptanoate, hexanoate, hydrochloride, hydrobromide, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, palmoate, pectinate, persulfate, 3-phenylpropionate, phosphate, picrate, pivalate, propionate, salicylate, stearate, succinate, sulfate, tartrate, thiocyanate, p-toluenesulfonate, undecanoate, valerate salts, and the like.

Where the compound described herein contains a carboxy group or a sufficiently acidic bioisostere, base addition salts can be prepared by, for example, 1) reacting the purified compound in its acid form with a suitable organic or inorganic base and 2) isolating the salt thus formed. In practice, use of the base addition salt might be more convenient and use of the salt form inherently amounts to use of the free acid form. Salts derived from appropriate bases include alkali metal (e.g., sodium, lithium, and potassium), alkaline earth metal (e.g., magnesium and calcium), ammonium and N(C₁₋₄alkyl)₄⁺ salts. This invention also envisions the quaternization of any basic nitrogen-containing groups of the compounds disclosed herein. Water or oil-soluble or dispersible products may be obtained by such quaternization.

Basic addition salts include pharmaceutically acceptable metal and amine salts. Suitable metal salts include the sodium, potassium, calcium, barium, zinc, magnesium, and aluminium. The sodium and potassium salts are usually preferred. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. Suitable inorganic base addition salts are prepared from metal bases which include sodium hydride, sodium hydroxide, potassium hydroxide, calcium hydroxide, aluminium hydroxide, lithium hydroxide, magnesium hydroxide, zinc hydroxide and the like. Suitable amine base addition salts are prepared from amines which are frequently used in medicinal chemistry because of their low toxicity and acceptability for medical use. Ammonia, ethylenediamine, N-methyl-glucamine, lysine, arginine, ornithine, choline, N,N′-dibenzylethylenediamine, chloroprocaine, dietanolamine, procaine, N-benzylphenethylamine, diethylamine, piperazine, tris(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, dicyclohexylamine and the like.

Other acids and bases, while not in themselves pharmaceutically acceptable, may be employed in the preparation of salts useful as intermediates in obtaining the compounds described herein and their pharmaceutically acceptable acid or base addition salts.

Specific examples of pharmaceutically acceptable salts of Compound (1) are described in WO 2008/058393, such as salts derived from amino acids (e.g. L-arginine, L-Lysine), salts derived from appropriate bases include alkali metals (e.g. sodium, lithium, potassium), alkaline earth metals (e.g. calcium, magnesium), ammonium, NR₄⁺ (where R is C₁₋₄ alkyl) salts, choline and tromethamine salts. In one embodiment, the pharmaceutically acceptable salt is a sodium salt. In another embodiment, the pharmaceutically acceptable salt is a lithium salt. In yet another embodiment, the pharmaceutically acceptable salt is a potassium salt. In yet another embodiment, the pharmaceutically acceptable salt is a tromethamine salt. In yet another embodiment, the pharmaceutically acceptable salt is an L-arginine salt.

It should be understood that this invention includes mixtures/combinations of different pharmaceutically acceptable salts and also mixtures/combinations of compounds in free form and pharmaceutically acceptable salts.

In some embodiments, the compounds in accordance with the present invention are provided as pharmaceutically acceptable salts. As discussed above, such pharmaceutically acceptable salts can be derived from pharmaceutically acceptable inorganic and organic acids and bases. Examples of suitable acids include hydrochloric, hydrobromic, sulphuric, nitric, perchloric, fumaric, maleic, phosphoric, glycollic, lactic, salicylic, succinic, toleune-p-sulphonic, tartaric, acetic, trifluoroacetic, citric, methanesulphonic, formic, benzoic, malonic, naphthalene-2-sulphonic and benzenesulphonic acids. Other acids such as oxalic, while not themselves pharmaceutically acceptable, may be useful as intermediates in obtaining the compounds of the invention and their pharmaceutically acceptable acid addition salts.

Salts derived from amino acids are also included (e.g. L-arginine, L-Lysine).

Salts derived from appropriate bases include alkali metals (e.g. sodium, lithium, potassium), alkaline earth metals (e.g. calcium, magnesium), ammonium, NR₄⁺ (where R is C₁₋₄ alkyl) salts, choline and tromethamine.

In one embodiment of the invention, the pharmaceutically acceptable salt is a sodium salt.

In one embodiment of the invention, the pharmaceutically acceptable salt is a potassium salt.

In one embodiment of the invention, the pharmaceutically acceptable salt is a lithium salt.

In one embodiment of the invention, the pharmaceutically acceptable salt is a tromethamine salt.

In one embodiment of the invention, the pharmaceutically acceptable salt is an L-arginine salt.

In addition to the compounds described herein, the methods of the invention can be employed for preparing pharmaceutically acceptable solvates (e.g., hydrates) and clathrates of these compounds.

As used herein, the term “pharmaceutically acceptable solvate” is a solvate of a compound, which are, within the scope of sound medical judgment, suitable for use in humans and lower animals without undue side effects, such as, toxicity, irritation, allergic response and the like, and are commensurate with a reasonable benefit/risk ratio. The term solvate includes hydrates (e.g., hemihydrate, monohydrate, dihydrate, trihydrate, tetrahydrate, and the like). Specific examples of solvates include hydrates and solvates of organic solvent(s) (e.g., acetone, ethanol, methanol, isopropanol, ethylacetate, 2-methyl THF, or mixtures thereof).

As used herein, the term “hydrate” means a compound described herein or a salt thereof that further includes a stoichiometric or non-stoichiometric amount of water bound by non-covalent intermolecular forces.

As used herein, the term “clathrate” means a compound described herein or a salt thereof in the form of a crystal lattice that contains spaces (e.g., channels) that have a guest molecule (e.g., a solvent or water) trapped within.

In addition to the compounds described herein, the methods of the invention can be employed for preparing pharmaceutically acceptable derivatives or prodrugs of these compounds.

A “pharmaceutically acceptable derivative or prodrug” includes any pharmaceutically acceptable ester, salt of an ester, or other derivative or salt thereof, of a compound described herein, which, upon administration to a recipient, is capable of providing, either directly or indirectly, a compound described herein or an inhibitorily active metabolite or residue thereof. Particularly favoured derivatives or prodrugs are those that increase the bioavailability of the compounds when such compounds are administered to a patient (e.g., by allowing an orally administered compound to be more readily absorbed into the blood) or which enhance delivery of the parent compound to a biological compartment (e.g., the brain or lymphatic system) relative to the parent species.

As used herein and unless otherwise indicated, the term “prodrug” means a derivative of a compound that can hydrolyze, oxidize, or otherwise react under biological conditions (in vitro or in vivo) to provide a compound described herein. Prodrugs may become active upon such reaction under biological conditions, or they may have activity in their unreacted forms. Examples of prodrugs contemplated in this invention include, but are not limited to, analogs or derivatives of compounds of the invention that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include derivatives of compounds described herein that comprise —NO, —NO₂, —ONO, or —ONO₂ moieties. Prodrugs can typically be prepared using well-known methods, such as those described by BURGER′S MEDICINAL CHEMISTRY AND DRUG DISCOVERY (1995) 172-178, 949-982 (Manfred E. Wolff ed., 5th ed).

A “pharmaceutically acceptable derivative” is an adduct or derivative which, upon administration to a patient in need, is capable of providing, directly or indirectly, a compound as otherwise described herein, or a metabolite or residue thereof. Examples of pharmaceutically acceptable derivatives include, but are not limited to, esters and salts of such esters.

Pharmaceutically acceptable prodrugs of the compounds described above include, without limitation, esters, amino acid esters, phosphate esters, metal salts and sulfonate esters.

A pharmaceutically acceptable prodrug can be readily prepared using methods known in the art, such as those described in Burger's Medicinal Chemistry and Drug Chemistry, Vol. 1, 172-178 and 949-982, John Wiley & Sons (1995). See also Bertolini et al., J. Med. Chem., 40, 2011-2016 (1997); Shan et al., J. Pharm. Sci., 86(7), 765-767 (1997); Bagshawe, Drug Dev. Res., 34, 220-230 (1995); Bodor, Advances in Drug Res., 13, 224-331 (1984); Bundgaard, Design of Prodrugs, Elsevier Press (1985); and Larsen, Design and Application of Prodrugs, Drug Design and Development (Krogsgaard-Larsen et al., eds.), Harwood Academic Publishers (1991).

Specific examples of prodrugs of Compound (1) include those described in PCT/US11/42119, filed on Jun. 28, 2011:

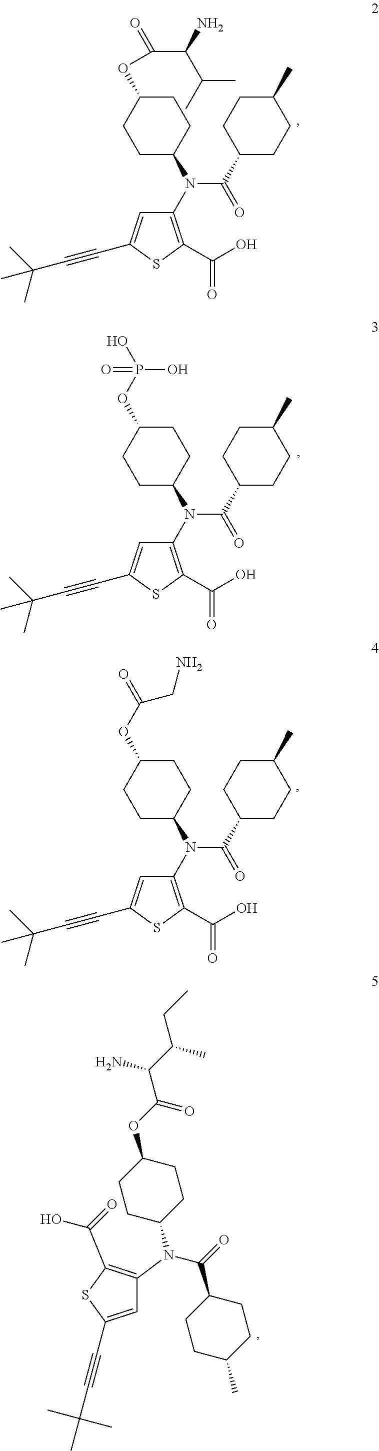

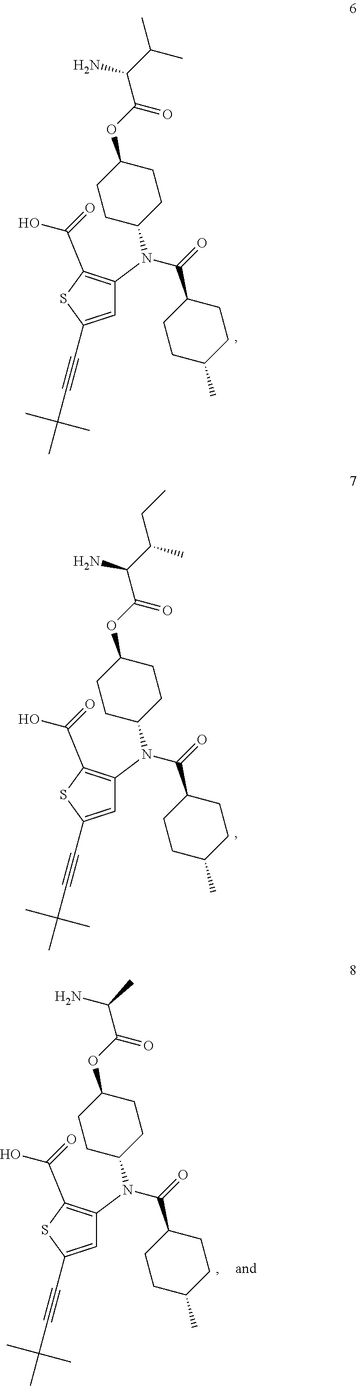 [SMILES: CC(C)[C@@H](N)C(=O)O[C@H]1CC[C@H](N(c2cc(C#CC(C)(C)C)sc2C(=O)O)C(=O)[C@H]2CC[C@H](C)CC2)CC1 ; CC[C@H](C)[C@H](N)C(=O)O[C@H]1CC[C@H](N(c2cc(C#CC(C)(C)C)sc2C(=O)O)C(=O)[C@H]2CC[C@H](C)CC2)CC1 ; C[C@H](N)C(=O)O[C@H]1CC[C@H](N(c2cc(C#CC(C)(C)C)sc2C(=O)O)C(=O)[C@H]2CC[C@H](C)CC2)CC1]

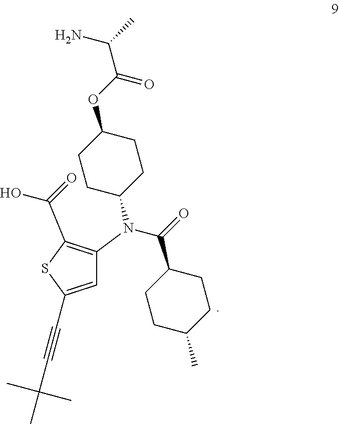 [SMILES: C[C@@H](N)C(=O)O[C@H]1CC[C@H](N(c2cc(C#CC(C)(C)C)sc2C(=O)O)C(=O)[C@H]2CC[C@H](C)CC2)CC1]

It will be appreciated by those skilled in the art that the compounds in accordance with the present invention can exists as stereoisomers (for example, optical (+ and −), geometrical (cis and trans) and conformational isomers (axial and equatorial). All such stereoisomers are included in the scope of the present invention.

Unless otherwise indicated, structures depicted herein are also meant to include all isomeric (e.g., enantiomeric, diastereomeric, cis-trans, conformational, and rotational) forms of the structure. For example, the R and S configurations for each asymmetric center, (Z) and (E) double bond isomers, and (Z) and (E) conformational isomers are included in this invention, unless only one of the isomers is drawn specifically. As would be understood to one skilled in the art, a substituent can freely rotate around any rotatable bonds. For example, a substituent drawn as

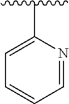 [SMILES: *c1ccccn1]

also represents

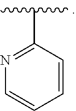 [SMILES: *c1ccccn1]

Therefore, single stereochemical isomers as well as enantiomeric, diastereomeric, cis/trans, conformational, and rotational mixtures of the present compounds are within the scope of the invention.

Unless otherwise indicated, all tautomeric forms of the compounds of the invention are within the scope of the invention.

Additionally, unless otherwise indicated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C— or ¹⁴C-enriched carbon are within the scope of this invention. Such compounds are useful, for example, as analytical tools or probes in biological assays. Such compounds, especially deuterium (D) analogs, can also be therapeutically useful.

It will be appreciated by those skilled in the art that the compounds in accordance with the present invention can contain a chiral center. The compounds of formula may thus exist in the form of two different optical isomers (i.e. (+) or (−) enantiomers). All such enantiomers and mixtures thereof including racemic mixtures are included within the scope of the invention. The single optical isomer or enantiomer can be obtained by method well known in the art, such as chiral HPLC, enzymatic resolution and chiral auxiliary.

In one embodiment, the compounds in accordance with the present invention are provided in the form of a single enantiomer at least 95%, at least 97% and at least 99% free of the corresponding enantiomer.

In a further embodiment, the compounds in accordance with the present invention are in the form of the (+) enantiomer at least 95% free of the corresponding (−) enantiomer.

In a further embodiment, the compounds in accordance with the present invention are in the form of the (+) enantiomer at least 97% free of the corresponding (−) enantiomer.

In a further embodiment, the compounds in accordance with the present invention are in the form of the (+) enantiomer at least 99% free of the corresponding (−) enantiomer.

In a further embodiment, the compounds in accordance with the present invention are in the form of the (−) enantiomer at least 95% free of the corresponding (+) enantiomer.

In a further embodiment, the compounds in accordance with the present invention are in the form of the (−) enantiomer at least 97% free of the corresponding (+) enantiomer.

In a further embodiment the compounds in accordance with the present invention are in the form of the (−) enantiomer at least 99% free of the corresponding (+) enantiomer.

The terms “subject,” “host,” or “patient” includes an animal and a human (e.g., male or female, for example, a child, an adolescent, or an adult). Preferably, the “subject,” “host,” or “patient” is a human.

Compound (1), various polymorphic forms thereof, pharmaceutically acceptable salts thereof, solvates thereof, derivatives or prodrugs thereof, or cocrystals thereof (collectively “the active compounds” hereinafter) can be used for treating or preventing a Flaviviridae viral infection in a host comprising administering to the host a therapeutically effective amount of at least one compound according to the invention described herein.

In one embodiment, the viral infection is chosen from Flavivirus infections. In one embodiment, the Flavivirus infection is Hepatitis C virus (HCV), bovine viral diarrhea virus (BVDV), hog cholera virus, dengue fever virus, Japanese encephalitis virus or yellow fever virus.

In one embodiment, the Flaviviridae viral infection is hepatitis C viral infection (HCV), such as HCV genotype 1, 2, 3, or 4 infections.

In one embodiment, the active compounds can be used for treatment of HCV genotype 1 infection. The HCV can be genotype 1a or genotype 1b.

In one embodiment, the active compounds can be used for treating or preventing a Flaviviridae viral infection in a host comprising administering to the host a therapeutically effective amount of at least one compound according to the invention described herein, and further comprising administering at least one additional agent chosen from viral serine protease inhibitors, viral polymerase inhibitors, viral helicase inhibitors, immunomodulating agents, antioxidant agents, antibacterial agents, therapeutic vaccines, hepatoprotectant agents, antisense agents, inhibitors of HCV NS2/3 protease and inhibitors of internal ribosome entry site (IRES).

In one embodiment, there is provided a method for inhibiting or reducing the activity of viral polymerase in a host comprising administering a therapeutically effective amount of a compound according to the invention described herein.

In one embodiment, there is provided a method for inhibiting or reducing the activity of viral polymerase in a host comprising administering a therapeutically effective amount of a compound according to the invention described herein and further comprising administering one or more viral polymerase inhibitors.

In one embodiment, viral polymerase is a Flaviviridae viral polymerase.

In one embodiment, viral polymerase is a RNA-dependant RNA-polymerase.

In one embodiment, viral polymerase is HCV polymerase.

In one embodiment, viral polymerase is HCV NS5B polymerase.

In treating or preventing one or more conditions/diseases described above, the compounds described above can be formulated in pharmaceutically acceptable formulations that optionally further comprise a pharmaceutically acceptable carrier, adjuvant or vehicle.

A suitable pharmaceutical composition can include the active compound(s) and at least one pharmaceutically acceptable carrier, adjuvant, or vehicle, which includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutically acceptable compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutically acceptable composition, its use is contemplated to be within the scope of this invention. As used herein, the phrase “side effects” encompasses unwanted and adverse effects of a therapy (e.g., a prophylactic or therapeutic agent). Side effects are always unwanted, but unwanted effects are not necessarily adverse. An adverse effect from a therapy (e.g., prophylactic or therapeutic agent) might be harmful or uncomfortable or risky.

A pharmaceutically acceptable carrier may contain inert ingredients which do not unduly inhibit the biological activity of the compounds. The pharmaceutically acceptable carriers should be biocompatible, e.g., non-toxic, non-inflammatory, non-immunogenic or devoid of other undesired reactions or side-effects upon the administration to a subject. Standard pharmaceutical formulation techniques can be employed.

Some examples of materials which can serve as pharmaceutically acceptable carriers include, but are not limited to, ion exchangers, alumina, aluminum stearate, lecithin, serum proteins (such as human serum albumin), buffer substances (such as twin 80, phosphates, glycine, sorbic acid, or potassium sorbate), partial glyceride mixtures of saturated vegetable fatty acids, water, salts or electrolytes (such as protamine sulfate, disodium hydrogen phosphate, potassium hydrogen phosphate, sodium chloride, or zinc salts), colloidal silica, magnesium trisilicate, polyvinyl pyrrolidone, polyacrylates, waxes, polyethylene-polyoxypropylene-block polymers, methylcellulose, hydroxypropyl methylcellulose, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols; such a propylene glycol or polyethylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

The compounds described above, and pharmaceutically acceptable compositions thereof can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. The term “parenteral” as used herein includes, but is not limited to, subcutaneous, intravenous, intramuscular, intra-articular, intra-synovial, intrasternal, intrathecal, intrahepatic, intralesional and intracranial injection or infusion techniques. Specifically, the compositions are administered orally, intraperitoneally or intravenously.

Any orally acceptable dosage form including, but not limited to, capsules, tablets, aqueous suspensions or solutions, can be used for the oral administration. In the case of tablets for oral use, carriers commonly used include, but are not limited to, lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Liquid dosage forms for oral administration include, but are not limited to, pharmaceutically acceptable emulsions, microemulsions, solutions, suspensions, syrups and elixirs. In addition to the active compounds (the compounds described above), the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compounds can also be in microencapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

Sterile injectable forms may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

In order to prolong the effect of the active compounds administered, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

When desired the above described formulations adapted to give sustained release of the active ingredient may be employed.

Compositions for rectal or vaginal administration are specifically suppositories which can be prepared by mixing the active compound with suitable non-irritating excipients or carriers such as cocoa butter, polyethylene glycol or a suppository wax which are solid at ambient temperature but liquid at body temperature and therefore melt in the rectum or vaginal cavity and release the active compound.

Dosage forms for topical or transdermal administration includes ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, eardrops, and eye drops are also contemplated as being within the scope of this invention. Additionally, transdermal patches, which have the added advantage of providing controlled delivery of a compound to the body, can also be used. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Alternatively, the active compounds and pharmaceutically acceptable compositions thereof may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The active compounds and pharmaceutically acceptable compositions thereof can be formulated in unit dosage form. The term “unit dosage form” refers to physically discrete units suitable as unitary dosage for subjects undergoing treatment, with each unit containing a predetermined quantity of active material calculated to produce the desired therapeutic effect, optionally in association with a suitable pharmaceutical carrier. The unit dosage form can be for a single daily dose or one of multiple daily doses (e.g., about 1 to 4 or more times per day). When multiple daily doses are used, the unit dosage form can be the same or different for each dose. The amount of the active compound in a unit dosage form will vary depending upon, for example, the host treated, and the particular mode of administration, for example, from 0.01 mg/kg body weight/day to 100 mg/kg body weight/day.

It will be appreciated that the amount of a compound according to the invention described herein required for use in treatment will vary not only with the particular compound selected but also with the route of administration, the nature of the condition for which treatment is required and the age and condition of the patient and will be ultimately at the discretion of the attendant physician or veterinarian. In general however a suitable dose will be in the range of from about 0.1 to about 750 mg/kg of body weight per day, for example, in the range of 0.5 to 60 mg/kg/day, or, for example, in the range of 1 to 20 mg/kg/day.

The desired dose may conveniently be presented in a single dose or as divided dose administered at appropriate intervals, for example as two, three, four or more doses per day.

The active compound(s) can be formulated as a pharmaceutical composition which further includes one or more additional agents chosen from viral serine protease inhibitors, viral polymerase inhibitors, viral helicase inhibitors, immunomodulating agents, antioxidant agents, antibacterial agents, therapeutic vaccines, hepatoprotectant agents, antisense agent, inhibitors of HCV NS2/3 protease and inhibitors of internal ribosome entry site (IRES). For example, the pharmaceutical composition may include the active compound(s); one or more additional agents select from non-nucleoside HCV polymerase inhibitors (e.g., HCV-796), nucleoside HCV polymerase inhibitors (e.g., R7128, R1626, and R1479), HCV NS3 protease inhibitors (e.g., VX-950/telaprevir and ITMN-191), interferon and ribavirin; and at least one pharmaceutically acceptable carrier or excipient.

The active compound(s) can be employed as a combination therapy in combination with one or more additional agents chosen from viral serine protease inhibitors, viral NS5A inhibitors, viral polymerase inhibitors, viral helicase inhibitors, immunomodulating agents, antioxidant agents, antibacterial agents, therapeutic vaccines, hepatoprotectant agents, antisense agent, inhibitors of HCV NS2/3 protease and inhibitors of internal ribosome entry site (IRES).

The active compounds and additional agent can be administered sequentially. Alternatively, the active compounds and additional agent can be administered simultaneously. The combinations referred to above may conveniently be presented for use in the form of a pharmaceutical formulation and thus pharmaceutical formulations comprising a combination as defined above together with a pharmaceutically acceptable carrier therefore comprise a further aspect of the invention.

The term “viral serine protease inhibitor” as used herein means an agent that is effective to inhibit the function of the viral serine protease including HCV serine protease in a mammal. Inhibitors of HCV serine protease include, for example, those compounds described in WO 99/07733 (Boehringer Ingelheim), WO 99/07734 (Boehringer Ingelheim), WO 00/09558 (Boehringer Ingelheim), WO 00/09543 (Boehringer Ingelheim), WO 00/59929 (Boehringer Ingelheim), WO 02/060926 (BMS), WO 2006039488 (Vertex), WO 2005077969 (Vertex), WO 2005035525 (Vertex), WO 2005028502 (Vertex) WO 2005007681 (Vertex), WO 2004092162 (Vertex), WO 2004092161 (Vertex), WO 2003035060 (Vertex), of WO 03/087092 (Vertex), WO 02/18369 (Vertex), or WO98/17679 (Vertex).

The term “viral polymerase inhibitors” as used herein means an agent that is effective to inhibit the function of a viral polymerase including an HCV polymerase in a mammal. Inhibitors of HCV polymerase include non-nucleosides, for example, those compounds described in: WO 03/010140 (Boehringer Ingelheim), WO 03/026587 (Bristol Myers Squibb); WO 02/100846 A1, WO 02/100851 A2, WO 01/85172 AI (GSK), WO 02/098424 A1 (GSK), WO 00/06529 (Merck), WO 02/06246 A1 (Merck), WO 01/47883 (Japan Tobacco), WO 03/000254 (Japan Tobacco) and EP 1 256 628 A2 (Agouron).

Furthermore other inhibitors of HCV polymerase also include nucleoside analogs, for example, those compounds described in: WO 01/90121 A2 (Idenix), WO 02/069903 A2 (Biocryst Pharmaceuticals Inc.), and WO 02/057287 A2 (Merck/Isis) and WO 02/057425 A2 (Merck/lsis).

The term “viral NS5A inhibitor” as used herein means an agent that is effective to inhibit the function of the viral NS5A protease in a mammal. Inhibitors of HCV NS5A include, for example, those compounds described in WO2010/117635, WO2010/117977, WO2010/117704, WO2010/1200621, WO2010/096302, WO2010/017401, WO2009/102633, WO2009/102568, WO2009/102325, WO2009/102318, WO2009020828, WO2009020825, WO2008144380, WO2008/021936, WO2008/021928, WO2008/021927, WO2006/133326, WO2004/014852, WO2004/014313, WO2010/096777, WO2010/065681, WO2010/065668, WO2010/065674, WO2010/062821, WO2010/099527, WO2010/096462, WO2010/091413, WO2010/094077, WO2010/111483, WO2010/120935, WO2010/126967, WO2010/132538, and WO2010/122162. Specific examples of HCV NS5A inhibitors include: EDP-239 (being developed by Enanta); ACH-2928 (being developed by Achillion); PPI-1301 (being developed by Presido Pharmaceuticals); PPI-461 (being developed by Presido Pharmaceuticals); AZD-7295 (being developed by AstraZeneca); GS-5885 (being developed by Gilead); BMS-824393 (being developed by Bristol-Myers Squibb); BMS-790052 (being developed by Bristol-Myers Squibb)

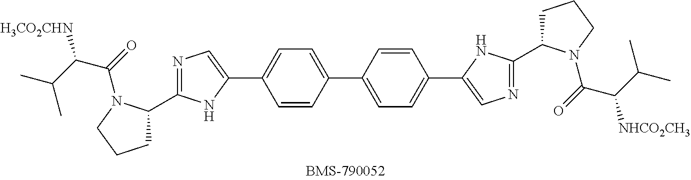

(Gao M. et al. Nature, 465, 96-100 (2010); nucleoside or nucleotide polymerase inhibitors, such as PSI-661 (being developed by Pharmasset), PSI-938 (being developed by Pharmasset), PSI-7977 (being developed by Pharmasset), INX-189 (being developed by Inhibitex), JTK-853 (being developed by Japan Tobacco), TMC-647055 (Tibotec Pharmaceuticals), RO-5303253 (being developed by Hoffmann-La Roche), and IDX-184 (being developed by Idenix Pharmaceuticals).

Specific examples of nucleoside inhibitors of an HCV polymerase, include R1626, R1479 (Roche), R7128 (Roche), MK-0608 (Merck), R1656, (Roche-Pharmasset) and Valopicitabine (Idenix). Specific examples of inhibitors of an HCV polymerase include JTK-002/003 and JTK-109 (Japan Tobacco), HCV-796 (Viropharma), GS-9190 (Gilead), and PF-868,554 (Pfizer).

The term “viral helicase inhibitors” as used herein means an agent that is effective to inhibit the function of a viral helicase including a Flaviviridae helicase in a mammal.

“Immunomodulatory agent” as used herein means those agents that are effective to enhance or potentiate the immune system response in a mammal. Immunomodulatory agents include, for example, class I interferons (such as alpha-, beta-, delta- and omega-interferons, x-interferons, consensus interferons and asialo-interferons), class II interferons (such as gamma-interferons) and pegylated interferons.

Exemplary immunomodulating agents, include, but are not limited to: thalidomide, IL-2, hematopoietins, IMPDH inhibitors, for example Merimepodib (Vertex Pharmaceuticals Inc.), interferon, including natural interferon (such as OMNIFERON, Viragen and SUMIFERON, Sumitomo, a blend of natural interferon's), natural interferon alpha (ALFERON, Hemispherx Biopharma, Inc.), interferon alpha n1 from lymphblastoid cells (WELLFERON, Glaxo Wellcome), oral alpha interferon, Peg-interferon, Peg-interferon alfa 2a (PEGASYS, Roche), recombinant interferon alpha 2a (ROFERON, Roche), inhaled interferon alpha 2b (AERx, Aradigm), Peg-interferon alpha 2b (ALBUFERON, Human Genome Sciences/Novartis, PEGINTRON, Schering), recombinant interferon alfa 2b (INTRON A, Schering), pegylated interferon alfa 2b (PEG-INTRON, Schering, VIRAFERONPEG, Schering), interferon beta-1a (REBIF, Serono, Inc. and Pfizer), consensus interferon alpha (INFERGEN, Valeant Pharmaceutical), interferon gamma-1b (ACTIMMUNE, Intermune, Inc.), un-pegylated interferon alpha, alpha interferon, and its analogs, and synthetic thymosin alpha 1 (ZADAXIN, SciClone Pharmaceuticals Inc.).

The term “class I interferon” as used herein means an interferon selected from a group of interferons that all bind to receptor type 1. This includes both naturally and synthetically produced class I interferons. Examples of class I interferons include alpha-, beta-, delta- and omega-interferons, tau-interferons, consensus interferons and asialo-interferons. The term “class Il interferon” as used herein means an interferon selected from a group of interferons that all bind to receptor type II. Examples of class II interferons include gamma-interferons.

Antisense agents include, for example, ISIS-14803.

Specific examples of inhibitors of HCV NS3 protease, include BILN-2061 (Boehringer Ingelheim) SCH-6 and SCH-503034/Boceprevir (Schering-Plough), VX-950/telaprevir (Vertex) and ITMN-B (InterMune), GS9132 (Gilead), TMC-435350 (Tibotec/Medivir), ITMN-191 (InterMune), and MK-7009 (Merck).

Inhibitor internal ribosome entry site (IRES) includes ISIS-14803 (ISIS Pharmaceuticals) and those compounds described in WO 2006019831 (PTC therapeutics).

In one embodiment, the additional agents for the compositions and combinations include, for example, ribavirin, amantadine, merimepodib, Levovirin, Viramidine, and maxamine.

In one embodiment, the additional agent is interferon alpha, ribavirin, silybum marianum, interleukine-12, amantadine, ribozyme, thymosin, N-acetyl cysteine or cyclosporin.

In one embodiment, the additional agent is interferon alpha 1A, interferon alpha 1 B, interferon alpha 2A, or interferon alpha 2B. Interferon is available in pegylated and non pegylated forms. Pegylated interferons include PEGASYS™ and Peg-intron™.

The recommended dose of PEGASYS™ monotherapy for chronic hepatitis C is 180 mg (1.0 mL vial or 0.5 mL prefilled syringe) once weekly for 48 weeks by subcutaneous administration in the abdomen or thigh.

The recommended dose of PEGASYS™ when used in combination with ribavirin for chronic hepatitis C is 180 mg (1.0 mL vial or 0.5 mL prefilled syringe) once weekly.

Ribavirin is typically administered orally, and tablet forms of ribavirin are currently commercially available. General standard, daily dose of ribavirin tablets (e.g., about 200 mg tablets) is about 800 mg to about 1200 mg. For example, ribavirn tablets are administered at about 1000 mg for subjects weighing less than 75 kg, or at about 1200 mg for subjects weighing more than or equal to 75 kg. Nevertheless, nothing herein limits the methods or combinations of this invention to any specific dosage forms or regime. Typically, ribavirin can be dosed according to the dosage regimens described in its commercial product labels.

The recommended dose of PEG-Intron™ regimen is 1.0 mg/kg/week subcutaneously for one year. The dose should be administered on the same day of the week.

When administered in combination with ribavirin, the recommended dose of PEG-Intron is 1.5 micrograms/kg/week.

The combinations referred to above may conveniently be presented for use in the form of a pharmaceutical formulation and thus pharmaceutical formulations comprising a combination as defined above together with a pharmaceutically acceptable carrier therefore comprise a further aspect of the invention. The individual components for use in the method of the present invention or combinations of the present invention may be administered either sequentially or simultaneously in separate or combined pharmaceutical formulations.

In one embodiment, the additional agent is interferon α 1A, interferon α 1B, interferon α 2A, or interferon α 2B, and optionally ribavirin.

When the active compound(s) is used in combination with at least one second therapeutic agent active against the same virus, the dose of each compound may be either the same as or differ from that when the compound is used alone. Appropriate doses will be readily appreciated by those skilled in the art.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXEMPLIFICATION

### Example 1

### Synthetic Procedures for Steps 1-7

### 1. Step 1

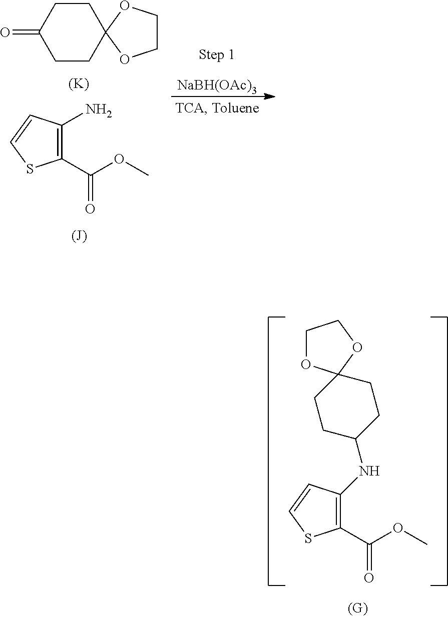

Compounds J (50.0 g, 1.0 eq.), K (52.2 g, 1.05 eq), and NaBH(OAc)₃ (118.0 g, 1.75 eq) were added to a reactor followed by toluene (600 mL, 12 vol). Started agitation then adjusted the internal temperature to 0-5° C. The mixture was a heterogeneous suspension of white solids. Then was added trichloroacetic acid (TCA, 52.0 g, 1.0 eq) in toluene (150 mL, 3 vol) to the stirring mixture over 1 h while controlling the internal temperature to between 0-5° C. The reaction mixture was warmed to 20-25° C., and then stirred for 2-4 hours at 20-25° C. under an atmosphere of nitrogen. The reaction progress was monitored by HPLC.

Upon completion of reaction, the reaction mixture was transferred into a solution of K₂CO₃ (307.7 g, 7.0 eq) in DI water (375 mL, 7.5 vol). The biphasic mixture was stirred and then the phases were separated. The organic phase was washed with aqueous solution of K₂CO₃ (175.9 g, 4.0 eq) in DI water (375 mL, 7.5 vol), then with aqueous solution of NaCl (20.4 g, 1.1 eq) in DI water (375 mL, 7.5 vol). The organic phase was separated. The batch volume was reduced by distillation (to 250 mL (5 vol) on a rotary evaporator at a bath temperature of 40° C.) and the resulting crude solution of Compound G in toluene was used in the next step (HPLC: 98.29% AUC chemical purity). Compound G: ¹H NMR (400 MHz, DMSO-d₆) δ 1.45 (m, 2H), 1.64 (m, 4H), 1.88 (m, 2H), 3.56 (m, 1H), 3.72 (s, 3H), 3.87 (m, 4H), 6.70 (d, J=6.8 Hz, 1H), 6.90 (d, J=4.4 Hz, 1H), 7.70 (d, J=4.4 Hz, 1H).

### 2. Step 2

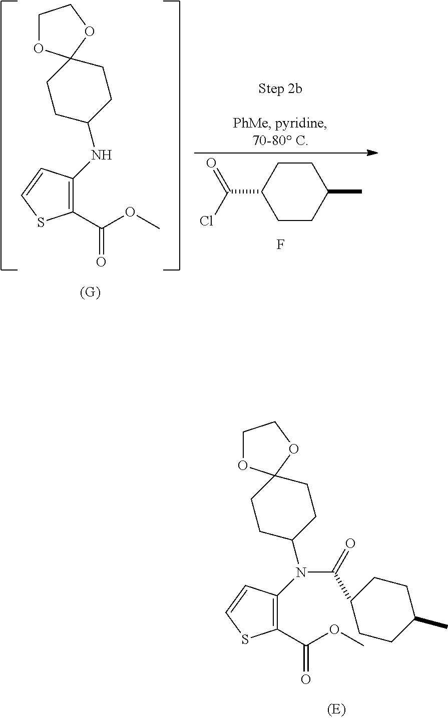

### 2.1. Step 2b: Using Trans-Methylcyclohexane Carbonyl Chloride (Compound F)

To the solution of compound G in toluene (94.6 g, 250 mL, 5.0 vol) from previous step was added toluene (410 mL, 8.2 vol) and pyridine (64.0 mL, 2.5 eq). Agitation was started and the internal temperature was adjusted to 20-25° C. Compound F (102.2 g, 2.0 eq) was added over 0.5 h. The batch was heated to 95-100° C. once the addition had complete. The reaction progress was monitored by HPLC. Upon completion of reaction, the batch was cooled to 30-35° C., then methanol (189 mL, 3.8 vol) was added over 45 minutes and the batch was stirred for 1-2 hours. Added DI water (189 L, 3.8 vol) to the batch at 30-35° C. then it was allowed to stir at 60-70° C. for 1-2 hours. The mixture was heated to 55-60° C. then stirred for 1 h.

The phases were separated. DI water (189 mL, 3.8 vol) was added at 55-60° C. then stirred for 1 hour. The toluene phase was concentrated by distillation. The batch was heated to 78-83° C. (e.g, 80° C.), then n-heptane (473 mL, 9.5 vol) was added to toluene solution over 1-3 hours, and the batch was then stirred at 90-95° C. over 2 hours. The batch was cooled to 20-25° C. over 5 hours, followed by stirring at 20-25° C. for 1-12 hours. The solids were filtered. The filter cake was washed with n-heptane (190 mL, 3.8 vol) and dried under vacuum at 40-45° C. for 10-20 hours. The isolated compound E was analyzed by HPLC, GC, and Karl Fischer titration. Overall yield for Steps 1 & 2=113.5 g, 84.1%. HPLC: 99.39% AUC chemical purity (Typical purity>98.0%). Compound E: ¹H NMR (400 MHz, DMSO-d₆) δ0.48 (m, 1H), 0.63 (m, 1H), 0.74 (d, J=6.4 Hz, 3H), 0.98 (m, 1H), 1.22 (m, 2H), 1.36 (m, 1H), 1.52-1.67 (m, 10H), 1.77 (m, 2H), 3.75-3.78 (m, 4H), 3.76 (s, 3H), 4.44 (m, 1H), 7.11 (d, J=5.2 Hz, 1H), 8.00 (d, J=5.2 Hz, 1H).

### 2.2. Step 2a: Using Trans-Methylcyclohexane Carboxylic Acid (Compound H)

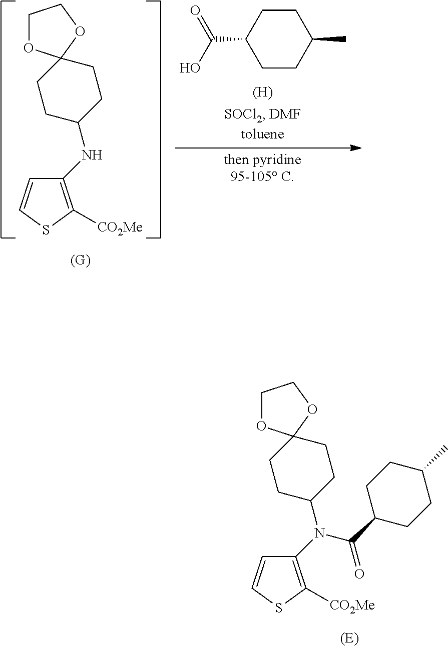

Compound H (633 g, 2.0 eq) was charged to a reactor-1 under a N₂ atmosphere. Toluene (1.33 L, 3.8 vol) was then added to the reactor, followed by DMF (1.73 mL, 0.01 eq), then agitation was started. SOCl₂ (325 mL, 2.0 eq) was added slowly over 30 minutes. The internal temperature was adjusted to 33-37° C. (e.g., 35° C.). The solution was stirred at 33-37° C. for 2 hours. The mixture was cooled to 20-25° C., transferred to a rotary evaporator, and then concentrated to 3.8 vol (˜1.3 L). Toluene (665 mL, 1.9 vol) was then added to the concentrate and the resulting batch was concentrated to 3.8 vol (˜1.3 L).

Compound G in toluene (662 g, 1.75 L, 5.0 vol) was charged to a reactor-2 under N₂ atmosphere. Toluene (4.97 L, 14.2 vol) and pyridine (448 mL, 2.5 eq) was added to the reactor-2. Agitation was started and the internal temperature was adjusted to 20-25° C.

The solution of reactor-1 (acid chloride obtained above) in toluene was added to the reactor-2 over 1 hour. The reaction mixture was heated to 95-105° C. once the addition had complete. An IPC sample was taken after 24-30 h and analyze for Compound G consumption by HPLC.

The reaction mixture was then cooled to 25-30° C. MeOH (665 mL, 1.9 vol) was added to the reaction mixture over 45 minutes. DI water (1.33 L, 3.8 vol) was then added to the reaction mixture at 25-30° C. The mixture was heated to 55-60° C. then stirred for 1 hour. Stopped agitation and allowed the phases to separate for 10 minutes. The upper organic layer was separated and the aqueous layer was set aside. DI water (1.33 L, 3.8 vol) was added to the reaction mixture at 55-60° C. then stirred for 1 hour. Stopped agitation and allowed the phases to separate for 10 minutes. The upper organic layer was separated and the aqueous layer was set aside. The solution was transferred (while it remained at ˜60° C.) to a rotary evaporator and concentrated to 5.7 vol (˜2 L). Heptane (3.3 L, 5.0 vol) was then added to the suspension at ˜60° C. The suspension was cooled to 20-25° C. while stirring over 5 hours. The suspension was filtered. The cake was washed twice with heptane (665 mL, 1.9 vol). The solids were dried on the filter under vacuum. Overall yield for Steps 1 & 2=805.2 g, 85.8% as a white solid. HPLC: 99.15% AUC chemical purity. Compound E: ¹H NMR (400 MHz, DMSO-d₆) δ0.48 (m, 1H), 0.63 (m, 1H), 0.74 (d, J=6.4 Hz, 3H), 0.98 (m, 1H), 1.22 (m, 2H), 1.36 (m, 1H), 1.52-1.67 (m, 10H), 1.77 (m, 2H), 3.75-3.78 (m, 4H), 3.76 (s, 3H), 4.44 (m, 1H), 7.11 (d, J=5.2 Hz, 1H), 8.00 (d, J=5.2 Hz, 1H).

### 3. Step 3

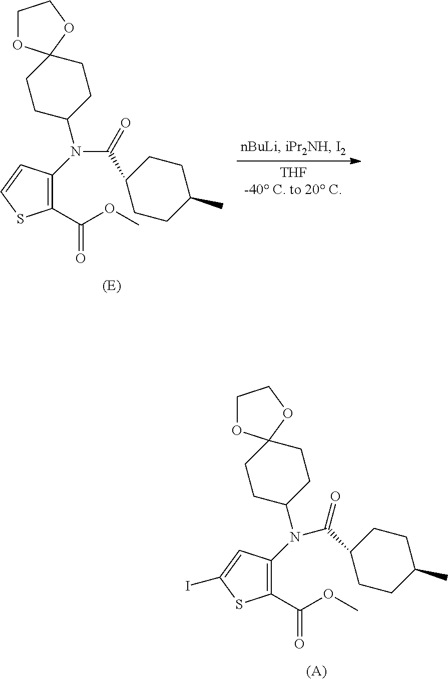

Anhydrous THF (1.0 L, 2.0 vol) and anhydrous diisopropylamine (258 mL, 1.55 eq) were added to Reactor-1. The solution was cooled to −50° C. to −40° C. Once the desired temperature was achieved, a 1.6M solution of n-butyl lithium in hexanes (1.11 L, 1.50 eq) was added at a rate such that the internal temperature remained below −40° C. After the addition had completed, the solution stirred at −50° to −40° C. for another 2 hours.

Compound E (500 g, 1.0 eq) and anhydrous THF (5.0 L, 10.0 vol) were charged to Reactor-2. The resulting solution was added to Reactor-1 over 1 hour at a rate such that the internal temperature remained below −40° C. A solution of iodine (361 g, 1.20 eq) in THF (500 mL, 1.0 vol) was added to the cold reaction mixture at a rate such that the internal temperature remained below −40° C. The reaction mixture was at −50° to −40° C. for 1 hour. The reaction progress was monitored by HPLC.

Upon completion of reaction, the batch was warmed to 0-5° C. and transferred to a solution of NaHSO₃ (617 g, 5.0 eq) in DI water (2.5 L, 5.0 vol) cooled to 0-5° C. Dichloromethane (1.5 L, 3.0 vol) was added to the suspension. The biphasic mixture was stirred for 1 hour while warming to 20-25° C. The phases were separated. The aqueous phase was washed with dichloromethane. The organic phases were combined and washed twice with aqueous solution of NH₄CL (634 g, 10.0 eq) in DI water (1.9 L, 5.0 vol), followed by wash with water. The batch volume was reduced by distillation. Solvent switch to toluene was performed: added toluene (1.5 L, 3.0 vol) again then concentrated to 3.0 vol (˜1.5 L). Toluene (5.0 L, 10.0 vol) was then added to the resulting concentrate and the mixture was heated to 95-100° C. until a homogenous solution was obtained. Added heptane (5.0 L, 10.0 vol) at 95-100° C. to the toluene solution, then the mixture was cooled to 20-25° C. over 6 hours. The suspension was filtered. The cake was washed twice with heptane (500 mL, 1.0 vol). The solids were dried on the filter under vacuum. The isolated compound A was analyzed by HPLC, GC, and Karl Fischer titration. Yield for Steps 3=520.5 g, 80.2% as a beige solid. HPLC: Typical>97.0% AUC chemical purity. Compound A: ¹H NMR (400 MHz, DMSO-d₆) δ 0.54 (m, 1H), 0.65 (m, 1H), 0.76 (d, J=6.8 Hz, 3H), 1.00 (m, 1H), 1.22 (m, 2H), 1.30 (m, 1H), 1.44-1.68 (m, 10H), 1.60-1.69 (m, 4H), 1.77 (m, 2H), 3.74 (s, 3H), 3.77 (m, 4H), 4.40 (m, 1H), 7.46 (s, 1H).

### 4. Step 4

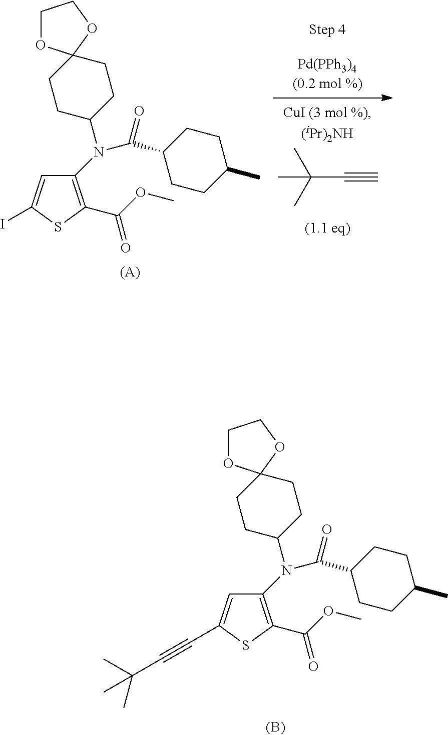

A. Method A1

A jacketed 1 L 3-neck reactor was fitted with a nitrogen inlet then charged with Compound (A) (112.7 g, 205.9 mmol). CuI (1.18 g, 6.18 mmol) and Pd(PPh₃)₄ (457.9 mg, 0.412 mmol) were added to the reactor. The reactor was purged with a stream of nitrogen then anhydrous 2-methyltetrahydrofuran (789 mL) was added. The mixture was stirred for 15 mins at 20-25° C. Anhydrous diisopropylamine (52.09 g, 72.15 mL, 514.8 mmol) and tert-butylacetylene (18.59 g, 27.0 mL, 226.5 mmol) were added to the reactor. This mixture was then stirred between 20-25° C. Complete conversion after stirring for 4 h had been reached according to HPLC. The mixture was cooled to 10° C. The organic phase was then washed with 12.6 wt % aqueous oxalic acid for at least 3 hours then the phases were split. Activated carbon (22.5 g) was added to the reaction mixture. The suspension was stirred at 20-25° C. for not less than 12 hours. The mixture was filtered over celite. The filter cake was washed with 2-butanone (563.5 mL) and the filtrate was added to the organic phase. Analysis of the organic solution by HPLC showed Compound (B) purity to be 99.56% AUC. This solution is typically used directly in the next step. Compound (B): ¹H NMR (400 MHz, DMSO-d₆) δ 0.52-0.59 (m, 1H), 0.61-0.70 (m, 1H), 0.76 (d, J=6.4 Hz, 3H), 0.88-1.03 (m, 1H), 1.15-1.37 (m, 4H), 1.31 (s, 9H) S, 1.41-1.68 (m, 9H), 1.74-1.85 (m, 2H), 3.75-3.81 (m, 4H), 3.75 (s, 3H), 4.39-4.42 (m, 1H), 7.27 (s, 1H).

B. Method A2

A jacketed 1 L 3-neck reactor was fitted with a nitrogen inlet then charged with Compound (A) (63.94 g). CuI (667.3 mg, 0.03 eq) and Pd(PPh₃)₄ (269.9 mg, 0.002 eq) were added to the reactor. The reactor was purged with a stream of nitrogen then methyl t-butyl ether (MtBE) (7 vol) was added. The mixture was stirred for 15 minutes at 20-25° C. Anhydrous diisopropylamine (40.9 mL, 2.5 eq) was added to the stirring mixture while maintaining the internal temperature between 20-25° C. and stirred the batch for NLT 15 minutes. tert-Butylacetylene (16.7 mL, 1.2 eq) were added to the reactor. This mixture was then stirred between 20-25° C. Complete conversion after stirring for 4 h had been reached according to HPLC. The mixture was cooled to 10° C. The organic phase was then washed with 12.6 wt % aqueous oxalic acid dehydrate (383.6 mL, 6 vol) while maintaining the batch temperature below 20-25° C. The batch temperature was then adjusted to 20-25° C. and the biphasic mixture was stirred for at least 3 hours at this temperature. The phases were then allowed to separate for at least 30 minutes. The organic phase was then again washed with aqueous oxalic acid dehydrate (6 wt %, 383.6 mL, 6 vol) while maintaining the batch temperature below 20-25° C. The biphasic mixture was stirred for at least 1 hour at this temperature. Then the phases were split. Activated carbon (6.4 g-12.8 g, 10-20 wt % with respect to Compound A) was added to the reaction mixture. The suspension was stirred at 20-25° C. for not less than 12 hours. The mixture was filtered over celite. The filter cake was washed with MtBE (192 mL, 3 vol) and the filtrate was added to the organic phase. This solution was typically used directly in the next step.

C. Method B

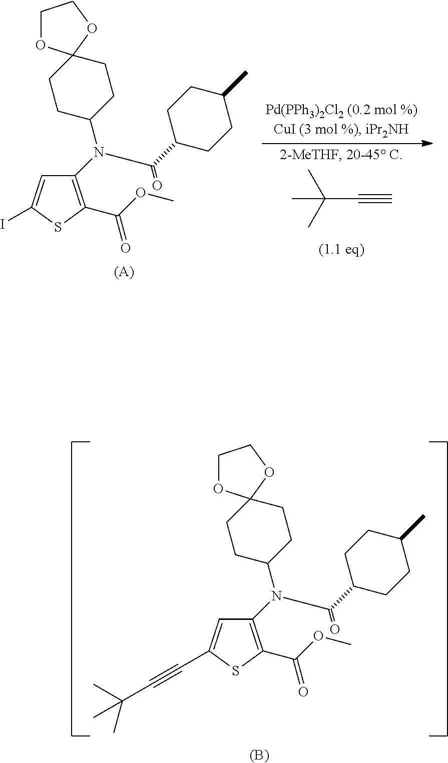

A jacketed 3 L 3-neck reactor was fitted with a nitrogen inlet then charged with Compound (A) (20.00 g, 36.53 mmol). CuI (208.7 mg, 1.096 mmol) and Pd(PPh₃)₂Cl₂ (51.28 mg, 0.07306 mmol) were added to the reactor. The reactor was purged with a stream of nitrogen then anhydrous 2-methyltetrahydrofuran (140.0 mL) was added. The mixture was stirred for 15 mins at 20-25° C. Anhydrous diisopropylamine (9.241 g, 12.80 mL, 91.32 mmol) and tert-butylacetylene (3.751 g, 5.452 mL, 45.66 mmol) were added to the reactor. This mixture was then stirred between 20-25° C. (20.9° C.) (a suspension is formed). The mixture was then heated to 45° C. for 6 h. An HPLC analysis showed conversion to be 99.77%. Heptane (140.0 mL) was added while cooling to 20° C. over 4 h. The suspension was filtered. The filtrate was washed with an aqueous oxalic acid dihydrate solution (120 mL of 15% w/v, 142.8 mmol). The phases were split then the organic phase was washed with aqueous NH₄Cl (120 mL of 10% w/v, 224.3 mmol), aqueous NaHCO₃ (120 mL of 7% w/w), and water (120.0 mL). Residual metals were scavenged by addition of 2.0 g charcoal (10% wt of VRT-0921870) followed by stirring at 20-25° C. for 5 h. The suspension was then filtered over celite. The celite bed was washed with 2-methyltetrahydrofuran (40.0 mL). Analysis of the organic solution by HPLC showed Compound (B) purity to be 99.47% AUC.

D. Method C

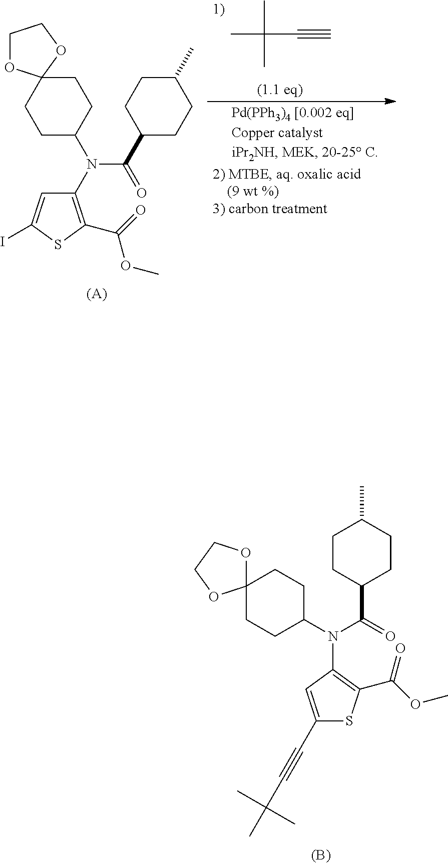

To a round bottom flask equipped with mechanical stirring, N₂ bubbler and thermocouple, was add Compound (A) [1.0 eq], copper catalyst, Pd (PPh₃)_{4 [}0.002 eq] and MEK [7 volume]. The reaction solution was stirred at room temperature to dissolve followed by addition of iPr₂NH [2.5 equiv] and tert-butylacetylene [1.1 equiv]. The reaction solution was stirred at 20-25° C. The reaction conversion was monitored via LC. For the copper catalyst, CuI (99.9%), CuI(98%), CuCl, and CuBr were tested:

CuI (for both 99.9% and 98%): with 0.03 equiv of CuI, over 95% conversion into Compound (B) after about 2 hours' reaction time; with 0.025 equiv of CuI, over 90% conversion into Compound (B) after about 5 hours' reaction time; with 0.02 equiv of CuI, over 90% conversion into Compound (B) after about 5 hours' reaction time; with 0.015 equiv of CuI, over 90% conversion into Compound (B) after about 5 hours' reaction time; with 0.01 equiv of CuI, over 75% conversion into Compound (B) after about 5 hours' reaction time;

CuCl: with 0.03 equiv of CuCl, over 99% conversion into Compound (B) after about 2 hours' reaction time; with 0.025 equiv of CuI, approximately 100% conversion into Compound (B) after about 2 hours' reaction time; with 0.02 equiv of CuCl, over 90% conversion into Compound (B) after about 2 hours' reaction time; with 0.015 equiv of CuCl, over 95% conversion into Compound (B) after about 2 hours' reaction time; with 0.01 equiv of CuCl, approximately 100% conversion into Compound (B) after about 20 hours' reaction time;

CuBr: with 0.03 equiv of CuBr, over 99% conversion into Compound (B) after about 22 hours' reaction time; with 0.025 equiv of CuBr, over 85% conversion into Compound (B) after about 22 hours' reaction time; with 0.02 equiv of CuBr, over 95% conversion into Compound (B) after about 22 hours' reaction time; with 0.015 equiv of CuBr, over 70% conversion into Compound (B) after about 22 hours' reaction time; with 0.01 equiv of CuBr, over 80% conversion into Compound (B) after about 22 hours' reaction time.

### 5. Step 5

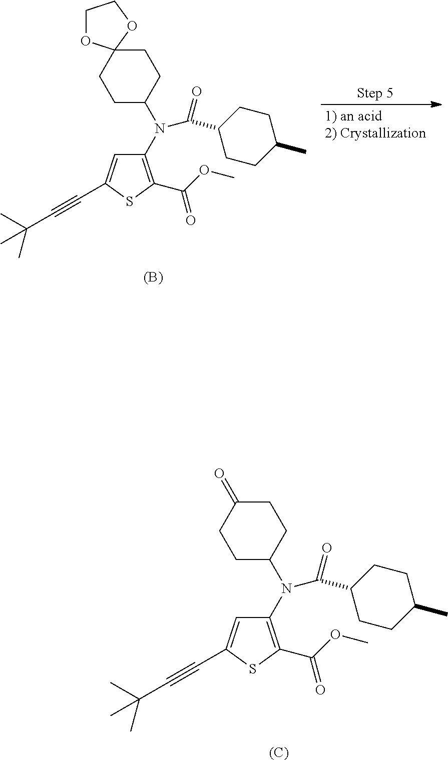

### A. Method A

A jacketed 1 L 4-neck reactor was fitted with a nitrogen inlet then charged with a solution of Compound (B) (22.9 g, 45.65 mmol) in 2-butanone (˜250 mL), then heated to 60° C. The reactor was purged with a stream of nitrogen then an aqueous solution of 2N HCl (175 mL) was added. The mixture was stirred at 60° C. for 4 hours. The stirring was stopped and the lower aqueous phase was removed. Agitation was started again followed by the addition of fresh aqueous solution of 2N HCl (175 mL). The mixture continued to stir at 60° C. until the conversion (99% by HPLC) had reached equilibrium (approximately another 2.5 hours). After cooling to 20° C., the lower aqueous phase was removed. The organic phase was then washed with 10 wt % aqueous NH₄Cl then the phases were split. The organic phase was then distilled to ˜115 mL. Acetone (115 mL) was added then the batch was concentrated to ˜115 mL. This procedure of acetone addition followed by distillation was repeated twice more. Water (57.3 mL) was added to the organic phase at 20° C. then the mixture stirred for 2 hours. Water was added to the organic phase at 20° C. over 2 hours then the mixture stirred for an additional hour. The solids were filtered and washed with 1:1 MeOH/H₂O (25 mL), then dried in a vacuum oven with nitrogen bleed at 60° C. for 24 hours to give 19.8 g (95% yield) of Compound (C). ¹H NMR (400 MHz, DMSO-d₆) δ 0.56-0.68 (m, 2H), 0.76 (d, J=6.4 Hz, 3H), 1.19-1.30 (m, 4H), 1.30 (s, 9H), 1.46-1.60 (m, 6H), 1.83-1.89 (m, 2H), 2.05-2.18 (m, 3H), 2.47-2.55 (m, 1H), 3.76 s, 3H), 4.77-4.85 (m, 1H), 7.30 (s, 1H).

### B. Method B

A jacketed 1 L 4-neck reactor was fitted with a nitrogen inlet then charged with a solution of Compound (B) (103.3 g, 1.0 eq based on 100% yield in Step 4) in 2-butanone (˜1.03 L, approximately 10 vol total batch volume), then heated to 57° C.-62° C. (e.g., 60° C.). The reactor was purged with a stream of nitrogen then an aqueous solution of 2N HCl (723 mL, 7 vol based on 103.3 g of Compound (B)) was added over about 10 minutes while maintaining the batch temperature at 57° C.-62° C. (e.g., 60° C.). The mixture was stirred at 57° C.-62° C. (e.g., 60° C.) for 5 hours. The stirring was stopped and the lower aqueous phase was removed. Agitation was started again followed by the addition of fresh aqueous solution of 2N HCl (310 mL, 3 vol based on 103.3 g of Compound (B)). The mixture continued to stir at 57° C.-62° C. (e.g., 60° C.) until the conversion (99% by HPLC) had reached equilibrium (approximately another 2.5 hours). After cooling to 20-25° C., the agitation was stopped and phases were allowed to separate for at least 30 minutes. An aqueous NH₄Cl (10 wt %, 517 mL, 5 vol) was then added while maintaining the batch temperature at 20-25° C. The biphasic mixture was stirred for at least 30 minutes at 20-25° C. Then the phases were split. The organic phase was then distilled to 471 mL by vacuum distillation with a maximum jacketed temperature of 60° C. Acetone (471.1 mL) was added then the batch was concentrated to ˜471 mL. This procedure of acetone addition followed by distillation was repeated twice more. Water (235.6 mL, 2.28 vol) was added to the organic phase at 20° C. then the mixture stirred for 2 hours. Additional water (235.6 mL, 2.28 vol) was added to the organic phase at 20° C. over 2 hours then the mixture stirred for an additional hour. The solids were filtered and washed with a 1:1 mixture of acetone/H₂O (vol:vol, 103 mL: 103 mL), then dried in a vacuum oven with nitrogen bleed at 60° C. for 24 hours to give 19.8 g (99.5% yield) with overall purity of 98.0%) of Compound (C).

### C. Method C

Aqueous HCl solutions were used in methods A and B above for step 5. Other acids than aqueous HCl could also be used. A summary of the tested acids and conversion (%) is summarized below:

TABLE A
Various Acids for Step 5

| | Temp | Time | Conv | % Com- |
|---|---|---|---|---|
| Conditions | (° C.) | (h) | (%) | pound C |
| TFA (3 eq) in MeOH | 50 | 25.5 | 78.60 | 52.19 |
| TCA (3 eq) in MeOH | 50 | 25.5 | 83.63 | 68.31 |
| conc H₂SO₄ + 10 vol H₂O | 50 | 25.5 | 15.24 | 11.40 |
| H₂SO₄ (3 eq) in acetone | 50 | 25.5 | 84.92 | 37.45 |
| TFA (3 eq) in MTBE | 50 | 21.25 | 9.10 | 7.56 |
| TCA (3 eq) in MTBE | 50 | 21.25 | 8.62 | 6.86 |
| H₂SO₄ (3 eq) in MTBE | 50 | 21.25 | 87.68 | 81.91 |
| H₃PO₄ (3 eq) in MTBE | 50 | 21.25 | 85.70 | 83.61 |
| TMSCl (3 eq) in MTBE | 50 | 21.25 | 5.36 | 2.89 |
| Amberlyst 15 (250 mg) in MTBE | 50 | 21.25 | 63.56 | 61.35 |
| HCl (6.3 eq) in dioxane/acetone | 60 | 30 | 75.70 | 45.92 |
| HCl (6.3 eq) in dioxane/acetone/water | 60 | 30 | 88.79 | 43.48 |
| 2N HCl (5 eq) in MEK | 60 | 30 | 97.95 | 91.14 |
| ZnCl₂ in THF/H₂O | 65 | 7 | 7.22 | 6.02 |
| ZnCl₂/I₂ in THF/H₂O | 65 | 6.75 | 95.80 | 78.65 |
| ZnCl₂/HCl in THF/H₂O | 65 | 6.75 | 92.91 | 90.82 |
| AlCl₃/I₂ in THF/H₂O | 65 | 6.75 | 95.15 | 79.51 |
| AlCl₃/HCl in THF/H₂O | 65 | 6.75 | 93.03 | 90.98 |
| NH₄CO₂CF₃ in THF/H₂O | 65 | 6.75 | 2.54 | 0.65 |
| ZnCl₂/NaI in THF/H₂O | 65 | 98 | 1.37 | 0.20 |
| AlCl₃/NaI in THF/H₂O | 65 | 98 | 78.46 | 77.21 |
| Ce(OTf)₃ in MeNO₂/H₂O | 25 | 98 | 86.84 | 85.50 |
| CuCl₂ 2H₂O in ACN | 65 | 98 | 76.90 | 30.82 |
| Fe(NO₃)₃ 6H₂O in DCM/Acetone | 25 | 98 | 91.86 | 4.28 |
| CuCl₂ 2H₂O in ACN | 25 | 25.66 | 72.26 | 62.54 |
| FeCl₃ 6H₂O in DCM/Acetone | 25 | 25.66 | 86.27 | 80.16 |
| Tartaric acid (3 eq) in acetone | 50 | 27 | 7.26 | 4.60 |
| Oxalic acid (3 eq) in acetone | 50 | 27 | 67.52 | 64.94 |
| AcOH (3 eq) in acetone | 50 | 27 | 3.47 | 0.63 |
| TFA (3 eq) in acetone | 50 | 27 | 69.24 | 66.32 |
| TCA (3 eq) in acetone | 50 | 27 | 70.56 | 65.25 |

TABLE B
Equivalent Screen of Oxalic Acid for Step 5 at ~95°

| Oxalic acid | solvent | conversion | Time |
|---|---|---|---|
| [eq] | [vol] | [%] | [h] |
| 1 | 2-butanol | 98.8 | 2.23 |
| | [7] | | |
| 0.1 | 2-butanol | 94.9 | 2.23 |
| | [7] | | |
| 2.4 | 2-butanone | 35.34 | 4.76 |
| | [7] | | |
| 0.1 | 2-butanone | 98.1 | 4.76 |
| | [7] | | |

### 6. Step 6

### A. Method A: Using LiAlH(OtBu)₃

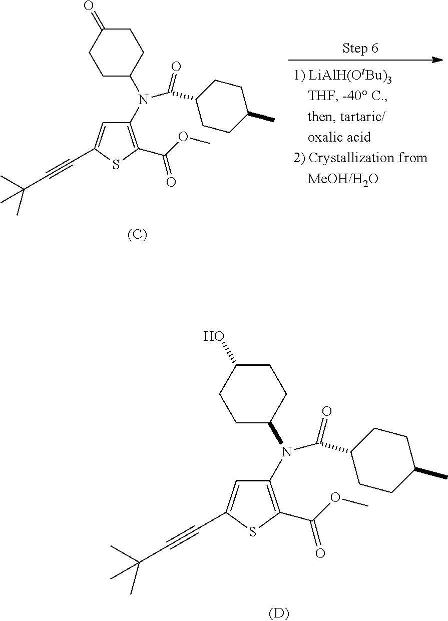

Compound (C) (399 g, 1.0 eq, limiting reagent) was charged to a 12 L reactor and purged with N₂. Anhydrous THF (2 L, 5.0 vol) was then charged to the reactor, then the mixture was agitated. The resulting solution was cooled to −65 to −64° C.

LiAlH(OtBu)₃ (960 ml of 1 M in THF, 2.40 vol or 1.1 eq) was added while maintaining not higher than −40° C. batch temperature. The solution was added over 2 hours and 15 minutes. The rate of addition was 1.45 vol/h.

Upon completion of LiAlH(OtBu)₃ addition, the batch was stirred at −40° C. or lower temperature for 1 additional hour. A small IPC sample was collected after 1 h and immediately quenched with 1 N HCl. The sample was analyzed for Compound (C) consumption (the reaction was judged complete when Compound (C) was ≦0.5% with respect to Compound (D) by IPC method). If reaction was not completed, stir reaction at −40° C. for an additional hour. An IPC sample was collected and immediately quenched with 1 N HCl. If reaction was not completed, then additional amount of LiAlH(OtBu)₃ was added (for instance, if 1.0% peak area of unreacted Compound (C) remained compared to product Compound (D), then 2% of the original charge of LiAlH(OtBu)₃ solution was added). The batch was kept at −40 to −50° C. or lower temperature during reaction. Upon addition of LiAlH(OtBu)₃, the batch was stirred for 1 hour at −45 to −40° C. A small IPC sample was collected and immediately quenched with 1 N HCl. Once the reaction was complete, MTBE (1197 L, 3 vol) was charged to the batch, then the batch was warmed to 0° C. The resulting solution was added over about 10-15 minutes to a mixture of aqueous oxalic acid (or tartaric acid) which was prepared by cooling a mixture of oxalic acid (or tartaric acid) (9% w/w, 2394 L, 6 vol) and MTBE (7 L, 2 vol) to 8-10° C. The batch temperature was adjusted to 15-25° C. and the resulting mixture was stirred for 30-60 minutes.

The agitation was stopped. The upper organic phase was collected. Water (2.8 L, 7 vol) was added to the organic phase. The biphasic mixture was stirred for 10 minutes at 15-25° C. Then agitation was stopped. The upper organic phase was collected.

Crystallization of Compound (D) was performed by switching solvent to methanol. The batch volume was reduced to 1.2 L or 3.0 vol by vacuum distillation at <60° C.

Methanol (4 L, 10 vol) was added to the batch (without adjusting batch temperature) and the batch volume was reduced to 1.2 L or 3.0 vol by vacuum distillation at <60° C. This step was repeated. Then, the batch volume was adjusted to 3.0 vol by addition of 479 mL.

A small IPC sample of the slurry was collected. The solids were filtered and the solution was analyzed by gas chromatography to determine the level of residual THF and MTBE with respect to methanol. If solvent switch to methanol was complete, then the batch was heated to 60-65° C. and stirred at this temperature until all solids dissolved. 2 volumes of the 50 vol % methanol/water solution was added, maintaining the temperature at not less than (NLT) 50° C. Then, the temperature was adjusted to 47-53° C. (e.g., 50° C.), and the temperature was maintained for 4 hours in order for solids to start crystallizing. Then, the remaining 2 volumes of the 50 vol % methanol/water solution were added into the batch. The batch was then cooled 15-25° C. at approximately 5° C./hour, and was held for not less than (NLT) 4 hours at 15-25° C. The filter cake was washed with 1 volume (based on compound 5 charge) of 50 volume % methanol/water

The material was dried for at least 12 hours under vacuum with nitrogen bleed at 55-65° C.

If required, the batch could be recrystallized by charging dry Compound (D) (1 equiv) and methanol (2 vol, relative to Compound (D) charge) to a reactor and heating the batch to 60-65° C. until all solids dissolved. The batch would then be cooled to −20° C. over a 3 hour period. The resulting solids would be filtered and dried for at least 12 hours under vacuum with nitrogen bleed at 55-65° C. Compound D: ¹H NMR (400 MHz, DMSO-d₆) δ 0.52-0.69 (m, 2H), 0.75 (d, 6.4 Hz, 3H), 0.76-0.86 (m, 1H), 1.11-1.24 (m, 5H), 1.31 (s, 9H), 1.43-1.57 (m, 6H), 1.73-1.83 (m, 4H), 3.17-3.18 (m, 1H), 3.75 (s, 3H), 4.24-4.30 (m, 1H), 4.49 (d, J=4.4 Hz, 1H), 7.23 (s, 1H).

B. Method B: Reducing Reagents Other than LiAlH(OtBu)₃

Reducing reagents other than LiAlH(OtBu)₃ that gave predominantly the desired isomer were: LiAlH(OiBu)₂(OtBu)₃, DiBAlH, LiBH4, NaBH4, NaBH(OAc)₃, Bu₄NBH₄, ADH005 MeOH/KRED recycle mix A, KRED-130 MeOH/KRED recycle mix A, Al(Oi-Pr)₃/i-PrOH, and (i-Bu)₂AlOiPr.

### 7. Step 7

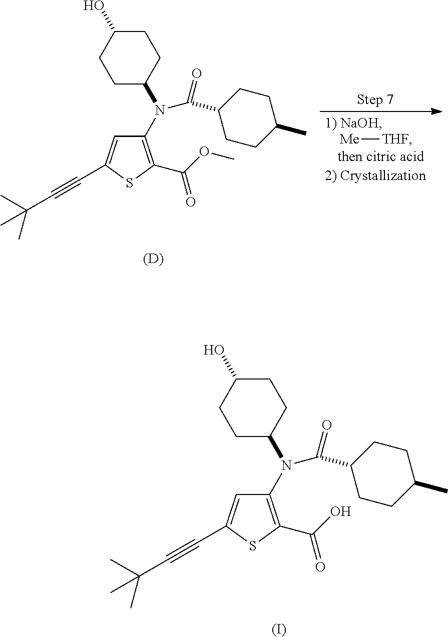

Compound (D) and Me-THF (5 volumes, based on compound 6 charge) were added to a reactor. To the solution, an aqueous solution of NaOH (2N, 4.0 vol, 3.7 equiv) was added at 15-25° C. The batch was heated to 68-72° C. and stirred for 8-16 hours at this temperature. The reaction progress was monitored by LC. Upon completion, the batch was cooled to 0-5° C. Precipitates formed. An aqueous solution of citric acid (30% by weight, 3.7 equiv), was added over 15-30 minutes, while maintaining the batch temperature below 25° C. The phases were separated. Water was added (5 volumes based on compound 6 charge) to the organic layer. The phases were separated. The batch volume was reduced to 3 volumes (based on compound (D) charge) via vacuum distillation at a maximum temperature of 35° C. Then dry Me-THF (3 vol, based on compound (D) charge) was added. The water content was determined by Karl Fisher titration. The batch is deemed dry if residual water level is ≦1.0%.

Optionally, the final product of Compound (1) can be recrystallized either in EtOAc or in a mixture of nBuOAc and acetone via solvent switch described below to form Form M of Compound (1):

A: Recrystallization in a Mixture of nBuOAc and Acetone:

A solvent switch from 2-Me-THF to nBuOAc was performed by first reducing the batch volume to 2-3 volumes (based on compound (D) charge) by vacuum distillation at a maximum temperature of 45° C. nBuOAc (3 vol, based on compound (D) charge) was added and the batch volume was reduced to 2-3 volumes (based on compound (D) charge) via vacuum distillation at a maximum temperature of 45° C. The batch volume was then adjusted to a total of 5-6 volumes by addition of nBuOAc. The solution was analyzed for residual 2-Me-THF in content in nBuOAc. This cycle was repeated until less than 1% of 2-Me-THF with respect to nBuOAc remained, as determined by GC analysis. Once the residual 2-Me-THF IPC criterion was met and it was insured that the total batch volume is 6 (based on compound (D) charge), the batch temperature was adjusted to 40-45° C. Acetone is then charged into the batch to have approximately 10 wt % acetone in the solvent. The batch temperature was adjusted to 40-45° C. Compound 1 seed (1.0% by weight with respect to the total target weight of compound (1)) was added. The batch was agitated at 40-45° C. for 4-8 hours. The recrystallization progress is monitored by X-ray powder diffraction (XRPD). If spectrogram matched that of required form, then the batch was cooled from 40-45° C. to 30-35° C. (preferably about 35° C.) at rate of 5° C./hour. The batch was held at about 35° C. for at least one hour, and then filtered and the filter cake was washed with 9:1 wt:wt mixture of nBuOAc/acetone (1 vol). The material was dried in vacuum with nitrogen bleed at NMT 45° C. for 12-24 hours. The expected isolated molar yield of compound (1) (Form M) starting with compound (D) was 80-85%. Compound (1): ¹H NMR (400 MHz, DMSO-d₆) δ0.58 (m, 1H), 0.74 (q, J=6.53 Hz, 1H), 0.81 (ddd, J=12.86, 12.49, 3.19 Hz, 1H), 1.18 (m, 5H), 1.28 (s, 3H), 1.42 (m, 1H), 1.55 (m, 3H), 1.61 (m, 1H), 1.73 (m, 2H), 1.81 (m, 2H), 3.19 (m, 1H), 4.26 (m, 1H), 4.49 (bs, 1H), 7.14 (s, 1H), 13.45 (bs, 1H).

### B: Recrystallization in EtOAc:

A solvent switch from 2-Me-THF to EtOAc was performed by first reducing the batch volume to 2-3 volumes (based on compound (D) charge) by vacuum distillation at a maximum temperature of 35° C. EtOAc (10 vol, based on compound (D) charge) was added and the batch volume was reduced to 2-3 volumes (based on compound (D) charge) via vacuum distillation at a maximum temperature of 35° C. The solution was analyzed for residual 2-Me-THF in content in EtOAc. This cycle was repeated until less than 1% of Me-THF with respect to EtOAc remained, as determined by GC analysis. Once the residual 2-Me-THF IPC criterion was met and it was insured that the total batch volume is 10 (based on compound (D) charge), the batch temperature was adjusted to 40-45° C. Compound 1 seed (1.0% by weight with respect to the total target weight of compound (1)) was added. The batch was agitated at 40-45° C. for 12 hours. A flat floor/flat bottomed reactor (not conical) should be used. The recrystallization progress is monitored by X-ray powder diffraction (XRPD). If spectrogram matched that of required form, then the batch was cooled from 40-45° C. to 11-14° C. at rate of 5° C./hour. The batch was filtered and the filter cake was washed with EtOAc (1 vol), previously chilled to 11-14° C. The material was dried in vacuum with nitrogen bleed at NMT 45° C. for 12-24 hours. The expected isolated molar yield of compound (1) (Form M) starting with compound (D) was 80-85%.

### Example 2

### Formation of Polymorphic Forms of Compound (1)

### 2A: Formation of Polymorphic Form A of Compound (1)

Polymorphic Form A of Compound (1) can be prepared by following the steps described below:

10 g of Compound (1) prepared according to the procedures depicted in Example 2 was charged to a reactor. 20 g of methanol was then charged to the reactor. The reactor was heated to 60° C. to dissolve Compound (1). The reactor was then cooled to 10° C., and left until solids of Compound (1) formed. The solids of Compound (1) were filtered. 20 g of acetone at 25° C. was added to the solids of Compound (1). The mixture of acetone and Compound (1) was stirred for 1 hour and the resulting solids were filtered. The filtered solids were dried at 75° C. for 12 hours.

Certain representative XRPD peaks and DSC endotherm (° C.) of Form A of Compound (1) are summarized in Table 1 below.

TABLE 1
Certain representative XRPD Peaks
and DSC Endotherm of Form A

| | Form A |
|---|---|
| DSC Endotherm (° C.) | 188° C. |

| XRPD Peaks | Angle (2-Theta ± 0.2) | Intensity % |
|---|---|---|
| 1 | 6.9 | 100.0 |
| 2 | 16.6 | 53.3 |
| 3 | 21.7 | 31.6 |
| 4 | 8.6 | 31.3 |
| 5 | 11.6 | 26.2 |
| 6 | 19.4 | 23.8 |

### 2B: Formation of Polymorphic Form M of Compound (1)

Polymorphic Form M of Compound (1) can be prepared by following the steps described below:

10 g of Compound (1) prepared according to the procedures depicted in Example 2 was charged to a reactor. 50 g of ethyl acetate was then charged to the reactor. The reactor was heated to 45° C. and the mixture was stirred for 1-2 days until Form M was observed. Then, the reactor was cooled to 25° C., and left until solids of Compound (1) formed. The solids of Compound (1) were filtered and the filtered solids were dried at 35° C. for 24 hours.

Alternatively, polymorphic Form M of Compound (1) can be prepared in the following conditions:

| Solvents | Temperature |
|---|---|
| n-BuOAc | 35-47° C. |
| n-BuOAc/Acetone (90%/10%, w/w) | 30-47° C. |
| n-BuOAc/MeOAc (50%/50%, w/w) | 25-47° C. |
| Acetone | 20-47° C. |
| MEK | 30-47° C. |
| n-BuOAc/Heptane (50%/50%, w/w) | 25-47° C. |
| Acetone/Heptane (50%/50%, w/w) | 25-47° C. |
| EtOAc/Heptane (50%/50%, w/w) | 25-47° C. |
| EtOAc | 45-47° C. |

Certain representative XRPD peaks and DSC endotherm (° C.) of Form M of Compound (1) are summarized in Table 2 below.

TABLE 2
Certain representative XRPD Peaks
and DSC Endotherm of Form M

| | Form M |
|---|---|
| DSC Endotherm (° C.) | 230° C. |

| XRPD Peaks | Angle (2-Theta ± 0.2) | Intensity % |
|---|---|---|
| 1 | 19.6 | 100.0 |
| 2 | 16.6 | 72.4 |
| 3 | 18.1 | 59.8 |
| 4 | 9.0 | 47.6 |
| 5 | 22.2 | 39.9 |
| 6 | 11.4 | 36.6 |

### 2C: Formation of Polymorphic Form H of Compound (1)

Polymorphic Form H of Compound (1) can be prepared by following the steps described below:

10 g of Compound (1) prepared according to the procedures depicted in Example 2 was charged to a reactor. 50 g of ethyl acetate was then charged to the reactor. The reactor was heated to 65° C. and the mixture was stirred for 1-2 days until Form H was observed. If desired, a seed(s) of Form H could be added into the reactor for a large scale production. Then, the reactor was cooled to 25° C., and left until solids of Compound (1) formed. The solids of Compound (1) were filtered and the filtered solids were dried at 65° C. for 24 hours.

Certain representative XRPD peaks and DSC endotherm (° C.) of Form H of Compound (1) are summarized in Table 3 below.

TABLE 3
Certain representative XRPD Peaks
and DSC Endotherm of Form H

| | Form H |
|---|---|
| DSC Endotherm (° C.) | 238° C. |

| XRPD Peaks | Angle (2-Theta ± 0.2) | Intensity % |
|---|---|---|
| 1 | 6.6 | 100.0 |
| 2 | 18.7 | 87.8 |
| 3 | 8.5 | 66.7 |
| 4 | 17.3 | 58.4 |
| 5 | 15.8 | 39.9 |
| 6 | 19.4 | 29.8 |

### 2D: Formation of Polymorphic Form P of Compound (1)

Polymorphic Form P of Compound (1) can be prepared by following the steps described below:

### Method A:

20 mg of Compound (1) prepared according to the procedures depicted in Example 2 was charged to a vial. 0.5 mL of dicholormethane was then charged to the vial. The mixture was stirred at room temperature for 3 weeks until solids of Compound (1) were formed. The solids of Compound (1) were filtered and the filtered solids were dried at room temperature for 1 hour.

### Method B:

500 mg of Compound (1) prepared according to the procedures depicted in Example 2 was charged to a vial. 6 mL of dicholormethane was then charged to the vial. The mixture was stirred at room temperature for 4 days until solids of Compound (1) were formed. The solids of Compound (1) were filtered and the filtered solids were dried at room temperature for 1 hour.

Certain representative XRPD peaks and DSC endotherm (° C.) of Form P of Compound (1) are summarized in Table 4A below.

TABLE 4A
Certain representative XRPD Peaks
and DSC Endotherm of Form P

| | Form P |
|---|---|
| DSC Endotherm (° C.) | 160° C. |

| XRPD Peaks | Angle (2-Theta ± 0.2) | Intensity % |
|---|---|---|
| 1 | 7.0 | 100.0 |
| 2 | 15.8 | 21.9 |
| 3 | 9.8 | 14.6 |
| 4 | 19.3 | 11.9 |
| 5 | 8.5 | 10.5 |
| 6 | 21.9 | 9.5 |

### 2E: Formation of Polymorphic Form X of Compound (1)

Polymorphic Form X of Compound (1) can be prepared by following the steps described below:

50 mg of EtOAc Solvate G was placed into an open 20 mL vial in a vacuum oven at 60° C. for 24 hours. After 24 hours the vial was removed and the powder was analyzed by XRPD. Form X was isostructural with EtOAc Solvate G so the location of the peaks listed in the xrpd patterns were within 0.2 degrees 2-theta of each other.

Characteristics of Form X of Compound (1): Certain representative XRPD peaks of Form X of Compound (1) are summarized in Table 4B below.

TABLE 4B
Certain representative XRPD Peaks of Form X

| | Form P |
|---|---|
| XRPD Peaks | Angle (2-Theta ± 0.2) |
| 1 | 7.5 |
| 2 | 12.1 |
| 3 | 13.0 |
| 4 | 13.8 |
| 5 | 16.2 |
| 6 | 19.7 |

### 2F: Formation of Polymorphic Form ZA of Compound (1)

Polymorphic Form ZA of Compound (1) can be prepared by following the steps described below:

3 mg of n-BuOAc solvate A of Compound (1) was placed into an aluminum DSC pan. The sample was heated at a rate of 10° C. per minute to 145° C. to remove n-BuOAc from n-BuOAc solvate A.

Characteristics of Form ZA of Compound (1): Certain representative XRPD peaks of Form ZA of Compound (1) are summarized in Table 4C below.

TABLE 4C
Certain representative XRPD Peaks of Form ZA

| | Form ZA |
|---|---|
| XRPD Peaks | Angle (2-Theta ± 0.2) |
| 1 | 5.2 |
| 2 | 10.2 |
| 3 | 16.5 |
| 4 | 18.6 |
| 5 | 19.8 |
| 6 | 20.3 |

### Example 3

### Formation of Co-Crystals of Compound (1)

### 3A: Formation of Urea Co-Crystal

Method A

Urea co-crystals of Compound (1) can be prepared by following the steps described below:

10 mg of Compound (1) was charged to a reactor. 1.35 mg of urea (1:1 molar ratio) was then charged to the reactor. Into the reactor was added dichloromethane (0.5 mL). The reaction mixture was stirred at room temperature for 8 days to form urea co-crystals of Compound (1). The resulting solids of urea co-crystals of Compound (1) were filtered and dried.

Method B

Alternatively, urea co-crystals of Compound (1) can be prepared by following the steps described below: 75 mg of Compound (1) was charged to a reactor. 10.13 mg of urea (1:1 molar ratio) was then charged to the reactor. Into the reactor was added acetonitrile (20 mL). The reaction mixture was stirred at room temperature for a day to form urea co-crystals of Compound (1). The resulting solids of urea co-crystals of Compound (1) were filtered and dried.

Certain representative XRPD peaks and DSC endotherm (° C.) of urea co-crystals of Compound (I) are summarized in Table 5 below.

TABLE 5
Certain representative XRPD peaks and DSC endotherm (° C.)
of urea co-crystals of Compound (1)

| | Urea Cocrystal | |
|---|---|---|
| DSC Endotherm (° C.) | 190° C. | |
| XRPD Peaks | Angle (2-Theta ± 0.2) | Intensity % |
| 1 | 18.4 | 100.0 |
| 2 | 12.1 | 69.1 |
| 3 | 15.6 | 65.0 |
| 4 | 20.1 | 52.6 |
| 5 | 10.8 | 46.5 |
| 6 | 11.7 | 44.1 |

### 3B: Formation of Nicotinamide Co-Crystal

Nicotinamide co-crystals of Compound (1) can be prepared by following the steps described below:

75 mg of Compound (1) was charged to a reactor. 16.13 mg of nicotinamide (1:1 molar ratio) was then charged to the reactor. Into the reactor was added acetonitrile (20 mL). The reaction mixture was stirred at room temperature for a day to form urea co-crystals of Compound (1). The resulting solids of urea co-crystals of Compound (1) were filtered and dried.

Certain representative XRPD peaks of nicotinamide co-crystals of Compound (1) are summarized in Table 6 below.

TABLE 6
Certain representative XRPD peaks of nicotinamide
co-crystals of Compound (1)

| | Nicotinamide Cocrystal | |
|---|---|---|
| XRPD Peaks | Angle (2-Theta ± 0.2) | Intensity % |
| 1 | 21.7 | 100.0 |
| 2 | 10.2 | 54.8 |
| 3 | 18.9 | 53.2 |
| 4 | 17.8 | 50.4 |
| 5 | 22.9 | 44.6 |
| 6 | 15.5 | 42.5 |

### 3C: Formation of Isonicotinamide Co-Crystal

Isonicotinamide co-crystals of Compound (1) can be prepared by following the steps described below:

75 mg of Compound (1) was charged to a reactor. 16.13 mg of isonicotinamide (1:1 molar ratio) was then charged to the reactor. Into the reactor was added acetonitrile (20 mL). The reaction mixture was stirred at room temperature for a day to form urea co-crystals of Compound (1). The resulting solids of urea co-crystals of Compound (1) were filtered and dried.

Certain representative XRPD peaks of isonicotinamide co-crystals of Compound (1) are summarized in Table 7 below.

TABLE 7
Certain representative XRPD peaks of isonicotinamide
co-crystals of Compound (1)

| | Isonicotinamide | |
|---|---|---|
| | Cocrystal | |
| XRPD Peaks | Ange (2-Theta ± 0.2) | Intensity % |
| 1 | 21.7 | 100.0 |
| 2 | 10.2 | 63.6 |
| 3 | 17.8 | 32.8 |
| 4 | 22.9 | 28.9 |
| 5 | 18.9 | 27.8 |
| 6 | 11.6 | 23.8 |

### Example 4

### Syntheses of Prodrugs of Compound 1

As used herein the term RT (min) refers to the LCMS retention time, in minutes, associated with the compound. NMR and Mass Spectroscopy data of certain specific compounds are summarized in Table 8.

### Preparation of Compound 2

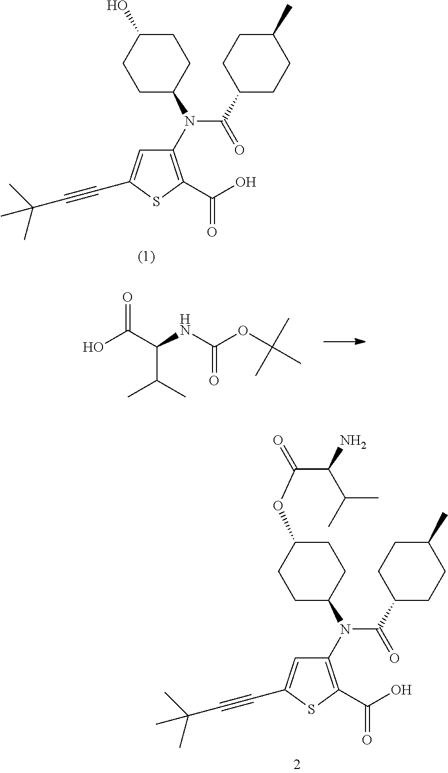

5-(3,3-Dimethylbut-1-ynyl)-3-[(trans-4-hydroxycyclohexyl)-(4-trans methylcyclohexanecarbonyl)amino]thiophene-2-carboxylic acid (compound (1), 300 mg, 0.67 mmol) was dissolved in dichloromethane (DCM, 15 mL). To this was added (2S)-2-(tert-butoxycarbonylamino)-3-methyl-butanoic acid Boc-L-valine (176 mg, 0.81 mmol), N,N-dimethylpyridin-4-amine (DMAP, 8.22 mg, 0.067 mmol), triethylamine (Et₃N, 136 mg, 187 μL, 1.35 mmol), and 3-(ethyliminomethyleneamino)-N,N-dimethyl-propan-1-amine hydrochloride (EDC, 129 mg, 0.67 mmol). The reaction was stirred overnight. The reaction mixture was then concentrated, diluted with ethyl acetate (EtOAc), washed with water, and the combined organic layers washed with brine and dried with sodium sulfate. Filtration and concentration gave a yellow oil, which was purified by column chromatography. The resulting product was then treated with 4N HCl in dioxane (15 mL) to give the desired compound 2 as the HCl salt (100 mg, 26%): MS: m/z (obs.): 545.4 [M+H]⁺; Retention time: 3.45 min; 1H NMR (300 MHz, MeOH) δ 7.04 (s, 1H), 4.75-4.58 (m, 1H), 4.39 (dt, J=14.5, 9.4 Hz, 1H), 3.85 (d, J=4.4 Hz, 1H), 3.80-3.68 (m, 1H), 3.61-3.51 (m, 1H), 2.24 (dt, J=14.0, 6.9 Hz, 1H), 2.01 (dd, J=15.2, 7.3 Hz, 6H), 1.60 (dd, J=28.5, 14.8 Hz, 9H), 1.34 (s, 9H), 1.18-0.99 (m, 3H), 0.81 (d, J=6.5 Hz, 3H), 0.66 (dd, J=25.3, 12.9 Hz, 1H).

### Preparation of Compound 3

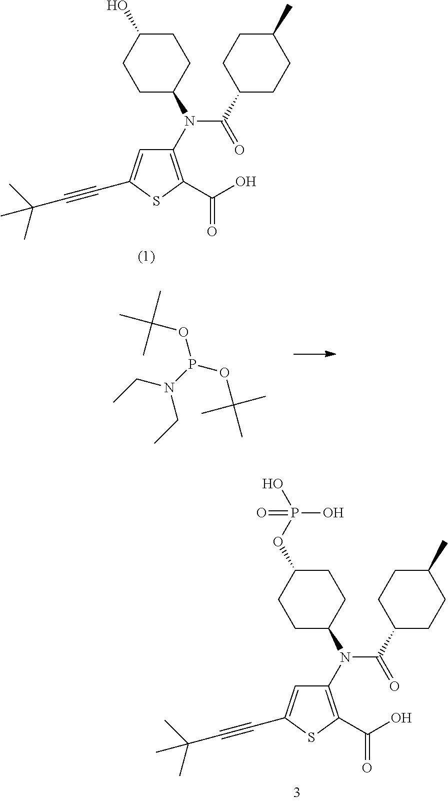

5-(3,3-Dimethylbut-1-ynyl)-3-[(trans 4-hydroxycyclohexyl)-(trans 4-methylcyclohexanecarbonyl)amino]thiophene-2-carboxylic acid (compound (1), 100 mg, 0.12 mmol) was dissolved in dichloromethane (DCM, 10.0 mL) and cooled to 0° C. Tetrazole (4.0 mg, 0.058 mmol) was added followed by N-(di-tert-butoxyphosphanyl)-N-ethyl-ethanamine (288 mg, 322 μL, 1.16 mmol). The reaction was stirred overnight at room temperature, then cooled to −78° C. 3-Chlorobenzenecarboperoxoic acid (MCPBA) (99.7 mg, 0.58 mmol) was added and the reaction stirred for 2 hours then quenched with aq. Na₂SO₃. The mixture was extracted with ethyl acetate and the extracts washed with water. The organic layer was concentrated to give a colorless oil, which was purified by ISCO silica gel chromatography and taken directly to the next step. To the product was added CH₂Cl₂ (5 mL) and 2,2,2-trifluoroacetic acid (TFA) (5 mL). The reaction was stirred for 2 hours, then concentrated and the product 3 purified by HPLC: MS: m/z (obs.): 526.39 [M+H]⁺; Retention time: 6.51 min; ¹H NMR (300 MHz, d6-DMSO) δ 7.18 (s, 1H), 4.29 (t, J=11.8 Hz, 1H), 3.83 (s, 1H), 2.53 (d, J=8.2 Hz, 3H), 1.84 (s, 2H), 1.75-1.33 (m, 7H), 1.30 (s, 9H), 1.27-1.09 (m, 3H), 0.90 (d, J=12.9 Hz, 2H), 0.76 (d, J=6.5 Hz, 2H), 0.70-0.47 (m, 2H); ³¹P NMR (121.5 MHz, d6-DMSO) δ −2.01 (s).

### Preparation of Compound 4

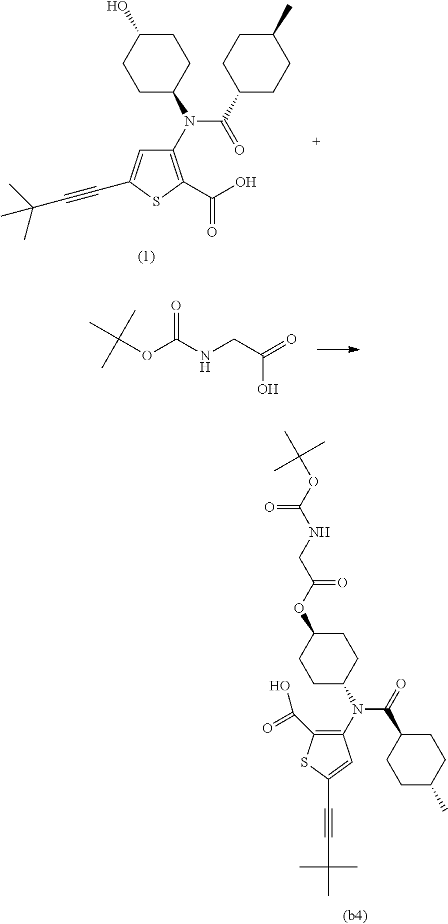

To a solution of 5-(3,3-dimethylbut-1-ynyl)-3-[(4-trans-hydroxycyclohexyl)-(4-trans-methylcyclohexanecarbonyl)amino]thiophene-2-carboxylic acid (compound (1), 75 mg, 0.17 mmol) and N-Boc-glycine (44.2 mg, 0.25 mmol) in CH₂Cl₂ (15 mL) was added 3-(ethyliminomethyleneamino)-N,N-dimethyl-propan-1-amine hydrochloride (EDC) (32.2 mg, 0.17 mmol), N,N-dimethylpyridin-4-amine (DMAP) (10.3 mg, 0.084 mmol) and Et₃N (34 mg, 0.33 mmol). The reaction mixture was stirred at ambient temperature overnight then the reaction mixture was evaporated and purified by ISCO silica gel chromatography to give compound (b4), [O—(N-t-Butoxycarbonyl)-glycyl]-5-(3,3-dimethylbut-1-ynyl)-3-[(4-trans-hydroxycyclohexyl)-(4-trans-methylcyclohexanecarbonyl)amino]thiophene-2-carboxylic acid: MS: m/z (obs.): 603.17 [M+H]⁺; Retention time: 2.31 min.

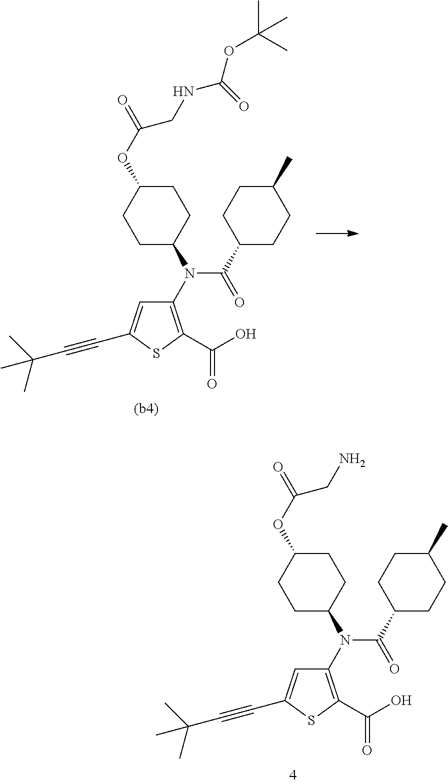

[O—(N-t-Butoxycarbonyl)-glycyl]-5-(3,3-dimethylbut-1-ynyl)-3-[(4-trans-hydroxycyclohexyl)-(4-trans-methylcyclohexanecarbonyl)amino]thiophene-2-carboxylic acid (Compound (b4), 40 mg, 0.066 mmol) was treated with 4N HCl in dioxane (1 mL) and stirred at RT overnight. Then the reaction mixture was concentrated and purified by HPLC to give compound 4 (11 mg): MS: m/z (obs.): 503.35 [M+H]⁺; Retention time: 2.24 min.

### Preparation of Compound 5

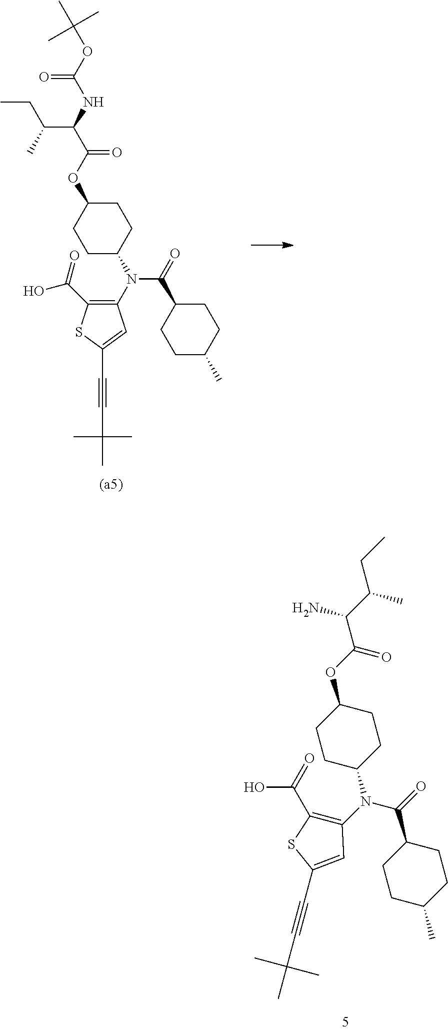 [SMILES: CC[C@@H](C)[C@@H](NC(=O)OC(C)(C)C)C(=O)O[C@H]1CC[C@H](N(c2cc(C#CC(C)(C)C)sc2C(=O)O)C(=O)[C@H]2CC[C@H](C)CC2)CC1 ; CC[C@H](C)[C@@H](N)C(=O)O[C@H]1CC[C@H](N(c2cc(C#CC(C)(C)C)sc2C(=O)O)C(=O)[C@H]2CC[C@H](C)CC2)CC1]

Compound (a5), [O—(N-t-Butoxycarbonyl)-D-isoleucyl]-5-(3,3-dimethylbut-1-ynyl)-3-[(4-trans-hydroxycyclohexyl)-(4-trans-methylcyclohexanecarbonyl)amino]thiophene-2-carboxylic acid (prepared from Boc-D-isoleucine as described for Compounds 1 & 4 above) was treated with 4N HCl in dioxane (10 mL) and stirred at RT overnight. Then the reaction mixture was concentrated and purified by HPLC to give compound 5: MS: m/z (obs.): 559.4 [M+H]⁺; Retention time: 2.39 min.

### Preparation of Compound 6

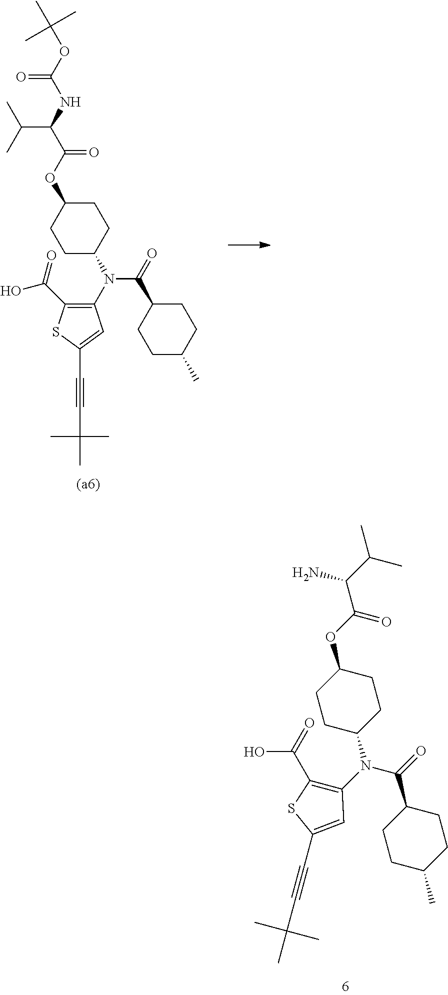 [SMILES: CC(C)[C@@H](NC(=O)OC(C)(C)C)C(=O)O[C@H]1CC[C@H](N(c2cc(C#CC(C)(C)C)sc2C(=O)O)C(=O)[C@H]2CC[C@H](C)CC2)CC1 ; CC(C)[C@@H](N)C(=O)O[C@H]1CC[C@H](N(c2cc(C#CC(C)(C)C)sc2C(=O)O)C(=O)[C@H]2CC[C@H](C)CC2)CC1]

Compound (a6), [O—(N-t-Butoxycarbonyl)-D-valinyl]-5-(3,3-dimethylbut-1-ynyl)-3-[(4-trans-hydroxycyclohexyl)-(4-trans-methylcyclohexanecarbonyl)amino]thiophene-2-carboxylic acid (30 mg) (prepared from Boc-D-valine as described for Compounds 1 & 4 above) was treated with 4N HCl in dioxane (10 mL) and stirred at RT overnight. Then the reaction mixture was concentrated and purified by HPLC to give compound 6: MS: m/z (obs.): 545.39 [M+H]⁺; Retention time: 2.35 min.

### Preparation of Compound 7

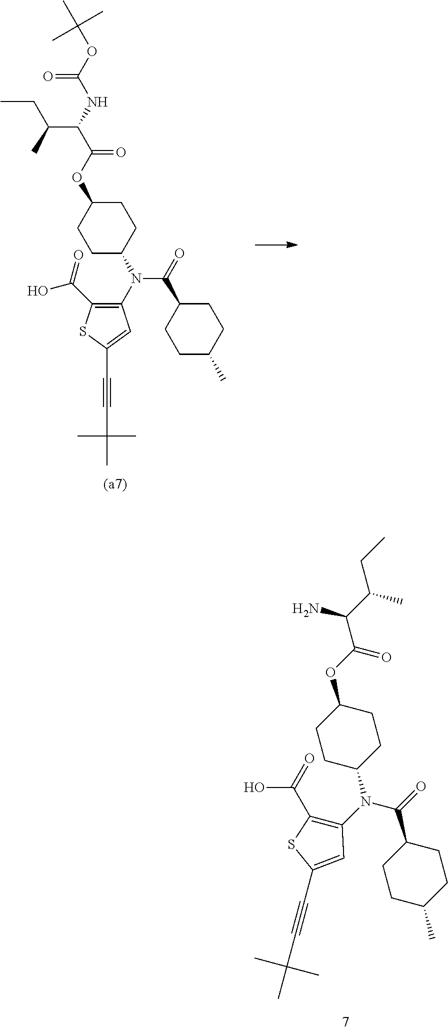 [SMILES: CC[C@H](C)[C@H](NC(=O)OC(C)(C)C)C(=O)O[C@H]1CC[C@H](N(c2cc(C#CC(C)(C)C)sc2C(=O)O)C(=O)[C@H]2CC[C@H](C)CC2)CC1 ; CC[C@H](C)[C@H](N)C(=O)O[C@H]1CC[C@H](N(c2cc(C#CC(C)(C)C)sc2C(=O)O)C(=O)[C@H]2CC[C@H](C)CC2)CC1]

Compound (a7), (O—(N-t-Butoxycarbonyl)-L-isoleucyl)-5-(3,3-dimethylbut-1-ynyl)-3-[(4-trans-hydroxycyclohexyl)-(4-trans-methylcyclohexanecarbonyl)amino]thiophene-2-carboxylic acid (prepared from Boc-L-isoleucine as described for Compounds 2 & 4 above) (35 mg) was treated with 4N HCl in dioxane (10 mL) and stirred at RT for overnight. Then the reaction mixture was concentrated and purified by HPLC to give compound 7: MS: m/z (obs.): 559.47 [M+H]⁺; Retention time: 3.2 min.

### Preparation of Compound 8

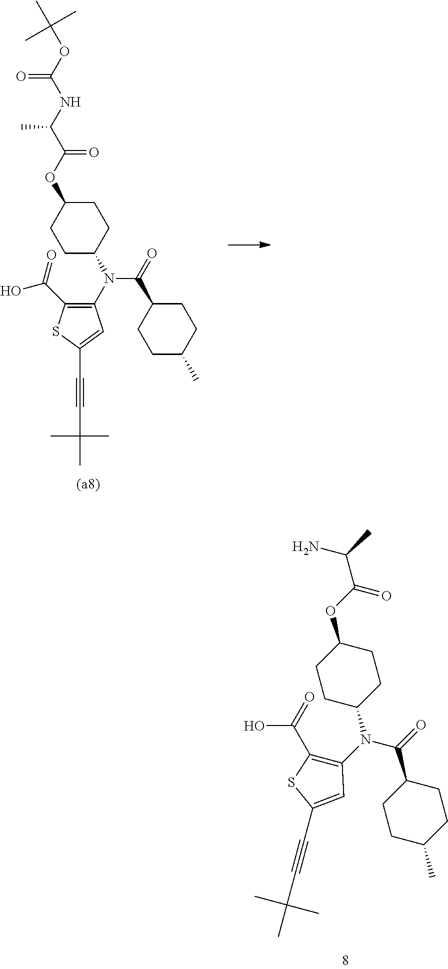 [SMILES: C[C@H](NC(=O)OC(C)(C)C)C(=O)O[C@H]1CC[C@H](N(c2cc(C#CC(C)(C)C)sc2C(=O)O)C(=O)[C@H]2CC[C@H](C)CC2)CC1 ; C[C@H](N)C(=O)O[C@H]1CC[C@H](N(c2cc(C#CC(C)(C)C)sc2C(=O)O)C(=O)[C@H]2CC[C@H](C)CC2)CC1]

Compound (a8), (O—(N-t-Butoxycarbonyl)-L-alanyl)-5-(3,3-dimethylbut-1-ynyl)-3-[(4-trans-hydroxycyclohexyl)-(4-trans-methylcyclohexanecarbonyl)amino]thiophene-2-carboxylic acid (prepared from Boc-L-alanine as described for Compounds 2 & 4 above) (25 mg) was taken in 4N HCl in dioxane and stirred at RT overnight. Then the reaction mixture was concentrated and purified by HPLC to give compound 8: MS: m/z (obs.): 517.43 [M+H]⁺; Retention time: 2.99 min.

### Preparation of Compound 9

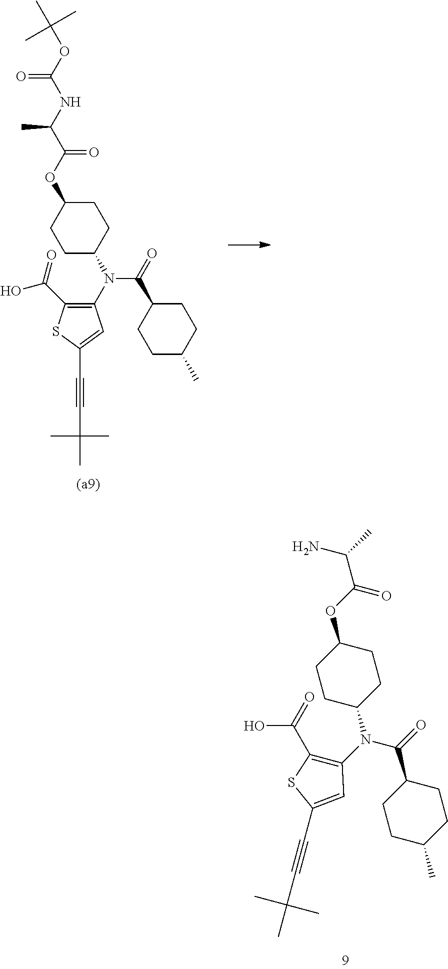 [SMILES: C[C@@H](NC(=O)OC(C)(C)C)C(=O)O[C@H]1CC[C@H](N(c2cc(C#CC(C)(C)C)sc2C(=O)O)C(=O)[C@H]2CC[C@H](C)CC2)CC1 ; C[C@@H](N)C(=O)O[C@H]1CC[C@H](N(c2cc(C#CC(C)(C)C)sc2C(=O)O)C(=O)[C@H]2CC[C@H](C)CC2)CC1]

Compound (a9), (O—(N-t-Butoxycarbonyl)-D-alanyl)-5-(3,3-dimethylbut-1-ynyl)-3-[(4-trans-hydroxycyclohexyl)-(4-trans-methylcyclohexanecarbonyl)amino]thiophene-2-carboxylic acid (prepared from Boc-D-alanine as described for Compounds 2 & 4 above) (35 mg, 0.058 mmol) was treated with 4N HCl in dioxane (10 mL) and stirred at RT overnight. Then the reaction mixture was concentrated and purified by HPLC to give compound 9: MS: m/z (obs.): 517.43 [M+H]⁺; Retention time: 3.0 min.

TABLE 8
LCMS and NMR data of Compounds 2-9 (Prodrugs of Compound 1)

| | LCMS | LCMS | |
|---|---|---|---|
| Compounds | [M + H]⁺ | RT | NMR |
| 2 | 545.45 | 3.45 | 1H NMR (300 MHz, MeOH) δ 7.04 (s, 1H), 4.75 − 4.58 (m, 1H), |
| | | | 4.39 (dt, J = 14.5, 9.4 Hz, 1H), 3.85 (d, J = 4.4 Hz, 1H), 3.80 − 3.68 |
| | | | (m, 1H), 3.61 − 3.51 (m, 1H), 2.24 (dt, J = 14.0, 6.9 Hz, 1H), 2.01 |
| | | | (dd, J = 15.2, 7.3 Hz, 6H), 1.60 (dd, J = 28.5, 14.8 Hz, 9H), |
| | | | 1.34 (s, 9H), 1.18 − 0.99 (m, 3H), 0.81 (d, J = 6.5 Hz, 3H), 0.66 |
| | | | (dd, J = 25.3, 12.9 Hz, 1H). |
| 3 | 526.39 | 6.51 | ¹H NMR (300 MHz, d6-DMSO) δ 7.18 (s, 1H), 4.29 (t, J = 11.8 Hz, |
| | | | 1H), 3.83 (s, 1H), 2.53 (d, J = 8.2 Hz, 3H), 1.84 (s, 2H), 1.75 − 1.33 |
| | | | (m, 7H), 1.30 (s, 9H), 1.27 − 1.09 (m, 3H), 0.90 (d, J = 12.9 Hz, 2H), |
| | | | 0.76 (d, J = 6.5 Hz, 2H), 0.70 − 0.47 (m, 2H); ³¹P NMR (121.5 MHz, |
| | | | d6-DMSO) δ −2.01 (s). |
| 4 | 503.35 | 2.24 | |
| 5 | 559.4 | 2.39 | |
| 6 | 545.39 | 2.35 | |
| 7 | 559.47 | 3.2 | |
| 8 | 517.43 | 2.99 | |
| 9 | 517.43 | 3 | |

### Example 5

### Formation of Solvates of Compound (1)

### DSC Measurements

DSC was conducted on a TA Instruments model Q2000 V24.3 calorimeter (Asset Tag V014080). Approximately 1-2 mg of solid sample was placed in an aluminum hermetic DSC pan with a crimped lid with a pinhole. The sample cell was heated under nitrogen purge at 10° C. per minute to 300° C.

Bruker D8 Discover XRPD Experimental Details.

The XRPD patterns were acquired at room temperature in reflection mode using a Bruker D8 Discover diffractometer (Asset Tag V012842) equipped with a sealed tube source and a Hi-Star area detector (Bruker AXS, Madison, Wis.). The X-Ray generator was operating at a voltage of 40 kV and a current of 35 mA. The powder sample was placed in an aluminum holder. Two frames were registered with an exposure time of 120 s each. The data were subsequently integrated over the range of 4°-40° 2θ with a step size of 0.02° and merged into one continuous pattern.

### 5A: Formation of Hydrate A (Compound (1).1H₂O)

Hydrate A of Compound (1) can be prepared by following the steps described below:

121 mg of Compound (1) was charged to a vial. 2 mL of DI water was then charged to the vial. The mixture was stirred at room temperature for 2 days to form Compound (1).1 H₂O and the resulting solids were filtered and dried. TGA data indicated a hydrate solvate with a stoichiometry of approximately 1:1 (Compound (1):H₂O).

Characteristics of Hydrate A of Compound (1): Certain representative XRPD peaks of hydrate A of Compound (1) are summarized in Table 9 below.

TABLE 9
Certain representative XRPD Peaks of Hydrate A of Compound (1)

| | Hydrate A | |
|---|---|---|
| XRPD Peaks | Angle (2-Theta ± 0.2) | Intensity % |
| 1 | 20.3 | 100.0 |
| 2 | 9.1 | 41.1 |
| 3 | 18.4 | 31.5 |
| 4 | 7.6 | 27.3 |
| 5 | 19.6 | 25.7 |
| 6 | 14.6 | 24.4 |

### 5B: Formation of Hydrate B (Compound (1).2H₂O)

Hydrate B of Compound (1) can be prepared by following the steps described below:

20 mg of Compound (1) was charged to a vial. 0.5 mL of DI water was then charged to the vial. The mixture was stirred at room temperature for 3 weeks to form Compound (1).2 H₂O and the resulting solids were filtered and dried. TGA data indicated a hydrate solvate with a stoichiometry of approximately 1:2 (Compound (1):H₂O).

Characteristics of Hydrate B of Compound (1): Certain representative XRPD peaks and DSC endotherm (° C.) of hydrate B of Compound (1) are summarized in Table 10 below.

TABLE 10
Certain representative XRPD Peaks and DSC
Endotherm of Hydrate B of Compound (1)

| | Hydrate B | |
|---|---|---|
| DSC Endotherm (° C.) | 60° C. | |
| XRPD Peaks | Angle (2-Theta ± 0.2) | Intensity % |
| 1 | 19.0 | 100.0 |
| 2 | 9.9 | 84.2 |
| 3 | 12.9 | 36.5 |
| 4 | 11.6 | 31.9 |
| 5 | 15.9 | 27.6 |
| 6 | 24.2 | 20.3 |

### 5C: Formation of Methanol Solvates of Compound (1) (Compound (1).MeOH)

Methanol solvates of Compound (1) can be prepared by following the steps described below:

A slurry 20 mg of Compound (1) in 500 microliters of MeOH was stirred at room temperature for 3 weeks in a capped HPLC vial to form Compound (1).MeOH. The solids were collected by filtration and analyzed by XRPD. TGA data indicated a methanol solvate with a stoichiometry of approximately 1:1 (Compound (1):methanol).

Characteristics of methanol solvates of Compound (1): Certain representative XRPD peaks of methanol solvates of Compound (1) are summarized in Table 11 below.

TABLE 11
Certain representative XRPD of Methanol Solvates of Compound (1)

| | Methanol Solvates | |
|---|---|---|
| XRPD Peaks | Angle (2-Theta ± 0.2) | Intensity % |
| 1 | 8.0 | 100 |
| 2 | 10.3 | 68 |
| 3 | 17.9 | 59 |
| 4 | 19.9 | 63 |
| 5 | 20.6 | 39 |
| 6 | 22.1 | 45 |

### 5D: Formation of Ethanol/Isopropanol Solvates of Compound (1) (Compound (1).EtOH.IPA)

Ethanol/Isopropanol solvates of Compound (1) (94.7 vol % EtOH/5.3 vol % IPA) can be prepared by following the steps described below:

A slurry containing 100 mg of Compound (1) in EtOH/IPA (95.7% EtOH/4.7% IPA) in a 2 mL vial was stirred at room temperature overnight to form Compound (1).EtOH.IPA. The solvent was decanted off, giving the remaining wet-cake which was analyzed by XRPD.

Characteristics of EtOH/IPA solvates of Compound (1): Certain representative XRPD peaks of EtOH/IPA solvates of Compound (1) are summarized in Table 12 below.

TABLE 12
Certain representative XRPD of EtOH/IPA Solvates of Compound (1)

| | EtOH/IPA Solvates | |
|---|---|---|
| XRPD Peaks | Angle (2-Theta ± 0.2) | Intensity % |
| 1 | 8.7 | 18 |
| 2 | 9.3 | 77 |
| 3 | 18.3 | 100 |
| 4 | 19.8 | 24 |
| 5 | 15.5 | 45 |
| 6 | 23.1 | 18 |

### 5E: Formation of Acetone Solvates of Compound (1) (Compound (1).acetone)

Acetone solvates of Compound (1) (Compound (1).1 acetone) can be prepared by following the steps described below:

Crystals of acetone solvate of Compound (1) (1:1 stoichiometry) were grown by slow evaporation from a solution of Compound (1) in acetone. The crystals were collected and analyzed by XRPD. TGA data indicated an acetone solvate with a stoichiometry of approximately 1:1 (Compound (1):acetone).

Characteristics of acetone solvates of Compound (1): Certain representative XRPD peaks of acetone solvates of Compound (1) are summarized in Table 13 below.

TABLE 13
Certain representative XRPD of Acetone Solvates of Compound (1)

| | Acetone Solvates | |
|---|---|---|
| XRPD Peaks | Angle (2-Theta ± 0.2) | Intensity % |
| 1 | 7.7 | 100 |
| 2 | 10.4 | 63 |
| 3 | 11.3 | 43 |
| 4 | 16.5 | 69 |
| 5 | 19.1 | 37 |
| 6 | 21.1 | 83 |

### 5F: Formation of Ethylacetate Solvates of Compound (1) (Compound (1).EtOAc)

Forms A-F of ethyl acetate solvates of Compound (1) (Compound (1).EtOAc) can be prepared by following the steps described below:

### 1. Form A:

A slurry containing 100 mg of Compound (1) in EtOAc in a 2 mL vial was stirred at room temperature overnight. The solvent was decanted off giving the remaining wet-cake which was analyzed by XRPD. TGA data indicated an EtOAc solvate with a stoichiometry of approximately 3:1 (Compound (1):EtOAc).

Characteristics of ethyl acetate Form A solvate of Compound (1): Certain representative XRPD peaks of ethyl acetate Form A solvate of Compound (1) are summarized in Table 14 below.

TABLE 14
Certain representative XRPD of EtOAc solvate A

| | EtOAc solvate A | |
|---|---|---|
| XRPD Peaks | Angle (2-Theta ± 0.2) | Intensity % |
| 1 | 6.4 | 96 |
| 2 | 7.0 | 100 |
| 3 | 9.7 | 62 |
| 4 | 16.1 | 49 |
| 5 | 18.7 | 59 |
| 6 | 22.6 | 47 |

### 2. Form B:

A slurry containing 20 mg of Compound (1) in 500 microliters of EtOAc in a capped vial was stirred at room temperature for 3 weeks. The solids were collected by filtration and analyzed by XRPD.

Characteristics of ethyl acetate Form B solvate of Compound (1): Certain representative XRPD peaks of ethyl acetate Form B solvate of Compound (1) are summarized in Table 15 below.

TABLE 15
Certain representative XRPD of EtOAc solvate B

| | EtOAc solvate B |
|---|---|
| XRPD Peaks | Angle (2-Theta ± 0.2) |
| 1 | 6.4 |
| 2 | 7.3 |
| 3 | 8.1 |
| 4 | 9.0 |
| 5 | 18.1 |
| 6 | 19.7 |

### 3. Form C:

Approximately 20 kg of Compound (1) was added to a reactor. 200 kg of 2-MeTHF was then charged to the reactor. 200 kg of EtOAc was then added to the reactor and the solution was rotovapped at 100 mmHg and 30° C. which resulted in an oil being obtained. The reactor was then charged with 591 kg of EtOAc which was then rotovapped at 50 mmHg and 30° C. The solid residue was submitted for XRPD.

Characteristics of ethyl acetate Form C solvate of Compound (1): Certain representative XRPD peaks of ethyl acetate Form C solvate of Compound (1) are summarized in Table 16 below.

TABLE 16
Certain representative XRPD of EtOAc solvate C

| | EtOAc solvate C | |
|---|---|---|
| XRPD Peaks | Angle (2-Theta ± 0.2) | Intensity % |
| 1 | 6.3 | 100 |
| 2 | 14.6 | 17 |
| 3 | 15.6 | 13 |
| 4 | 18.9 | 34 |
| 5 | 20.4 | 15 |
| 6 | 22.2 | 25 |

### 4. Form D:

550 mg of Compound (1) was added to 2 mL of EtOAc. The slurry was shaken for 4 days at 400 rpm between 20° C. and 25° C. The sample was then filtered and analyzed for XRPD.

Characteristics of ethyl acetate Form D solvate of Compound (1): Certain representative XRPD peaks of ethyl acetate Form D solvate of Compound (1) are summarized in Table 17 below.

TABLE 17
Certain representative XRPD of EtOAc solvate D

| | EtOAc solvate D | |
|---|---|---|
| XRPD Peaks | Angle (2-Theta ± 0.2) | Intensity % |
| 1 | 4.9 | 43 |
| 2 | 9.8 | 71 |
| 3 | 14.6 | 48 |
| 4 | 16.6 | 100 |
| 5 | 20.2 | 91 |
| 6 | 21.2 | 75 |

### 5. Form E:

60 mg of Compound (1) was added to 1 mL of EtOAc. The suspension was cooled to 10° C. and stirred for 4 days. The sample was then filtered and analyzed for XRPD.

Characteristics of ethyl acetate Form E solvate of Compound (1): Certain representative XRPD peaks of ethyl acetate Form E solvate of Compound (1) are summarized in Table 18 below.

TABLE 18
Certain representative XRPD of EtOAc solvate E

| | EtOAc solvate E | |
|---|---|---|
| XRPD Peaks | Angle (2-Theta ± 0.2) | Intensity % |
| 1 | 9.7 | 57 |
| 2 | 11.6 | 35 |
| 3 | 12.4 | 62 |
| 4 | 15.7 | 36 |
| 5 | 18.8 | 100 |
| 6 | 23.9 | 29 |

### 6. Form F:

Compound (1) (30.46 g, 66.27 mmol) was charged o a 500 ml round bottom flask. Charged 2-Me-THF (182.8 mL) and started agitation. Sodium hydroxide (122.6 mL of 2 M, 245.2 mmol) was then charged to the solution. The reaction mixture was heated to 68° C. and stirred overnight at 70° C. The reaction was cooled to 0° C. Citric acid (157.0 mL of 30% w/v, 245.2 mmol) was added. The resulting mixture was stirred for 30 minutes. Phases were separated and water (152.3 mL) was added to the organic layer. The phases were allowed to separate. The batch was distilled down to 3 volume. 2-MeTHF (91.38 mL) was added and the batch was distilled down to 3 vol. The batch was distilled down to 3 volume. 2-MeTHF (91.38 mL) was added and the batch was distilled down to 3 vol EtOAc (304.6 mL) was charged and the batch was distilled down to 2-3 volumes. The batch was adjusted to 10 volumes by adding 7-8 volumes of EtOAc. The batch was distilled down to 2-3 volumes. The batch was adjusted to 10 volumes by adding 7-8 volumes of EtOAc. The batch was distilled down to 2-3 volumes. The batch was adjusted to 10 volumes by adding 7-8 volumes of EtOAc. Adjusted batch volume to 10 volume total and stir heat batch to 50° C. A small sample was taken and filtered after the temperature of 50° was reached.

TGA data indicated an EtOAc solvate with a stoichiometry of approximately 2:1 (Compound (I):EtOAc).

Characteristics of ethyl acetate Form F solvate of Compound (1): Certain representative XRPD peaks of ethyl acetate Form F solvate of Compound (1) are summarized in Table 19 below.

TABLE 19
Certain representative XRPD of EtOAc solvate F

| | EtOAc solvate F |
|---|---|
| XRPD Peaks | Angle (2-Theta ± 0.2) |
| 1 | 7.2 |
| 2 | 9.7 |
| 3 | 17.1 |
| 4 | 18.8 |
| 5 | 22.7 |
| 6 | 23.5 |

### 7. Form G:

1 g of Compound (1) was added to 5 mL of EtOAc. The suspension was stirred at room temperature for 1 day. Alternatively, 100 mg of ethylacetate solvate seeds were added into the suspension of Compound (1) in EtOAc and the resulting mixture was stirred at room temperature for a day. The sample was then filtered and analyzed for XRPD. TGA data indicated an EtOAc solvate with a stoichiometry of approximately 1:1 (Compound (1):EtOAc).

Certain representative XRPD peaks of EtOAc solvate G are summarized in Table 12 below.

TABLE 12
Certain representative XRPD of EtOAc solvate G

| | EtOAc solvate G |
|---|---|
| XRPD Peaks | Angle (2-Theta ± 0.2) |
| 1 | 7.5 |
| 2 | 12.1 |
| 3 | 13.0 |
| 4 | 13.7 |
| 5 | 16.2 |
| 6 | 19.7 |

### 5G: Formation of Isopropyl Acetate Solvates of Compound (1) (Compound (1).IPac)

A slurry containing 100 mg of Compound (1) in isopropylacetate in a 2 mL vial was stirred at room temperature overnight. The solvent was decanted off giving the remaining wet-cake which was analyzed by XRPD.

Characteristics of isopropylacetate solvate of Compound (1): XRPD data of isopropylacetate solvate of Compound (1) showed that the ethyl acetate Form A solvate of Compound (1) and isopropylacetate solvate of Compound (1) were isostructural to each other, sharing the same representative XRPD peaks summarized in Table 20 below.

TABLE 20
Certain representative XRPD of isopropyl acetate solvate of Compound (1)

| | Isopropylacetate Solvate | |
|---|---|---|
| XRPD Peaks | Angle (2-Theta ± 0.2) | Intensity % |
| 1 | 6.4 | 96 |
| 2 | 7.0 | 100 |
| 3 | 9.7 | 62 |
| 4 | 16.1 | 49 |
| 5 | 18.7 | 59 |
| 6 | 22.6 | 47 |

### 5H: Formation of Ethylacetate/2-Methyl THF Solvates of Compound (1) (Compound (1).ethylacetate.2-methyl THF)

1. EtOAc/2-MethylTHF (70%/30% w/w)

A slurry 100 mg of Compound (1) in 1 mL of 70% EtOAc/30% 2-MethylTHF (w/w) at 5° C. was stirred for 24 hours in a capped vial. The solids were collected by filtration and analyzed by XRPD.

Characteristics of ethylacetate/2-methyl THF solvates of Compound (1): Certain representative XRPD peaks are summarized in Table 21 below.

TABLE 21
Certain representative XRPD of ethylacetate/2-methyl THF
solvate of Compound (1)

| | ethylacetate/2-methyl |
|---|---|
| | THF Solvate |
| XRPD Peaks | Angle (2-Theta ± 0.2) |
| 1 | 5.5 |
| 2 | 9.3 |
| 3 | 10.9 |
| 4 | 12.6 |
| 5 | 17.7 |
| 6 | 19.7 |

2. Form B: EtOAc/2-MethylTHF (90%/10% w/w)

A slurry 100 mg of Compound (1) in 1 mL of 90% EtOAc/10% 2-MethylTHF (w/w) at room temperature was stirred for 24 hours in a capped vial. The solids were collected by filtration and analyzed by XRPD.

Characteristics of ethyl acetate/2-methyl THF solvates of Compound (1): Certain representative XRPD peaks are summarized in Table 22 below.

TABLE 22
Certain representative XRPD of ethylacetate/2-methyl THF
solvate of Compound (1)

| | ethylacetate/2-methyl |
|---|---|
| | THF Solvate |
| XRPD Peaks | Angle (2-Theta ± 0.2) |
| 1 | 6.5 |
| 2 | 7.3 |
| 3 | 9.1 |
| 4 | 17.0 |
| 5 | 18.7 |
| 6 | 23.5 |

### 5I: Formation of Ethanol Solvates of Compound (1) (Compound (1).Ethanol)

Slurry of 100 mg of Compound (1) in 500 microliters of EtOH was stirred for 24 hours in a capped vial. The solids are collected by filtration and analyzed by XRPD. TGA data indicated an ethanol solvate with a stoichiometry of approximately 1:1 (Compound (1):EtOH).

Characteristics of ethanol solvates of Compound (1): Certain representative XRPD peaks are summarized in Table 23 below.

TABLE 23
Certain representative XRPD of ethanol solvates of Compound (1)

| | Ethanol Solvate |
|---|---|
| XRPD Peaks | Angle (2-Theta ± 0.2) |
| 1 | 8.1 |
| 2 | 10.1 |
| 3 | 18.1 |
| 4 | 19.6 |
| 5 | 21.7 |
| 6 | 25.6 |

### 5J: Formation of n-Butylacetate Solvates of Compound (1) (Compound (1).nBuOAc)

n-Butylacetate solvates A-C of Compound (1) (Compound (1).nBuOAc) can be prepared by following the steps described below:

1. n-Butylacetate Solvate A:

A mixture of 500 mg of Compound (1) in 5 mL of n-BuOAc was stirred for 3 days in a capped 20 dram vial. The solids were collected by filtration and analyzed. TGA data (not shown) indicated an n-BuOAc solvate with a stoichiometry of approximately 2:1 (Compound (1): n-BuOAc).

Characteristics of n-Butylacetate solvate A of Compound (1): Certain representative XRPD peaks of n-Butylacetate solvate A are summarized in Table 24 below.

TABLE 24
Certain representative XRPD of n-Butylacetate Solvate A

| | n-Butylacetate Solvate A |
|---|---|
| XRPD Peaks | Angle (2-Theta ± 0.2) |
| 1 | 9.7 |
| 2 | 14.9 |
| 3 | 16.5 |
| 4 | 19.6 |
| 5 | 20.0 |
| 6 | 21.0 |

2. n-Butylacetate Solvate B:

109 mg of Compound (1) was dissolved in 2 mL of n-BuOAc. Precipitation began to occur after a few minutes. The solvent was then evaporated under ambient conditions for 2 weeks. The resulting material was collected and characterized. TGA data (not shown) indicated an n-BuOAc solvate with a stoichiometry of approximately 1:1 (Compound (1): n-BuOAc).

Characteristics of n-Butylacetate solvate B of Compound (1): Certain representative XRPD peaks of n-Butylacetate solvate B are summarized in Table 25 below.

TABLE 25
Certain representative XRPD of n-Butylacetate Solvate B of Compound (1)

| | n-Butylacetate Solvate B |
|---|---|
| XRPD Peaks | Angle (2-Theta ± 0.2) |
| 1 | 6.4 |
| 2 | 6.9 |
| 3 | 17.5 |
| 4 | 18.2 |
| 5 | 18.9 |
| 6 | 23.2 |

3. n-Butylacetate Solvate C:

A mixture of Compound (1) and n-BuOAc was stirred at room temperature similarly as described above for n-Butylacetate solvates A and B. TGA data indicated an n-BuOAc solvate with a stoichiometry of approximately 4:1 (Compound (1): n-BuOAc).

Characteristics of n-Butylacetate solvate C of Compound (1): Certain representative XRPD peaks of n-Butylacetate Solvate C are summarized in Table 26 below.

TABLE 26
Certain representative XRPD of n-Butylacetate Solvate C of Compound (1)

| | n-Butylacetate Solvate C |
|---|---|
| XRPD Peaks | Angle (2-Theta ± 0.2) |
| 1 | 6.9 |
| 2 | 9.6 |
| 3 | 15.9 |
| 4 | 16.9 |
| 5 | 18.6 |
| 6 | 19.3 |

### 5K: Formation of Heptane Solvates of Compound (1) (Compound (1).Heptane)

Heptane solvates A-D of Compound (1) (Compound (1).Heptane can be prepared by following the steps described below:

### 1. Heptane Solvate A:

A mixture of Compound (1) in heptane was stirred at room temperature. The solids were collected by filtration and analyzed.

Characteristics of heptane solvate A of Compound (1): Certain representative XRPD peaks of heptane solvate A are summarized in Table 27 below.

TABLE 27
Certain representative XRPD of Heptane Solvate A

| | Heptane Solvate A |
|---|---|
| XRPD Peaks | Angle (2-Theta ± 0.2) |
| 1 | 10.7 |
| 2 | 12.3 |
| 3 | 14.0 |
| 4 | 17.2 |
| 5 | 19.6 |
| 6 | 21.3 |

### 2. Heptane Solvate B:

106 mg of amorphous Compound (1) was added to a solvent mixture of 0.5 mL EtOAc and 0.5 mL heptane. The suspension was agitated for 7 days at 20° C. The solids were isolated by centrifugation filtration and analyzed.

Characteristics of heptane solvate B of Compound (1): Certain representative XRPD peaks of heptane solvate B are summarized in Table 28 below.

TABLE 28
Certain representative XRPD of Heptane Solvate B

| | Heptane Solvate B |
|---|---|
| XRPD Peaks | Angle (2-Theta ± 0.2) |
| 1 | 5.5 |
| 2 | 7.5 |
| 3 | 9.3 |
| 4 | 10.9 |
| 5 | 16.5 |
| 6 | 22.1 |

### 3. Heptane Solvate C:

A slurry in 1 mL of heptane was made by addition of approximately 50 mg of Compound (1). The material was stirred for 60 days at 20° C. The material was then filtered and analyzed by XRPD.

Characteristics of heptane solvate C of Compound (1): Certain representative XRPD peaks of heptane solvate C are summarized in Table 29 below.

TABLE 29
Certain representative XRPD of Heptane Solvate C

| | Heptane Solvate C |
|---|---|
| XRPD Peaks | Angle (2-Theta ± 0.2) |
| 1 | 10.4 |
| 2 | 11.2 |
| 3 | 13.4 |
| 4 | 16.9 |
| 5 | 19.3 |
| 6 | 19.8 |

### 4. Heptane Solvate D:

A slurry in 1 mL of heptane was made by addition of approximately 50 mg of Compound (1). The material was stirred for 60 days at 25° C. The material was then filtered and analyzed by XRPD.

Characteristics of heptane solvate D of Compound (1): Certain representative XRPD peaks of heptane solvate D are summarized in Table 30 below.

TABLE 30
Certain representative XRPD of Heptane Solvate D

| | Heptane Solvate D |
|---|---|
| XRPD Peaks | Angle (2-Theta ± 0.2) |
| 1 | 10.3 |
| 2 | 13.7 |
| 3 | 16.9 |
| 4 | 18.9 |
| 5 | 19.3 |
| 6 | 20.4 |

### 5. Heptane Solvate E:

52.3 mg of Compound (1) was dispersed in 1 mL heptane. The suspension was stirred at room temperature for 5 days. The suspension was then filtered and analyzed by XRPD.

Characteristics of heptane solvate E of Compound (1): Certain representative XRPD peaks of heptane solvate E are summarized in Table 31 below.

TABLE 31
Certain representative XRPD of Heptane Solvate E

| | Heptane Solvate D |
|---|---|
| XRPD Peaks | Angle (2-Theta ± 0.2) |
| 1 | 5.0 |
| 2 | 10.2 |
| 3 | 16.9 |
| 4 | 19.3 |
| 5 | 20.5 |
| 6 | 21.5 |

### 3L: Formation of MEK Solvates of Compound (1) (Compound (1).MEK)

MEK solvates of Compound (1) (Compound (1).MEK can be prepared by following the steps described below:

400 mg of Compound (1) was added to 1 mL of MEK (methylethyl ketone (2-butanone)) in a vial. A thick slurry was obtained after vortexing the vial for 1 minute. The resulting mixture was then stirred for 4 hours. The solids from the wet slurry were analyzed by ¹³C SSNMR.

Characteristics of MEK solvates of Compound (1): Certain representative peaks of ¹³C SSNMR spectrum of MEK solvates are summarized in Table 32 below.

TABLE 32
Certain representative XRPD of Heptane Solvate A

| ¹³C SSNMR Shifts | ppm (± 0.2) |
|---|---|
| 1 | 205.7 |
| 2 | 132.5 |
| 3 | 127.7 |
| 4 | 42.9 |
| 5 | 37.4 |
| 6 | 29.5 |
| 7 | 23.7 |
| 8 | 7.5 |

### 5M: Formation of Methylacetate Solvates of Compound (1) (Compound (I).MeOAc)

MeOAc solvates of Compound (1) (Compound (1).MeOAc can be prepared by following the steps described below:

400 mg of Compound (1) was added to 1 mL of MeOAc in a vial. A thick slurry was obtained after vortexing the vial for 1 minute. The resulting mixture was then stirring for 4 hours. The solids from the wet slurry were analyzed by ¹³C SSNMR.

Characteristics of MeOAc solvates of Compound (1): Certain representative peaks of ¹³C SSNMR spectrum of the MeOAc solvates are summarized in Table 33 below.

TABLE 33
Certain representative XRPD of MeOAc Solvate

| ¹³C SSNMR Shifts | ppm (± 0.2) |
|---|---|
| 1 | 170.5 |
| 2 | 137.2 |
| 3 | 106.5 |
| 4 | 54.9 |
| 5 | 51.1 |
| 6 | 21.5 |
| 7 | 21.2 |

### Example 6

### Preparation of Capsules Comprising Polymorphic Form A of Compound (1)

Two different oral dosage formulations of Form A of Compound (1) were prepared as shown in Tables 34a and 34b.

TABLE 34a
200 mg Form A Capsule formulation

| Ingredients | Amounts (mg) | Percent |
|---|---|---|
| Form A of | 200.00 | 52.00 |
| Compound (1) | | |
| Avicel PH 101 | 42.3 | 11.00 |
| Lactose | 53.8 | 14.00 |
| Monohydrate | | |
| Poloxamer 188 | 13.5 | 3.50 |
| Sodium Lauryl | 7.7 | 2.00 |
| Sulfate | | |
| Povidone K29/32 | 19.2 | 5.00 |
| Avicel PH 102 | 11.5 | 3.00 |
| Lactose | 11.5 | 3.00 |
| Monohydrate | | |
| Crosscarmellose | 21.2 | 5.50 |
| Sodium | | |
| Magnesium Stearate | 3.8 | 1.00 |
| Total Formulation | 384.62 | 100.00 |
| Weight (mg) | | |

Final Weight

| | |
|---|---|
| Hard gelatin | 100 |
| Capsule white | |
| opaque, size 0 | |
| Total Weight | 484.62 |

TABLE 3b
50 mg Form A Capsule formulation

| Ingredients | Amounts (mg) | Percent |
|---|---|---|
| Form A of | 50.00 | 11.00 |
| Compound (1) | | |
| Avicel PH 101 | 63.64 | 14.00 |
| Lactose | 172.73 | 38.00 |
| Monohydrate | | |
| Poloxamer 188 | 15.91 | 3.50 |
| Sodium Lauryl | 9.09 | 2.00 |
| Sulfate | | |
| Povidone K29/32 | 22.73 | 5.00 |
| Avicel PH 102 | 36.36 | 8.00 |
| Lactose | 54.55 | 12.00 |
| Monohydrate | | |
| Crosscarmellose | 25.00 | 5.50 |
| Sodium | | |
| Magnesium Stearate | 4.55 | 1.00 |
| Total Formulation | 454.55 | 100.00 |
| Weight (mg) | | |

Final Weight

| | |
|---|---|
| Hard gelatin | 100 |
| Capsule white | |
| opaque, size 0 | |
| Total Weight | 554.55 |

### A. Wet Granulation and Capsule Composition

200 mg Form A capsules were prepared as follows. 50 mg Form A capsules were prepared in a similar manner as described below for 200 mg capsules. The formulation compositions for both the wet granulation and capsules blends of the active capsule are described in Tables 35a and 35b.

TABLE 35a
Polymorphic Form A of Compound (1) (200 mg) Wet
granulation Composition

| | Amount | |
|---|---|---|
| | (mg) | |
| | per | % |
| Component | capsule | W/W |
| Compound (1) crystalline | 200.00 | 59.43 |
| (Form A) | | |
| Avicel PH-101 | 42.31 | 12.57 |
| (microcrystalline | | |
| cellulose), NF, PhEur, JP | | |
| Lactose Monohydrate 80, | 53.85 | 16.00 |
| NF, PhEur, JP | | |
| Poloxamer 188 | 13.46 | 4.00 |
| NF, PhEur, JP | | |
| Sodium Lauryl Sulfate | 7.69 | 2.29 |
| NF, PhEur, JP | | |
| Povidone K29/32 | 19.23 | 5.71 |
| USP | | |
| Total | 336.54 | 100.00 |

TABLE 35b
Polymorphic Form A of Compound (1) (200 mg)
Capsule Composition

| | Amount | |
|---|---|---|
| | (mg) | |
| | per | % |
| Component | capsule | W/W |
| Compound (1) Granulation | 336.54 | 87.50 |
| (Milled) | | |
| Avicel PH-102 | 11.54 | 3.00 |
| (microcrystalline cellulose), | | |
| NF, PhEur, JP | | |
| Lactose Monohydrate 80, | 11.54 | 3.00 |
| NF, PhEur, JP | | |
| Ac-Di-Sol | 21.15 | 5.50 |
| (cross carmellose sodium), | | |
| NF, PhEur, JP | | |
| Magnesium Stearate | 3.85 | 1.00 |
| NF, PhEur, JP | | |
| Total | 384.62 | 100.00 |

The actual weights of each ingredient for the final capsule blend of the 200 mg capsule strength batch can be determined based on the yield calculations of the wet granulation (internal Phase). Sample calculation below:

$$Weight of Excipient = \frac{\begin{matrix}Wet Granulation yield \% \times \\ Theoretical Weight of Excipient ( kg )\end{matrix}}{100}$$

### B. Wet Granulation and Capsule Preparation Overview (200 mg)

### a) High Shear Wet Granulation Process Flow

1. An excess (10%) amount of polymorphic Form A of Compound (1), Avicel PH-101, Lactose Monohydrate, Poloxamer 188, Sodium Lauryl Sulfate, and Povidone K29/32 were weighed.
2. Using the Co-mill equipped with a #20 mesh screen, the excess amount of Compound (I), Avicel PH-101, Lactose Monohydrate, Poloxamer 188, Sodium Lauryl Sulfate, and Povidone K29/32 were screened at 70% speed.
3. The required amount of “sieved” Compound (1), Avicel PH-101, Lactose Monohydrate, Poloxamer 188, Sodium Lauryl Sulfate, and Povidone K29/32 were weighed and transferred to a V-Shell blender (PK 1 cu.ft.).
4. The materials were blended for 5 mins at the set speed (typically 25 RPM).
5. The bulk wet granulation blend was placed in a High shear granulator (Vector GMX.01).
6. The blend was granulated.
7. Once the granulation end point is achieved, the material (Wet granulation blend) was transferred into a suitable container and dried.
8. Using the Co-mill with #20 mesh screen, all the dry granulations was milled.

### b) Capsule Manufacturing Process Flow

9. An excess (10%) amount of Avicel PH-102, Lactose Monohydrate, Crosscarmellose Sodium, and Magnesium Stearate were weighed.
10. Using the Co-mill equipped with a #20 mesh screen, the excess amounts of Avicel PH-102, Lactose Monohydrate, Crosscarmellose Sodium, and Magnesium Stearate were screened at 70% speed.
11. The required amount of “sieved” Avicel PH-102, Lactose Monohydrate, Crosscarmellose Sodium, Magnesium Stearate, and milled granulation were weighed and transferred to a V-Shell blender (PK 1 cu.ft.), except the magnesium stearate.
12. The materials in the V-Shell blender were blended.
13. Magnesium stearate was then added into the V-shell blender, and the mixture was blended.
14. Encapsulate the final blend.

### Example 6

### Preparation of Tablets Comprising Polymorphic Form M of Compound (1)

### a. Tablets A

Wet Granulation and Tablet Composition

The formulation compositions for both the wet granulation and tablet blends of the active tablets are described in Tables 36a and 36b. The overall composition specification of the tablets is described in Table 36c.

TABLE 36a
Form M (250 mg) Wet granulation Composition

| | Amount | |
|---|---|---|
| | (mg) per | % |
| Component | tablet | W/W |
| Compound (1) crystalline | 250.00 | 57.46 |
| (Form M) | | |
| Avicel PH-101 | 52.88 | 12.15 |
| (microcrystalline cellulose), | | |
| NF, PhEur, JP | | |
| Lactose Monohydrate, #316, | 67.31 | 15.47 |
| NF, PhEur, JP | | |
| Poloxamer 188 | 16.83 | 3.87 |
| NF, PhEur, JP | | |
| Sodium Lauryl Sulfate | 9.62 | 2.21 |
| NF, PhEur, JP | | |
| Povidone K12 | 24.04 | 5.53 |
| USP | | |
| Ac-Di-Sol | 14.42 | 3.31 |
| (cross carmellose sodium), | | |
| NF, PhEur, JP | | |
| Total | 435.10 | 100.00 |

TABLE 36b
Form M (250 mg) Tablet Composition

| | Amount | |
|---|---|---|
| | (mg) per | |
| Component | tablet | % W/W |
| Compound (1) Granulation | 435.10 | 78.50 |
| (Milled) | | |
| Avicel PH-102 | 83.14 | 15.00 |
| (microcrystalline cellulose), | | |
| NF, PhEur, JP | | |
| Lactose Monohydrate, #316, | 16.63 | 3.00 |
| NF, PhEur, JP | | |
| Ac-Di-Sol | 13.86 | 2.50 |
| (cross carmellose sodium), | | |
| NF, PhEur, JP | | |
| Magnesium Stearate | 5.54 | 1.00 |
| NF, PhEur, JP | | |
| Total | 554.27 | 100.00 |

TABLE 36c
Form M (250 mg) Tablet Overall Composition

| | | | % in |
|---|---|---|---|
| | | % in dry | core |
| | | granule | tablet |
| intra | Form M of Compound (1) | 57.46 | 45.10 |
| granular | Avicel PH-101, NF, PhEur, JP | 12.15 | 9.54 |
| | Lactose Monohydrate, #316, NF, PhEur, | 15.47 | 12.14 |
| | JP | | |
| | Ac-Di-Sol, NF, PhEur, JP | 3.31 | 2.60 |
| | Sodium Lauryl Sulfate, NF, PhEur, JP | 2.21 | 1.74 |
| | Poloxamer 188, NF, PhEur, JP | 3.87 | 3.04 |
| | Povidone K12, USP | 5.53 | 4.34 |
| | Water, USP | na | na |
| | total granules: | 100.00 | 78.50 |
| extra | Avicel PH-101, NF, PhEur, JP | | 15.00 |
| granular | Lactose Monohydrate, #316, NF, PhEur, | | 3.00 |
| | JP | | |
| | Ac-Di-Sol, NF, PhEur, JP | | 2.50 |
| | Magnesium Stearate, NF, PhEur, JP | | 1.00 |
| | total core tablet: | | 100.00 |

### a) High Shear Wet Granulation Process Flow

1. An excess (10%) amount of Compound (1), Avicel PH-101, Lactose Monohydrate, Poloxamer 188, Sodium Lauryl Sulfate, Povidone K12, and Cross Carmellose Sodium were weighed.
2. Using the Co-mill equipped with an 813 μm mesh screen, the excess amount of Compound (1), Avicel PH-101, Lactose Monohydrate, Poloxamer 188, Sodium Lauryl Sulfate, Povidone K12, and Cross Carmellose Sodium were screened at 30% speed. The sieved materials were placed in individual bags or containers.
3. The required amount of “sieved” Compound (1), Avicel PH-101, Lactose Monohydrate, Poloxamer 188, Sodium Lauryl Sulfate, Povidone K12, and Cross Carmellose Sodium were weighed.
4. A V-Shell blender was set up and the materials from step 3 were transferred into a blender.
5. The materials were blended in the V-Shell blender for 5 mins at the set speed (typically 25 RPM).
6. The contents of the V-Shell blender were emptied into LDPE bags (Bulk Wet Granulation blend).
7. A High shear granulator (Vector GMX.01) with a 1 L granulator bowl was set up.
8. The bulk wet granulation blend was then transferred into the 1 L granulator bowl.
9. The blend was granulated according to the prescribed wet granulation parameters (Table 37)
   Stage 1: 77% of the total amount of water required for the wet granulation was used to granulate the material at the prescribed process parameters. Once the water addition was complete, the granulation was stopped. The walls, impeller, and chopper of the high shear granulator were scraped and the granulation was verified to determine if the visual endpoint was reached. If YES moved on to step 10, if NO proceeded to stage 2
   Stage 2: the remaining 23% of water was added and the material was granulated at the prescribed process parameters. Once the water addition was completed, the granulation was stopped and the walls, impeller, and chopper of the high shear granulator were scraped and the granulation was verified to determine if the visual endpoint was reached. If YES moved on to step 10, if NO continued to granulate at the preceding process parameters with 2 ml portions of water until the end-point was reached.
10. Once the granulation end point was achieved, the material (Wet granulation blend) was screened through a #20 (850 μm) mesh screen and the screened material was transferred into a suitable container.
11. The screened material from step 10 was dried in an oven according to the prescribed drying parameters (overall drying temperature: 30° C.-45° C.).
12. Using the Co-mill with an 813 μm mesh screen, all the dry granulations were milled at 30% speed. (Hand screen any material left over in the Co-mill through a #20 (850 μm) mesh screen, and combine both the milled and screened granulations). The weight of the milled granulation was determined and the material was packaged in bags.

### b) Tablet Manufacturing Process Flow

1. An excess (10%) amount of Avicel PH-102, Lactose Monohydrate, Crosscarmellose Sodium, and Magnesium Stearate were weighed.
2. Using the Co-mill equipped with an 813 μm mesh screen, the excess amounts of Avicel PH-102, Lactose Monohydrate, Crosscarmellose Sodium, and Magnesium Stearate were screened at 30% speed.
3. The required amount of “sieved” Avicel PH-102, Lactose Monohydrate, Crosscarmellose Sodium, Magnesium Stearate, and milled granulation were weighed.
4. The materials were transferred into a V-Shell blender, except the magnesium stearate.
5. The materials in the V-Shell blender were blended for 10 mins at the set speed (typically 25 RPM).
6. The magnesium stearate was then into the V-shell blender.
7. The materials in the V-Shell blender were blended for 1 min at the set speed (typically 25 RPM).
8. The contents of the V-Shell blender were emptied into a bag.
9. A GlobePharma tablet press with the modified caplet tooling (size 0.30″×0.60″) was set up.
10. The final blend was compressed to form tablets.

TABLE 37
Wet granulation process variables, settings and targets

| | | Target Initial |
|---|---|---|
| | Overall | Conditions (or |
| Variable | Setting/Range | Center points) |
| Co-mill speed (% | 10-80% | 30% |
| total speed) | | |
| For 250 mg tablet | 15%-25% of | 18% of |
| Amount of water | granulation | granulation |
| (ml) | blend amount | blend amount |
| Rate of water | N/A | 2.0 ml/min |
| addition (ml/min) | | |

### B. Tablets B

The formulation composition for the pre granulation blend is given in Table 38a. Table 38b gives the composition of the granulation binder solution. The theoretical compression blend composition is given in Table 38c. The composition and approximate batch size of the film coating suspension (including 50% overage for line priming and pump calibration) is given in Table 38d. The overall specification of the tablets B composition is summarized in Table 7e. The target amount of the film coating is 3.0% w/w of the core tablet weight.

TABLE 38a
Pre-granulation composition

| | % |
|---|---|
| Component | W/W |
| Compound (1) crystalline (Form M) | 64.81 |
| Avicel PH-101 (microcrystalline cellulose), NF, PhEur, JP | 13.67 |
| Lactose Monohydrate, #316, NF, PhEur, JP | 17.76 |
| Ac-Di-Sol (cross carmellose sodium), NF, PhEur, JP | 3.76 |
| Total | 100.00 |

TABLE 38b
Binder solution composition

| | % |
|---|---|
| Component | W/W |
| Sodium Lauryl Sulfate, NF, PhEur, JP | 11.75 |
| Poloxamer 188, NF, PhEur, JP | 20.80 |
| Povidone K12, USP | 20.80 |
| Water | 46.65 |
| Total | 100.00 |

TABLE 38c
Compression blend composition

| | % |
|---|---|
| Component | W/W |
| Compound (1) TSWG granulation | 60.20 |
| Avicel PH-101, NF, PhEur, JP | 34.86 |
| Ac-Di-Sol, NF, PhEur, JP | 1.93 |
| Cab-O-Sil MSP, NF, PhEur, JP | 0.60 |
| Sodium Stearyl Fumarate, NF, PhEur, JP | 2.42 |
| Total | 100.00 |

TABLE 38d
Film coat suspension composition

| | % |
|---|---|
| Component | W/W |
| Opadry II White, 85F18378 | 20.00 |
| Water, USP | 80.00 |
| Total | 100.00 |

TABLE 38e
Overall Composition of Tablets B

| | | % in pre- | % in | % in | % in |
|---|---|---|---|---|---|
| | | granulation | dry | core | coated |
| | | blend | granule | tablet | tablet |
| intra | Compound (1) (Form M) | 64.81 | 58.14 | 35.00 | 33.98 |
| granular | Avicel PH-101, NF, PhEur, JP | 13.67 | 12.27 | 7.39 | 7.17 |
| | Lactose Monohydrate, #316, NF, PhEur, | | | | |
| | JP | 17.76 | 15.93 | 9.59 | 9.31 |
| | Ac-Di-Sol, NF, PhEur, JP | 3.76 | 3.37 | 2.03 | 1.97 |
| | total pre-granulation blend: | 100.00 | 89.71 | 54.01 | 52.43 |
| in | Sodium Lauryl Sulfate, NF, PhEur, JP | | 2.27 | 1.37 | 1.33 |
| binder | Poloxamer 188, NF, PhEur, JP | | 4.01 | 2.42 | 2.34 |
| solution | Povidone K12, USP | | 4.01 | 2.42 | 2.34 |
| | Water, USP | | na | na | na |
| | total granules: | | 100.00 | 60.20 | 58.45 |
| extra | Avicel PH-101, NF, PhEur, JP | | | 34.86 | 33.85 |
| granular | Ac-Di-Sol, NF, PhEur, JP | | | 1.93 | 1.87 |
| | Cab-O-Sil MSP, NF, PhEur, JP | | | 0.60 | 0.58 |
| | Sodium Stearyl Fumarate, NF, PhEur, JP | | | 2.42 | 2.34 |
| | total core tablet: | | | 100.00 | 97.09 |
| coating | Opadry II White, 85F18378 | | | | 2.91 |
| | Water, USP | | | | na |
| | total final tablet: | | | | 100.00 |

### A. Wet Granulation

a) Binder Solution Preparation
The binder solution included the Povidone, SLS, and Poloxamer. The solution was prepared based on 9% w/w water content of the final dry granulation. An excess amount of 100% was prepared for pump calibration, priming lines, etc.
1. The required amount of Poloxamer 188, Sodium Lauryl Sulfate, Povidone K12, and purified (DI) water were weighed.
2. Under constant stirring, was add the Povidone K12 to the DI water, and the resulting mixture was stirred. Poloxamer 188 and Sodium Lauryl Sulfate were added into the tank containing the DI water and dissolved Povidone K12. The stir rate was then turned down after the surfactant addition such that only a partial vortex formed.
3. The solution was stirred until all the solids present were visually fully dissolved.
4. The solution was then sat at least 2 hours until air bubbles in solution disappeared. Alternatively, a partial vacuum could be pulled on the solution tank for up to an hour to degas the solution.

### b) Wet Granulation Process

1. Compound (1), Croscarmellose Sodium, Avicel PH-101, and Lactose Monohydrate were weighed.
2. Using a U5 or U10 Comill equipped with a 32R screen and round impeller, the weighed out Compound (1), lactose, and avicel were delumped respectively at 4000 rpm in the U5, or 2800 rpm in the U10 into bags or directly into the Meto 200 L blender.
3. The materials were transferred from step 2 into a Meto 200 L bin blender.
4. The materials were blended for 25 minutes at 10 RPM.
5. The materials were charged into a loss in weight powder feeder directly from the blend shell, or into a LDPE bag.
6. A Leistritz 27 mm twin screw extruder with the required barrel and screw configuration specified in Tables 39a and 39b were set up.
7. The dry blend was fed into the extruder using a K-Tron loss in weight feeder.
8. The binder fluid was injected into the extruder using a calibrated K-Tron liquid pump. The pump was calibrated using the actual fluid prior to operation.
9. The blend was then granulated.
10. The weight ratio of solution feed rate over powder feed rate was 0.215 to have the proper final composition. For the intended powder feed of 167.00 g min⁻¹, the solution feed rate was 35.91 g min⁻¹.
11. The wet granules coming out of the twin screw was milled using an inline U5 Comil at 1000 rpm with square 4 mm screen and round bar impeller.
12. The wet milled granules were collected and dried. The water content was NMT 3.0%.

TABLE 39a
27-mm Leistritz Twin Screw Extruder barrel configuration

| Barrel Number | Barrel Configuration |
|---|---|
| 1 | Blank, or Plugged Vent |
| 2 | Blank, or Plugged Vent |
| 3 | Blank, or Plugged Vent |
| 4 | Blank, or Plugged Vent |
| 5 | Blank, or Plugged Vent |
| 6 | Feed |
| 7 | Liquid injection (nozzle orifice is 0.7 mm) |
| 8 | Blank, or Plugged Vent |
| die config | no die |

TABLE 39b
27-mm Leistritz Twin Screw Extruder screw configuration
Screw configuration (tail to tip)

Spacers for rest of screw shaft
GFA-2-30-90
GFA-2-30-90
GFA-2-30-30
GFA-2-20-90
2-row, 5-tooth per row combing element
GFA-2-30-60
Tip

### B. Extra-Granular Blending and Compression Process

1. The quantity of the extra-granular excipients based on the compression blend composition was weighed.
2. The granules and Cab-O—Sil was added directly to the 200 L Meto bin blender and blended for 8 minutes at 15 RPM.
3. The blend was then passed through a U10 Comil with a 40G screen and round bar impeller at 600 rpm directly into the 600 L Meto bin blender or into double LDPE bags.
4. Approximate amounts of Avicel PH-101 and Ac-Di-Sol were screened using a U10 Comil with a 32R screen and round bar impeller at 600 rpm directly into the 600 L Meto bin blender or into double LDPE bags.
5. Sodium stearyl (SSF) was hand screened through a #50 mesh screen into an appropriate container. A portion of the extra granular blend equal to roughly 10 times by mass the amount of SSF calculated in step one was placed in the container with the SSF and blended for 30 seconds before the mixture was added to the bin blender.
6. The mixture was blended for 10 minutes at 15 rpm.
7. The final blend was compressed.
8. During the compression process, the individual and average tablet weights, hardness, and thickness were measured.

### C. Film Coating Process

A film coating was applied to the core tablets in a Vector VPC 1355 pan coater as a 20 wt % Opadry II white #85F18378 aqueous suspension. The target coating was 3.0% w/w of the core tablet weight, with an acceptable range of 2.5% to 3.5%. To accomplish this, an amount of coating suspension equivalent to a 3.2% weight gain was sprayed, which would give a 3.0% coating assuming a coating efficiency of 95%. The film coating process was performed as follows:
1. Calculate the pan load by dividing the tablet yield by 3 (or 2 if there are less than 75 kg of core tablets) and calculate the required amount of coating suspension (based on 3.2% coating), including 50% overage for line priming, pump rate testing, and coating pan walls.
2. Prepare the coating suspension by slowly adding the Opadry II #85F18378 powder to the appropriate amount of DI water while continuously stirring the fluid with an overhead stirrer, ensuring sufficient wetting of the powder. Once all Opadry is added to the water, continue stirring at a low rpm for 60 minutes. The maximum hold time for the spray suspension is 24 hours.
3. Pre-coat the pan with Opadry by spraying the coating suspension for 5 to 10 minutes. After spraying dry the pan for 1 to 2 minutes.
4. Load the calculated amount of tablets in the coating pan.
5. Pre-heat the pan to the required bed temperature while jogging the pan. Calculate the tablet weight gain and confirm that the coating amount is between 2.5% and 3.5%. Stop spraying once that amount is sprayed. When coating amount is sufficient, dry the tablets for an additional 5 minutes. Turn the heating off and allow the tablets to cool while jogging the pan. When the bed temperature reaches 35° C. (±1° C.), the process is stopped. The coating pan door was remained closed during the cool down period.

### Example 7

### Tablets of Tromethamine Salt of Compound (1)

The formulation compositions for both the wet granulation and tablet blends of the active tablets are described in Tables 40a and 40b. The overall specification of the tromethamine salt tablets is described in Table 40c.

TABLE 40a
Tromethamine Salt of Compound (1) (250 mg)
Wet granulation Composition

| | Amount | |
|---|---|---|
| | (mg) | |
| | per | % |
| Component | tablet | W/W |
| Compound (1) | 319.57 | 45.00 |
| (Tromethamine Salt) | | |
| Avicel PH-101 | 221.50 | 31.19 |
| (microcrystalline cellulose), | | |
| NF, PhEur, JP | | |
| Lactose Monohydrate, #316, | 86.07 | 12.12 |
| NF, PhEur, JP | | |
| Poloxamer 188 | 21.52 | 3.03 |
| NF, PhEur, JP | | |
| Sodium Lauryl Sulfate | 12.29 | 1.73 |
| NF, PhEur, JP | | |
| Povidone K12 | 30.75 | 4.33 |
| USP | | |
| Ac-Di-Sol | 18.46 | 2.60 |
| (cross carmellose sodium), | | |
| NF, PhEur, JP | | |
| Total | 710.16 | 100.00 |

TABLE 40b
Tromethamine Salt of Compound (1) (250 mg) Tablet Composition

| | Amount | |
|---|---|---|
| | (mg) | |
| | per | % |
| Component | tablet | W/W |
| Compound (1) Granulation | 710.16 | 78.49 |
| (Milled) | | |
| Avicel PH-102 (microcrystalline | 135.7 | 15.00 |
| cellulose), NF, PhEur, JP | | |
| Lactose Monohydrate, #316, | 27.14 | 3.00 |
| NF, PhEur, JP | | |
| Ac-Di-Sol | 22.62 | 2.50 |
| (cross carmellose sodium), NF, | | |
| PhEur, JP | | |
| Magnesium Stearate | 9.05 | 1.00 |
| NF, PhEur, JP | | |
| Total | 904.67 | 99.99 |

TABLE 40c
Overall Composition of Tromethamine Salt of
Compound (1) (250 mg) Tablet

| | | | % in |
|---|---|---|---|
| | | % in dry | core |
| | | granule | tablet |
| intra | Compound (1) (Tromethamine salt) | 45.00 | 35.32 |
| granular | Avicel PH-101, NF, PhEur, JP | 31.19 | 24.48 |
| | Lactose Monohydrate, #316, NF, PhEur, | 12.12 | 9.51 |
| | JP | | |
| | Ac-Di-Sol, NF, PhEur, JP | 2.60 | 2.04 |
| | Sodium Lauryl Sulfate, NF, PhEur, JP | 1.73 | 1.36 |
| | Poloxamer 188, NF, PhEur, JP | 3.03 | 2.38 |
| | Povidone K12, USP | 4.33 | 3.40 |
| | Water, USP | na | na |
| | total granules: | 100.00 | 78.50 |
| extra | Avicel PH-101, NF, PhEur, JP | | 15.00 |
| granular | Lactose Monohydrate, #316, NF, PhEur, | | 3.00 |
| | JP | | |
| | Ac-Di-Sol, NF, PhEur, JP | | 2.50 |
| | Magnesium Stearate, NF, PhEur, JP | | 1.00 |
| | total core tablet: | | 100.00 |

The actual weights of each ingredient for the final tablet blend of the 250 mg tablet strength batch can be determined based on the yield calculations of the wet granulation (internal Phase). Sample calculation below

$$Weight of Excipient = \frac{\begin{matrix}Wet Granulation yield \% \times \\ Theoretical Weight of Excipient ( kg )\end{matrix}}{100}$$

### a) High Shear Wet Granulation Process Flow

1. An excess (10%) amount of Tromethamine salt of Compound (1), Avicel PH-101, Lactose Monohydrate, Poloxamer 188, Sodium Lauryl Sulfate, Povidone K12, and Cross Carmellose Sodium were weighed.
2. Using the Co-mill equipped with a #20 mesh screen (or hand screen), the excess amount of Compound (1), Avicel PH-101, Lactose Monohydrate, Poloxamer 188, Sodium Lauryl Sulfate, Povidone K12, and Cross Carmellose Sodium were screened at 70% speed.
3. The required amount of the sieved Compound (1), Avicel PH-101, Lactose Monohydrate, Poloxamer 188, Sodium Lauryl Sulfate, Povidone K12, and Cross Carmellose Sodium were weighed.
4. The materials from step 3 were transferred into a V-Shell blender.
5. The materials in the V-Shell blender were blended for 5 mins at the set speed (typically 25 RPM).
6. The contents of the V-Shell blender were emptied into LDPE bags (Bulk Wet Granulation blend).
7. The bulk wet granulation blend from step 6 was placed into a high shear granulator (Vector GMX.01) with a 1 L granulator bowl.
8. The blend was granulated. The wet granulation process was performed in two stages:
   Stage 1: 77% of the total amount of water required for the wet granulation was used to granulate the material at the prescribed process parameters. Once the water addition was complete, the granulation was stopped, and the walls, impeller, and chopper of the high shear granulator were scraped and the granulation was verified to determine if the visual endpoint was reached. If YES moved on to step 10, if NO proceeded to stage 2
   Stage 2: The remaining 23% of water was added and the material was granulated. Once the water addition was complete, the granulation was stopped, and the walls, impeller, and chopper of the high shear granulator were scraped and the granulation was verified to determine if the visual endpoint was reached.
   Stage 3: The material was granulated at the prescribed process parameters using just the impellor and chopper for ˜30 seconds. The granulation was stopped, and the walls, impeller, and chopper of the high shear granulator were scraped and the granulation was verified to determine if the visual endpoint was reached. If YES moved on to next step, if NO continued to granulate at the preceding process parameters (Stage 2) with 2 ml portions of water until the end-point was reached. Once the granulation end point was achieved, the material (Wet granulation blend) was screened through a #10 mesh screen and the screened material was transferred into a suitable container.
9. The material was then dried.
10. Once the material was confirmed dried, using a #20 (850 μm) mesh screen, hand all the dry granulations were careened.

### b) Tablet Manufacturing Process Flow

11. An excess (10%) amount of Avicel PH-102, Crosscarmellose Sodium, and Magnesium Stearate were weighed.
12. Using the Co-mill equipped with an 813 μm mesh screen (or hand screening), the excess amounts of Avicel PH-102, Crosscarmellose Sodium, and Magnesium Stearate were screened at 30% speed, and the sieved materials were placed in individual bags or containers.
13. The required amount of “sieved” Avicel PH-102, Crosscarmellose Sodium, Magnesium Stearate, and milled granulation were weighed.
14. The materials from previous step, except the magnesium stearate, were transferred into a V-Shell blender.
15. The materials in the V-Shell blender were blended for 5 mins at the set speed (typically 25 RPM).
16. Magnesium stearate was then added into the V-shell blender.
17. The resulting materials in the V-Shell blender were blended for 1 min at the set speed (typically 25 RPM).
18. The final blend was compressed using a GlobePharma tablet press according to the prescribed tablet compression process parameters. During the compression process, the individual and average tablet weights, hardness, thickness, and friability were monitored.
19. At the end of the run, all the tablets were dedusted and placed into bottles.

### Example 8

### IV Formulation of Compound (1)

A description of the manufacturing process is provided below.

TABLE 41
Quantitative Batch Formula for Form M IV Solution

| Ingredient | mg/mL |
|---|---|
| Form M of Compound (1) | 5.00 |
| Hydroxypropyl-β-cyclodextrin, | 25.0 |
| HPβCD | |

70 mM Phosphate Buffer, pH 7.4, 12 L

| | |
|---|---|
| Sodium Phosphate, monobasic, | 2.182 |
| monohydrate | |
| Sodium Phosphate, dibasic, | 14.523 |
| heptahydrate | |
| Dextrose, Anhydrous | 25.0 |
| WFI | qs |

E. Sterilization of all the equipment and components to be used in the process was performed.
F. 10% Phosphoric Acid and 1M Sodium Hydroxide solution was prepared for pH adjustment
   a. 10% Phosphoric Acid (received as 86%):
      Approximately 250 mL of Water for Injection (WFI) was added to a 500 mL volumetric flask. Then 59 mL of phosphoric acid was slowly added to the flask. The mixture was then mixed.
   b. 1 M Sodium Hydroxide:
      Approximately 250 mL of WFI was added to a 500 mL volumetric flask. Then 20 g of Sodium Hydroxide was slowly added to the flask. The mixture was then mixed.
G. 70 mM phosphate buffer with dextrose was prepared—12 L
   a. The required quantities of dextrose, mono and dibasic sodium phosphate were weighed.
   b. Approximately 10 L of cool WFI (15-30° C.) was added to the compounding vessel.
   c. The mixture was then mixed.
   d. The weighed quantities of dextrose, mono and dibasic sodium phosphate, were added into the vessel. The mixture was then mixed until solution is clear.
   e. A 10 mL sample was taken for checking pH. If necessary, the pH was adjusted to have pH 7.4 (range: 7.2 to 7.6) with 10% Phosphoric Acid or 1 M Sodium Hydroxide Solution.
   f. QS to 12 L (12.2 kg, given the density of 1.013 g/mL) with WFI (15-30° C.). Mix for NLT 5 minutes.
H. Prepare Compound (1)/HPIβCD solution
   a. The required quantities of HPIβCD and Form M of Compound (1) were weighed.
   b. Approximately 9 kg of phosphate/dextrose buffer (15-30° C.) was added to compounding vessel with stir bar.
   c. The weighed HPIβCD was added to the buffer solution and the mixture was stirred for NLT 5 minutes until the solution became clear.
   d. Compound (1) was then added into the compounding vessel. The vessel walls above the fluid were rinsed with 50-100 mL of buffer solution to wash down any residual drug that might be on the sides. The resulting mixture was then mixed for NLT 2 hours until the solution became clear.
   e. A 10 mL sample was taken and checked for pH. If necessary, the pH was adjusted to have pH 7.0 (range: 7.0 to 7.4) with 10% Phosphoric Acid or 1 M Sodium Hydroxide Solution.
   f. QS to 10 L (10.2 kg, given the density of 1.0218 g/mL) with phosphate/dextrose buffer (15-30° C.). Mix for NLT 5 minutes.
I. The bulk solution was filtered through 2, Millipak 200, 0.22 micron filters in series, into a sterile 20 L Flexboy bag using a peristaltic pump.
J. Using the Flexicon peristaltic filler, the solution was placed into vials. The filled vials were stored at 15-30° C.

### Example 9

### Preparation of Additional Tablets Comprising Polymorphic Form M of Compound (1)

### a. Tablets C

Roller Compaction and Tablet Composition

The overall composition specification of the tablets is described in Table 42. The tablet formulation was prepared in a similar manner as described above in Example 8 but using roller compaction instead of twin screw wet granulation process. In short, the manufacturing process includes:

Compound (1) (Form M), Microcrystalline cellulose, and croscarmellose sodium were individually screened, added to the blender and blended. Magnesium stearate was individually screened, added to the above blend and further blended. The blend was then dry granulated using a roller compactor and milled into granules. The granules were then further blended with individually screened Microcrystalline cellulose, croscarmellose sodium and sodium stearyl stearate. The final blend was then compressed into tablets. The final tablet contained 400 mg of Compound (1). Following the compression, SDD tablets were tested for release and packaged.

TABLE 42
Form M Tablet C Overall Composition
TSWG Granulation

| | Amount |
|---|---|
| | per |
| | Tablet |
| | (mg) |
| Form M of Compound (1) | 400 |
| Avicel PH-102 | 42.2 |
| Lactose Monohydrate | 0.0 |
| Ac-Di-Sol | 23.2 |
| Magnesium Stearate | 5.0 |
| total granules: | 470.4 |
| Avicel PH-101 | 192.1 |
| Ac-Di-Sol | 0.9 |
| Magnesium Stearate | 3.0 |
| Total | 666.4 |

### B. Tablets D

Wet Granulation and Tablet Composition

The tablet formulation was prepared in a similar manner, using Consigma 1 twin screw granulator with Fluid bed dryer, as described above in Example 8 for Tablet B. The overall Compound (1) granule composition tablet for HPC 2.25% is given in Table 43a and 43b.

The tablet formulation was prepared in a similar manner, using Consigma 1 twin screw granulator with Fluid bed dryer, as described above in Example 6 for Tablet B. The overall Compound (1) granule composition tablet for HPC 2.25% is given in Table 19a and 19b.

TABLE 43a
Form M Tablet D Overall Composition
TSWG Granulation

| | Amount | | |
|---|---|---|---|
| | per | | |
| | Tablet | Amount in | Target |
| | (mg) | granulation | (g) |
| Form M of Compound (1) | 400 | 88.26 | 88.26 |
| Avicel PH-101 | 8.6 | 1.90 | 1.90 |
| Lactose Monohydrate | 11.2 | 2.47 | 2.47 |
| HPC-SL | 10.2 | 2.25 | 2.25 |
| Crosscarmellose Sodium | 23.2 | 5.12 | 5.12 |
| total granules: | 453.2 | 100.0 | 100.0 |

Tablet Blend

| | Amount | | | |
|---|---|---|---|---|
| | per | | | |
| | Tablet | Granulation | Target | Target |
| | (mg) | (%) | (%) | (g) |
| Form M of Compound (1) Granulation | 453.2 | 100.0 | 62.32 | 21.81 |
| (Milled) | | | | |
| Avicel PH-101 | 237.8 | 52.8 | 32.69 | 11.44 |
| Sodium Stearyl Fumarate | 21.8 | 4.45 | 3.00 | 1.05 |
| Crosscarmellose Sodium | 14.5 | 3.2 | 1.99 | 0.70 |
| total granules: | 727.3 | 100.0 | 100.00 | 35.00 |

TABLE 43b
Other Overall Compositions Form M Tablet D

| | | | | | | wt % in |
|---|---|---|---|---|---|---|
| | | | | wt % in | | coated |
| | wt % in pre- | wt % in dry | wt % in | coated | Amount in | tablet in |
| | granulation | granulation | core tablet | tablet | tablet (mg) | ranges |
| Compound (1) | 90.29 | 88.04 | 55.00 | 53.40 | 400 | 50-60 |
| (Form M) | | | | | | |
| Avicel 101 | 1.94 | 1.89 | 1.18 | 1.15 | 8.6 | 1-2 |
| Lactose | 2.53 | 2.47 | 1.54 | 1.50 | 11.2 | 1-2 |
| Monohydrate | | | | | | |
| Crosscarmellose | 5.24 | 5.11 | 3.19 | 3.10 | 23.2 | 2-4 |
| Sodium | | | | | | |
| | 100 | 97.50 | 60.91 | 59.14 | 443 | |
| HPC-SL | | 2.50 | 1.56 | 1.52 | 11.36 | 1-3 |
| Water | | 0 | 0 | 0 | 0 | |
| | 100 | 100 | 62.47 | 60.65 | 454.36 | |
| Avicel 101 | | | 32.53 | 31.58 | 236.56 | 25-35 |
| Crosscarmellose | | | 2.00 | 1.94 | 14.54 | 1-3 |
| Sodium | | | | | | |
| Sodium Stearyl | | | 3.00 | 2.91 | 21.82 | 2-4 |
| Fumarate | | | | | | |
| | | | 100 | 97.09 | 727.27 | |
| Opadry II | | | | 2.91 | 21.82 | |
| Water | | | | 0 | 0 | |
| | | | | 100 | 749.09 | 100 |

The formulation composition and batch size for the pre granulation blend was given in Table 44a. Tables 44b, c, d, e, f and g gave the composition and batch size of the granulation binder solutions. The batch size of the binder solutions included a 100% overage for pump calibration and priming of solution lines.

TABLE 44a
Pre granulation composition and batch size

| | | Quantity |
|---|---|---|
| | % | per batch |
| Component | W/W | (kg) |
| Compound (1) crystalline (Form M) | 90.29 | 8.80 |
| Avicel PH-101 (microcrystalline cellulose), NF, | 1.94 | 0.19 |
| PhEur, JP | | |
| Lactose Monohydrate, #316, NF, PhEur, JP | 2.53 | 0.25 |
| Ac-Di-Sol (cross carmellose sodium), NF, PhEur, JP | 5.24 | 0.51 |
| Total | 100.00 | 9.75 |

TABLE 44b
HPC (1.5%) Binder solution composition and batch size (48% water)

| | % | Batch size |
|---|---|---|
| Component | W/W | (g) |
| HPC-SL, USP | 3.03 | 30.3 |
| Water | 96.97 | 969.7 |
| Total | 100 | 1000 |

TABLE 44c
HPC (2.5%) Binder solution composition and batch size (48% water)

| | % | Batch size |
|---|---|---|
| Component | W/W | (g) |
| HPC-SL, USP | 4.95 | 49.5 |
| Water | 95.05 | 950.5 |
| Total | 100 | 1000 |

TABLE 44d
HPC (1.5%) Binder solution composition and batch size (58% water)

| | % | Batch size |
|---|---|---|
| Component | W/W | (g) |
| HPC-SL, USP | 2.52 | 25.2 |
| Water | 97.48 | 974.8 |
| Total | 100 | 1000 |

TABLE 44e
HPC (2.5%) Binder solution composition and batch size (58% water)

| | % | Batch size |
|---|---|---|
| Component | W/W | (g) |
| HPC-SL, USP | 4.13 | 41.3 |
| Water | 95.87 | 958.7 |
| Total | 100 | 1000 |

TABLE 44f
HPC (2.0%) Binder solution composition and batch size (53% water)

| | % | Batch size |
|---|---|---|
| Component | W/W | (kg) |
| HPC-SL, USP | 3.63 | 145.2 |
| Water | 96.37 | 3854.8 |
| Total | 100 | 4000 |

TABLE 44g
HPC (2.25%) Binder solution composition and batch size (53% water)

| | % | Batch size |
|---|---|---|
| Component | W/W | (kg) |
| HPC-SL, USP | 4.07 | 162.8 |
| Water | 95.93 | 3837.2 |
| Total | 100 | 4000 |

a) Binder Solution Preparation (HPC 1.5%-2.5%)
The binder solution included the HPC binder. The solution was prepared based on 48, 53, and 58% w/w water content of the final dry granulation. An excess amount of 100% was prepared for pump calibration, priming lines, etc.
1. Weigh out the required amounts (Table 44b, c, d, e, f, and g) of HPC, and purified (DI) water.
2. Under constant stirring add the HPC-SL to the DI water and stir until fully dissolved. Turn down the stir rate such that only a partial vortex forms.
3. Stir the solution until all the solids present are visually fully dissolved.
4. Cover and let the solution sit for 2-4 hours until air bubbles in solution have disappeared. Alternatively, a partial vacuum can be pulled on the solution tank for up to an hour to degas the solution.

b) Wet Granulation Process
1. Weigh the correct amounts of Compound (1), Croscarmellose Sodium, Avicel PH-101, and Lactose Monohydrate per Table 44a.
2. Using a U5 or U10 Comill equipped with a 32R screen and round impeller, delump the weighed out Compound (1), Lactose, and Avicel respectively at 4000 rpm in the U5, or 2800 rpm in the U10 into a bag or directly into the Bin blender.
3. Set up the blender and transfer the materials from step 2 into the blender if the material was delumped into a bag.
4. Blend the materials for 5 minutes at 23 RPM. Based on a bulk density of 0.4-0.5 g cc⁻¹, the blender should be 59%-74% full.
5. Take two ×1.0 g samples, one for Karl Fischer (KF) and the other for LOD testing. These samples do not have to be taken with the sample thief
6. Charge 5 kg of the pre granulation blend into the loss in weight powder feeder directly from the blend shell. Empty the remainder of the blender contents into labeled LDPE bags or charge directly from the blend shell into the Loss in Weight feeder.
7. Set up the Consigma 1 twin screw granulator with the standard screw configuration as specified in Table 45.
8. Feed the dry blend into the extruder using the Barbender loss in weight feeder.
9. Inject the binder fluid into the granulator using the calibrated liquid pump.
10. Granulate the blend according to the prescribed experimental design shown in Table 46
11. Granulate approximately 4 kg of material for experiments 1-4 (1 kg per experiment), and approximately 6 kg of material for experiments 5 and 6 (3 kg per experiment)
12. The weight ratio of solution feed rate over powder feed rate varies from one experiment to the other (see the solution federates in Table 45 for all the experiments when the powder federates are kept constant at 167 g/min).
13. Collect the granules from each experiment into separate LDPE bags

c) Fluid Bed Drying process
14. Charge approximately 1 kg of granules into the fluid bed dryer and dry according to the parameters shown in Table 46.
15. Collect the dried granules into separate LDPE bags.

TABLE 45
Granulation Experiment design

| | | | | Granule | Solution |
|---|---|---|---|---|---|
| | Water | HPC | | DL | Feed Rate |
| Experiment | (%) | (%) | DOE | (%) | (g/min) |
| 1 | 48 | 1.50 | −− | 88.94 | 83.77 |
| 2 | 48 | 2.50 | −+ | 88.04 | 86.34 |
| 3 | 58 | 1.50 | +− | 88.94 | 100.76 |
| 4 | 58 | 2.50 | ++ | 88.04 | 103.44 |
| 5 | 53 | 2.00 | 00 | 88.49 | 93.56 |
| 6 | 53 | 2.25 | N/A | 88.26 | 94.22 |

TABLE 46
Process Control Parameters

| | |
|---|---|
| Parameter | Target Value and range |

Blending Operations

| | |
|---|---|
| Pre granulation blending | 252 (+−10 revolutions) |

Twin Screw Wet Granulation

| | |
|---|---|
| Screw configuration | K/6, 60\|XT\|6K/4, 60\|1.5T\| |
| | 6K/4, 60\|1.5T\|2K/6, 60 |
| Powder Feed Rate | 167.0 g/min (+/−0.5%) |
| Liquid Feed Rate (0.8 mm nozzle) | 83-103 g/min (+/−0.5%) |
| Screw Speed | 400 RPM (+/−10 RPM) |
| Barrel Temperature | 25° C. (range 20-30° C.) |

Granule Drying

| | |
|---|---|
| Inlet Air Temperature | 50° C. (range 45-55° C.) |
| Inlet Air flow | 30-100 m³/hr |
| Drying Time | 10-20 mins |

All references provided herein are incorporated herein in its entirety by reference. As used herein, all abbreviations, symbols and conventions are consistent with those used in the contemporary scientific literature. See, e.g., Janet S. Dodd, ed., The ACS Style Guide: A Manual for Authors and Editors, 2nd Ed., Washington, D.C.: American Chemical Society, 1997.

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of preparing Compound (1) represented by the following structural formula: 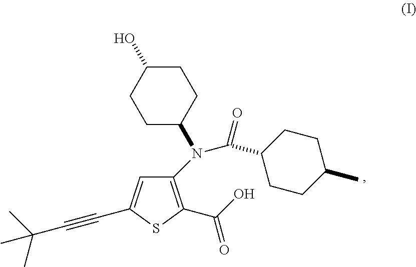
or a pharmaceutically acceptable salt thereof, comprising:
a) reacting Compound (A) with 3,3-dimethylbut-1-yne in the presence of one or more palladium catalysts selected from the group consisting of Pd(PPh₃)₄ and Pd(PPh₃)₂Cl₂, and one or more copper catalysts selected from the group consisting of Cut, CuBr, and CuCl to generate Compound (B); 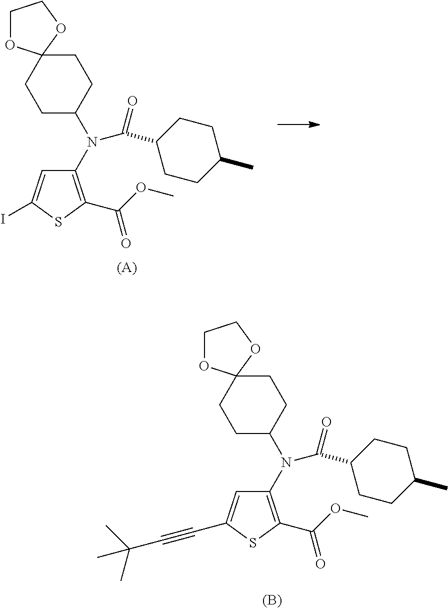
b) treating Compound (B) with an acid to generate Compound (C): 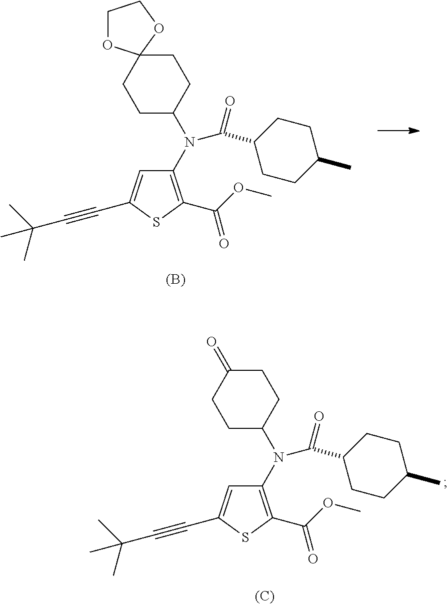
c) reducing the cyclohexanone of Compound (C) to cyclohexanol to generate Compound (D): 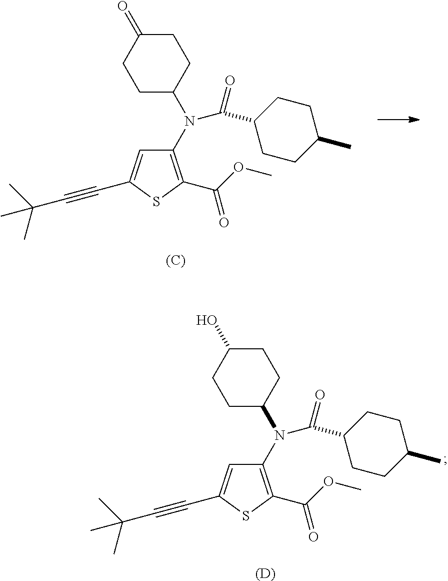
and
d) reacting Compound (D) with a base to generate Compound (1).

2. The method of claim 1 wherein the reaction of Compound (A) and 3,3-dimethylbut-1-yne in the presence of said one or more palladium and copper catalysts in step a) is performed in the presence of Et₃N or ⁱPr₂NH, wherein Et is ethyl and ⁱPr is propyl.

3-7. (canceled)

8. The method of claim 1 wherein the reaction of Compound (A) and 3,3-dimethylbut-1-yne in the presence of said one or more palladium and copper catalysts in step a) is performed at a temperature in a range of 18° C.-30° C.

9. (canceled)

10. The method of claim 1, wherein the reaction of Compound (A) and 3,3-dimethylbut-1-yne in the presence of said one or more palladium and copper catalysts in step a) is performed in a solvent system that includes 2-methyl tetrahydrofuran, 2-butanone, or methyl t-butyl ether.

11. (canceled)

12. The method of claim 1, wherein the acid for step b) is HCl.

13-15. (canceled)

16. The method of claim 1 wherein the reduction of the cyclohexanone of Compound (C) to cyclohexanol is carried out by the use of LiAlH(O^{t}Bu)₃, wherein ^{t}Bu is tert-butyl.

17. The method of claim 1 wherein the base of step d) is selected from NaOH, LiOH, Bu₄NOH, or NaOMe, or a combination thereof, wherein Bu is n-butyl and Me is methyl.

18. (canceled)

19. The method of claim 1 further comprising crystallization of Compound (C) from a mixture of acetone, 2-butanone, and water prior to step c).

20-22. (canceled)

23. The method of claim 19, wherein the crystallization of Compound (1) is performed: in ethylacetate at a temperature in a range of 45° C. to 47° C.; in n-butylacetate at a temperature in a range of 35° C. to 47° C.; or in a mixture of n-butyl acetate and acetone at a temperature in a range of 30° C. to 47° C.

24. The method of claim 1 further comprising preparing Compound (A) by reacting Compound (E) with I₂:

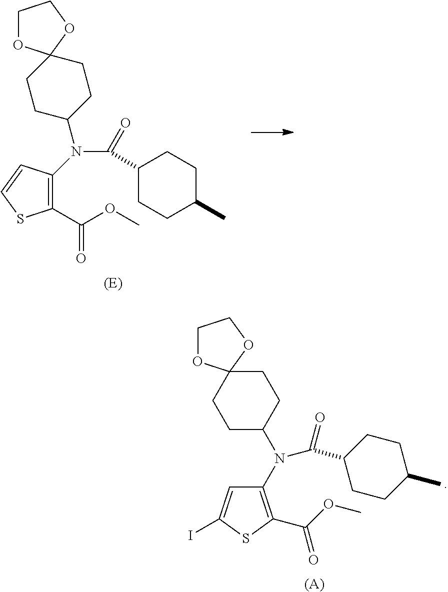

25. (canceled)

26. The method of claim 24 further comprising the step of preparing Compound (E) by amidating Compound (G) with Compound (F):

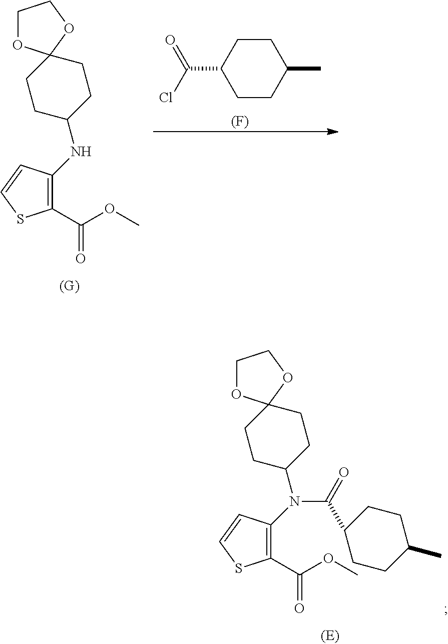

27. The method of claim 26 wherein Compound (F) is provided in situ by reacting Compound (H): 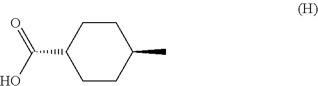
with SOCl₂.

28. (canceled)

29. (canceled)

30. The method of claim 26, further comprising the step of preparing Compound (G) by reacting Compound (J) with Compound (K):

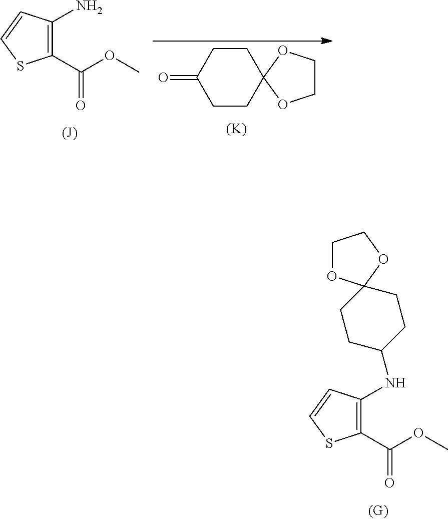

31-36. (canceled)

37. A method of preparing Compound (1) represented by the following structural formula: 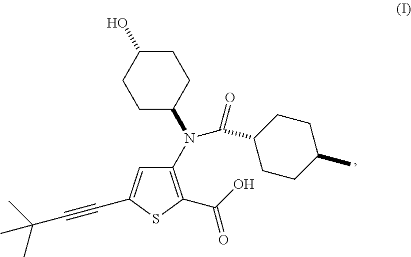
or a pharmaceutically acceptable salt thereof, comprising:
a) reducing the cyclohexanone of Compound (C) to cyclohexanol in the presence of LiAlH(O^{t}Bu)₃ in an amount of 1.0 to 1.5 equivalents based on molar amount of Compound (C) at a temperature in a range of −70° C. to −35° C. to generate Compound (D): 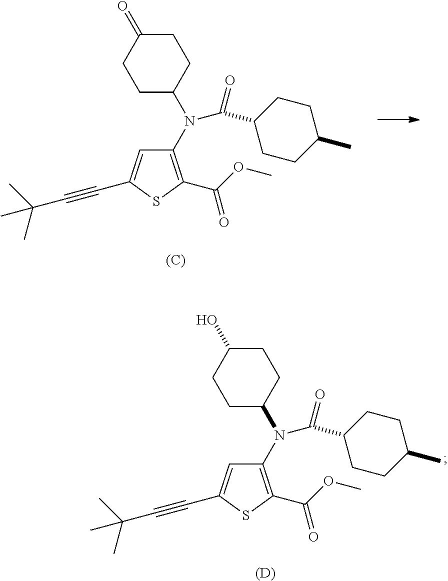
and
iii) reacting Compound (D) with a base to generate Compound (1), wherein ^{t}Bu is t-butyl.

38. (canceled)

39. The method of claim 37 wherein the step a) is performed in a solvent system that includes THF and/or 2-MeTHF.

40. The method of claim 37, further comprising the step of preparing Compound (C) by treating Compound (B) with an acid to generate Compound (C):

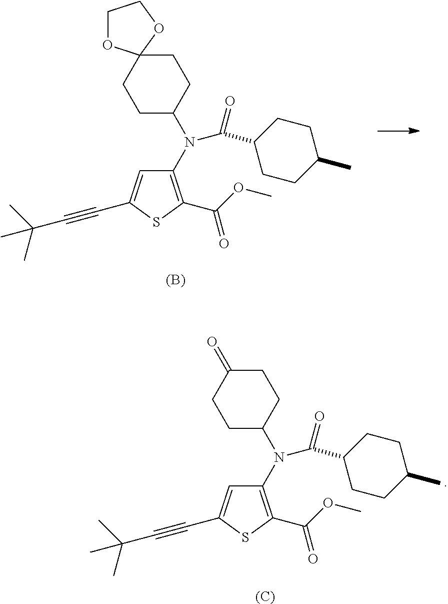

41. The method of claim 40 wherein the acid is HCl.

42. (canceled)

43. A method of preparing Compound (1) represented by the following structural formula: 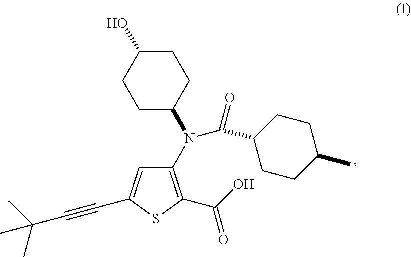
or a pharmaceutically acceptable salt thereof, comprising:
a) reacting Compound (J) with Compound (K) to produce Compound (G) by combining them with NaBH(OAc)₃ and trichloroacetic acid, wherein Ac is acetyl: 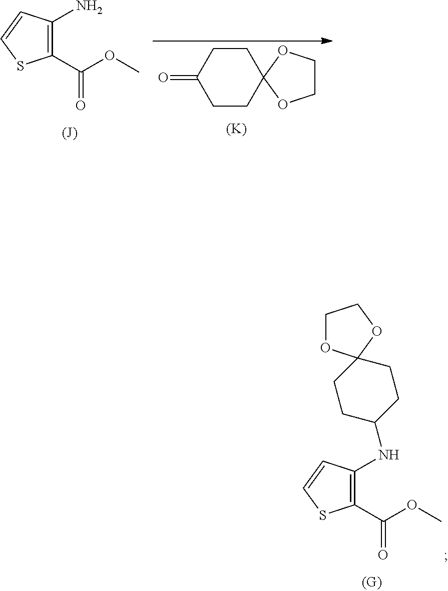
b) amidating Compound (G) with Compound (F) to produce Compound (E): 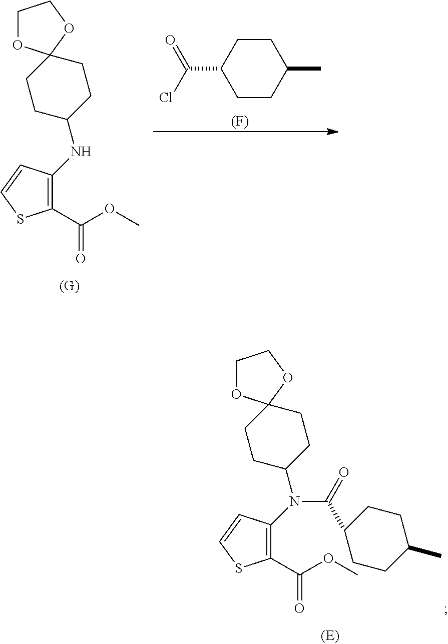
c) reacting Compound (E) with I₂ to produce Compound (A): 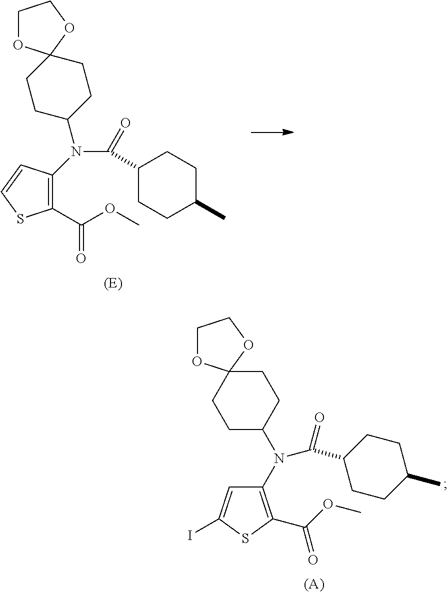
d) reacting Compound (A) with 3,3-dimethylbut-1-yne in the presence of one or more palladium catalysts selected from the group consisting of Pd(PPh₃)₄ and Pd(PPh₃)₂Cl₂, and one or more copper catalysts selected from the group consisting of Cut, CuBr, and CuCl, wherein the palladium catalyst is in an amount of from 0.1 mol % to 0.5 mol % and the copper catalyst is in an amount of from 1 mol % to 5 mol %, to generate Compound (B); 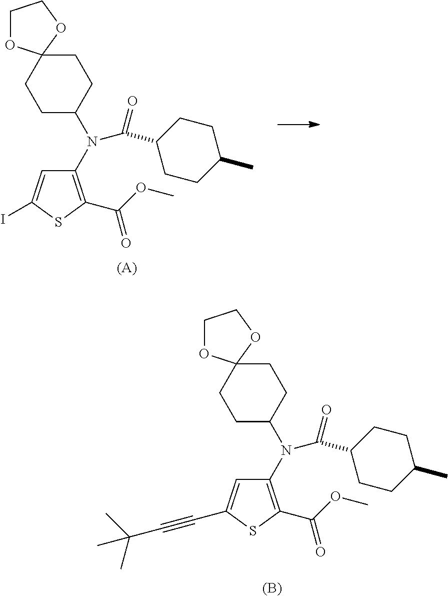
e) treating Compound (B) with an acid to generate Compound (C): 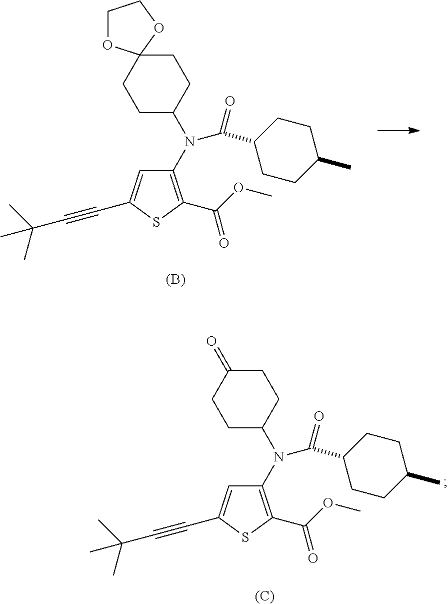
f) reducing the cyclohexanone of Compound (C) to cyclohexanol to generate Compound (D) by the use of LiAlH(O^{t}Bu)₃ in an amount of 1.0 to 1.5 equivalents based on molar amount of Compound (C) at a temperature in a range of −70° C. to −35° C., wherein ^{t}Bu is tert-butyl: 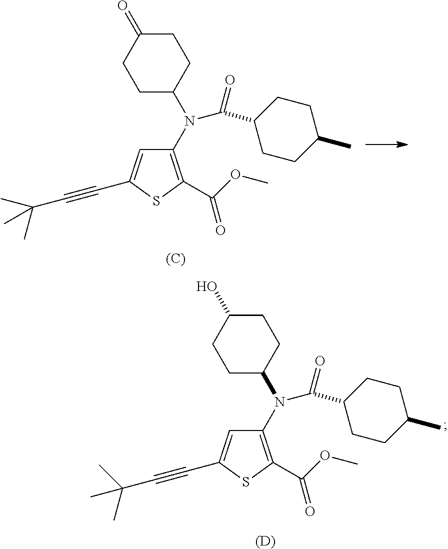
and
g) reacting Compound (D) with a base to generate Compound (1).

44-51. (canceled)

52. The method of claim 43 wherein the palladium catalyst in step d) is present in an amount of 0.2 mol %.

53. The method of claim 52 wherein the copper catalyst is present in an amount from 2.5 mol % to 5 mol %.

54. The method of claim 53 wherein 3-dimethylbut-1-yne in step d) is in an amount of 1 to 1.5 equivalent to Compound (A).

55-66. (canceled)

67. The method of claim 43, wherein the palladium catalyst is Pd(PPh₃)₄.

68. The method of claim 43, wherein the palladium catalyst is Pd(PPh₃)₂Cl₂.

69. The method of claim 68, wherein the copper catalyst is CuI.

70. The method of claim 43, further comprising crystallization of Compound (A) from toluene and heptane prior to step d).

71. The method of claim 43, further comprising crystallization of Compound (C) from a mixture of acetone, 2-butanone, and water prior to step f).

72-76. (canceled)

77. A method of preparing Compound (B): 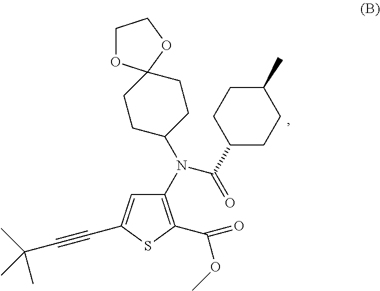
comprising reacting Compound (A) with 3,3-dimethylbut-1-yne in the presence of one or more palladium catalysts selected from the group consisting of Pd(PPh₃)₄ and Pd(PPh₃)₂Cl₂, and one or more copper catalysts selected from the group consisting of CuI, CuBr, and CuCl, to generate Compound (B):

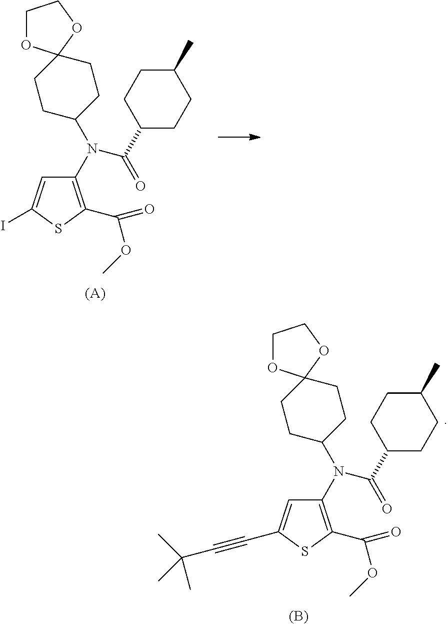

78-93. (canceled)
